SEQUENCE LISTING (4 sequences)

SEQ ID NO: 1 - protein, 766 aa, Homo sapiens [sequence omitted]

SEQ ID NO: 2 - protein, 766 aa, Homo sapiens
  MOD_RES (600)..(600): Any amino acid other than Val
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATXKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 3 - protein, 766 aa, Homo sapiens
  MOD_RES (601)..(601): Any amino acid other than Lys
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVXSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 4 - protein, 766 aa, Homo sapiens
  MOD_RES (597)..(597): Any amino acid other than Leu
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGXATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

(19) United States
(12) Patent Application Publication
(10) Pub. No.: US 2022/0378821 A1
(43) Pub. Date: Dec. 1, 2022

(54) METHODS OF TREATING BRAF-MUTATED CANCER CELLS

(71) Applicant: EFFECTOR THERAPEUTICS, INC., San Diego, CA (US)

(72) Inventors: Kevin R. WEBSTER, San Diego, CA (US); Gary CHIANG, San Diego, CA (US)

(21) Appl. No.: 17/624,504

(22) PCT Filed: Jun. 30, 2020
(86) PCT No.: PCT/US2020/040366

§ 371 Date: Jan. 3, 2022

Related U.S. Application Data
(60) Provisional application No. 62/869,894, filed on Jul. 2, 2019.

(51) Int. Cl. A61K 35/13; C12N 15/10; A61K 31/519

(52) U.S. Cl. CPC A61K 35/13; C12N 15/102; A61K 31/519

(57) ABSTRACT

The present disclosure provides methods for the treatment of a subject having BRAF-mutated cancer cells comprising administering an effective amount of an eIF4E inhibitor, which may be optionally used in combination with other therapies, such as RAF inhibitors. Furthermore, BRAF mutational status can be used to select for patients that would clinically benefit from eIF4E inhibition, such as patient with BRAF-mutated cancer cells that are resistant to RAF kinase inhibitors.

### CROSS-REFERENCES TO RELATED APPLICATIONS

This application is a U.S. national phase application of PCT International Application No. PCT/US2020/040366, filed on Jun. 30, 2020, which is an international application of and claims the benefit of priority to U.S. Provisional Patent Application No. 62/869,894, filed on Jul. 2, 2019, the entire contents of which are herein incorporated by reference.

### SEQUENCE LISTING

The instant application contains a Sequence Listing which has been submitted electronically in ASCII format and is hereby incorporated by reference in its entirety. Said ASCII copy, created on Jul. 24, 2020, is named 050040_520N01US_SL.txt and is 26,885 bytes in size.

### BACKGROUND

Mitogen-activated protein kinase (MAPK) is a key signaling pathway in a number of cancers. This pathway regulates important cell functions such as cellular growth, differentiation, proliferation, senescence, and apoptosis. BRAF, a serine threonine kinase and member of the RAF family of kinases, is a component of the MAPK pathway. It is estimated that 8% of all cancers have mutations in BRAF, and BRAF alterations have been described in numerous cancers, including melanoma (67%), colorectal (2%), thyroid (15%), non-small cell lung cancer (3%), serous ovarian cancer (30%), and hairy cell leukemia (100%). Activating mutations in BRAF lead to constitutive activation of BRAF and hence RAF-MEK-ERK signaling cascade, promoting cell proliferation and survival while inhibiting apoptosis, and thus driving cancer growth.

BRAF targeted therapy, such as vemurafenib and dabrafenib, are available for treating BRAF activated tumors. However, nearly 20% of patients do not respond to BRAF targeted therapy due to intrinsic resistance, and most responders to BRAF targeted therapy eventually acquire resistance.

There is a need in the art for alternative, effective methods for methods of treating cancers comprising BRAF-mutated cancer cells. The present disclosure meets such needs, and further provides other related advantages.

### BRIEF DESCRIPTION OF THE SEVERAL VIEWS OF THE DRAWINGS

This patent or application file contains at least one drawing executed in color. Copies of this patent or patent application publication with color drawing(s) will be provided by the Office upon request and payment of the necessary fee.

FIGS. 1A-B show differential sensitivity to eIF4E inhibition in a panel of cancer cell lines. FIG. 1A is a waterfall plot of cell proliferation IC₅₀ values of each cell line relative to the median IC₅₀ value (166 nM). Cell line identities are listed on the Y-axis and tumor type is denoted by color code. FIG. 1B is a table listing breakdown of cell line sensitivities grouped by tumor type and mutation status.

FIG. 2 shows that BRAF mutant cell lines show increased apoptosis in response to eIF4E inhibition. Waterfall plot of maximal activation of apoptosis (Apoptosis Eₘₐₓ) for each cell line as measured by fold change in activated caspase-3 relative to vehicle. The red vertical line denotes the 5-fold cut-off threshold for scoring caspase-3 activation as significant.

FIGS. 3A-B show in vivo efficacy of Compound Y in COLO 205 xenografts. COLO 205 xenograft-bearing animals were treated with vehicle or the indicated doses of Compound Y daily for the duration of the study. FIG. 3A shows tumor volumes over the duration of the study. FIG. 3B shows body weight measurements.

FIGS. 4A-B show in vivo efficacy of Compound Y in RKO xenografts. RKO xenograft-bearing animals were treated with vehicle or the indicated dose of Compound Y daily for the duration of the study. FIG. 4A shows tumor volumes over the duration of the study. FIG. 4B shows body weight measurements.

### DETAILED DESCRIPTION

The present disclosure provides methods for the treatment of BRAF-mutated cancer cells comprising the use of an eIF4E inhibitor. For example, activating mutations of BRAF deregulate the kinase activity of BRAF, resulting in constitutive activation and enhanced cell proliferation and survival and the development of cancer. Targeted BRAF inhibitors, such as vemurafenib and dabrafenib, are capable of inhibiting BRAF possessing an activating mutation at V600. However, approximately 20% of patients, who possess the V600 mutation or do not harbor the mutation, are intrinsically resistant to kinase inhibitors like vemurafenib and dabrafenib. Moreover, the durability of response to BRAF inhibitors is limited, with evidence of disease progression appearing within 6 to 8 months of starting therapy due to development of resistance (e.g., further sequence mutations in BRAF or amplification of the BRAF gene). To minimize or avoid the development of resistance by BRAF-mutated cancer cells, the present disclosure provides eIF4E inhibitors for use in treating a subject having BRAF-mutated cancer cells. Furthermore, BRAF mutational status can be used to select for patients that would clinically benefit from eIF4E inhibition, such as patient with BRAF-mutated cancer cells that are resistant to RAF kinase inhibitors.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)— or —C(═O)— group. Both notations are used interchangeably within the specification.

“Cyano” refers to the —C≡N substituent.

“Cyanoalkylene” refers to the -(alkylene)C≡N substituent.

“Acetyl” refers to the —C(O)CH₃ substituent.

“Hydroxy” or “hydroxyl” refers to the —OH substituent.

“Hydroxyalkylene” refers to the -(alkylene)OH substituent.

“Oxo” refers to a ═O substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Lower alkyl” has the same meaning as alkyl defined above but having from one to four carbon atoms (C₁-C₄ alkyl).

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“Alkynylene” refers to divalent alkyne. Examples of alkynylene include without limitation, ethynylene, propynylene. “Substituted alkynylene” refers to divalent substituted alkyne.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Acyl” refers to a radical of the formula —C(O)Rₐ where Rₐ is an alkyl having the indicated number of carbon atoms.

“Alkylaminyl” refers to a radical of the formula —NHRₐ or —NRₐRₐ where each Rₐ is, independently, an alkyl radical having the indicated number of carbon atoms as defined above.

“Cycloalkylaminyl” refers to a radical of the formula —NHRₐ where Rₐ is a cycloalkyl radical as defined herein.

“Alkylcarbonylaminyl” refers to a radical of the formula —NHC(O)Rₐ, where Rₐ is an alkyl radical having the indicated number of carbon atoms as defined herein.

“Cycloalkylcarbonylaminyl” refers to a radical of the formula —NHC(O)Rₐ, where Rₐ is a cycloalkyl radical as defined herein.

“Alkylaminocarbonyl” refers to a radical of the formula —C(O)NHRₐ or —C(O)NRₐRₐ, where each Rₐ is independently, an alkyl radical having the indicated number of carbon atoms as defined herein.

“Cyclolkylaminocarbonyl” refers to a radical of the formula —C(O)NHRₐ, where Rₐ is a cycloalkyl radical as defined herein.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of the compounds of the present disclosure, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group.

“Arylene” denotes divalent aryl, and “substituted arylene” refers to divalent substituted aryl.

“Aralkyl” or “araalkylene” may be used interchangeably and refer to a radical of the formula —R_{b}—Rₑ where R_{b} is an alkylene chain as defined herein and Rₑ is one or more aryl radicals as defined herein, for example, benzyl, diphenylmethyl and the like.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Cycloalkylalkylene” or “cycloalkylalkyl” may be used interchangeably and refer to a radical of the formula —R_{b}Rₑ where R_{b} is an alkylene chain as defined herein and Rₑ is a cycloalkyl radical as defined herein. In certain embodiments, R_{b} is further substituted with a cycloalkyl group, such that the cycloalkylalkylene comprises two cycloalkyl moieties. Cyclopropylalkylene and cyclobutylalkylene are exemplary cycloalkylalkylene groups, comprising at least one cyclopropyl or at least one cyclobutyl group, respectively.

“Fused” refers to any ring structure described herein which is fused to an existing ring structure in the compounds of the present disclosure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl,” “heterocycle,” or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heterocyclylalkyl” or “heterocyclylalkylene” refers to a radical of the formula —R_{b}R_{f} where R_{b} is an alkylene chain as defined herein and R_{f} is a heterocyclyl radical as defined above, and if the heterocyclyl is a nitrogen-containing heterocyclyl, the heterocyclyl may be attached to the alkyl radical at the nitrogen atom.

“Heteroaryl” or “heteroarylene” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of the compounds of the present disclosure, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, 2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

“Heteroarylalkyl” or “heteroarylalkylene” refers to a radical of the formula —R_{b}R_{g} where R_{b} is an alkylene chain as defined above and R_{g} is a heteroaryl radical as defined above.

“Thioalkyl” refers to a radical of the formula —SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms.

“Heterocyclylaminyl” refers to a radical of the formula —NHR_{f} where R_{f} is a heterocyclyl radical as defined above.

“Thione” refers to a ═S group attached to a carbon atom of a saturated or unsaturated (C₃-C₈)cyclic or a (C₁-C₈)acyclic moiety.

“Sulfoxide” refers to a —S(O)— group in which the sulfur atom is covalently attached to two carbon atoms.

“Sulfone” refers to a —S(O)₂— group in which a hexavalent sulfur is attached to each of the two oxygen atoms through double bonds and is further attached to two carbon atoms through single covalent bonds.

The term “oxime” refers to a —C(Rₐ)═N—ORₐ radical where Rₐ is hydrogen, lower alkyl, an alkylene or arylene group as defined above.

The compounds provided in the present disclosure can exist in various isomeric forms, as well as in one or more tautomeric forms, including both single tautomers and mixtures of tautomers. The term “isomer” is intended to encompass all isomeric forms of a compound of the present disclosure, including tautomeric forms of the compound.

Some compounds described herein can have asymmetric centers and therefore exist in different enantiomeric and diastereomeric forms. A compound provided in the present disclosure can be in the form of an optical isomer or a diastereomer. Accordingly, the invention encompasses compounds provided in the present disclosure and their uses as described herein in the form of their optical isomers, diastereoisomers and mixtures thereof, including a racemic mixture. Optical isomers of the compounds provided in the present disclosure can be obtained by known techniques such as asymmetric synthesis, chiral chromatography, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In this description, a “pharmaceutically acceptable salt” is a pharmaceutically acceptable, organic or inorganic acid or base salt of a compound of the present disclosure. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties from the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiqutination, esterification, and amidation.

The term “derivative” also refers to all solvates, for example, hydrates or adducts (e.g., adducts with alcohols), active metabolites, and salts of a parent compound. The type of salt depends on the nature of the moieties within the compound. For example, acidic groups, such as carboxylic acid groups, can form alkali metal salts or alkaline earth metal salts (e.g., sodium salts, potassium salts, magnesium salts, calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts with, for example, inorganic acids such as hydrochloric acid, sulfuric acid or phosphoric acid, or with organic carboxylic acids or sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

The term “prodrug” refers to a precursor of a drug, a compound which upon administration to a patient, must undergo chemical conversion by metabolic processes before becoming an active pharmacological agent. Exemplary prodrugs of compounds in accordance with, e.g., eIF4A inhibitor of Formula I, are esters, acetamides, and amides.

As used herein, the term “RAF kinase” (Rapidly Accelerated Fibrosarcoma kinase) or “RAF” refers to a family of serine/threonine-specific kinases, including A-RAF, B-RAF, and C-RAF (also known as RAF1). RAF kinases function in the Ras-Raf-MEK-ERK mitogen activated protein kinase (MAPK) signaling pathway, which plays a key role in regulating many cellular functions including cell proliferation, differentiation, and transformation. All RAF proteins share MEK1/2 kinases as substrates. In the prototypical Ras-Raf-MEK-ERK pathway, activated receptor tyrosine kinases (RTKs) recruit the guanine nucleotide exchange factor SOS, which activates RAS proteins by exchanging GDP for GTP. Activated GTP-loaded RAS binds to RAF, initiating RAF activation. Active RAF phosphorylates and activates MEK1/2, which in turn phosphorylates and activates ERK1/2. While the phosphorylation cascade comprising RAF, MEK, and ERK is linear, ERK features more than 150 substrates both in the cytosol and nucleus, including ELF1, FOS, JUN, AP1, and MYC. The ERK pathway regulates many cellular functions, such as cell proliferation, differentiation, migration, or apoptosis. RAF may refer to A-RAF or variants thereof, B-RAF or variants thereof, C-RAF or variants thereof, or any combination thereof. In certain embodiments, RAF refers to a human RAF.

As used herein, the term “BRAF” or “B-RAF” refers to a member of the RAF kinase family. BRAF is composed of three conserved domains characteristic of the RAF kinase family: conserved region 1 (CR1), conserved region 2 (CR2), and conserved region 3 (CR3). CR1 is a RAS-GTP binding self-regulatory domain that auto-inhibits BRAF's kinase domain. Amino acids 155-277 make up the RAS-binding domain, which binds to RAS-GTP and halts kinase inhibition. Amino acids 234-280 comprise a phorbol ester/DAG-binding zinc finger motif that participates in BRAF membrane docking after RAS-binding. CR2 is a serine-rich hinge region that provides a flexible linker connecting CR1 and CR3. CR3 (amino acids 457-717) comprises BRAF's catalytic kinase domain. The N-lobe of CR3 (amino acids 457-530) is primarily involved in ATP binding with the C-lobe (amino acids 535-717) binds kinase substrate proteins. The kinase active site lies in the cleft between the N-lobe and C-lobe. BRAF may refer to wildtype BRAF or variants thereof, including mutated BRAF (e.g., activating mutations, inactivating mutations, gene amplifications). In certain embodiments, a mutated BRAF refers to a mutated BRAF that is resistant to a BRAF inhibitor. In certain embodiments, BRAF refers to human BRAF. An exemplary wild type human BRAF protein is set forth in Uniprot Ref. P15056-1 (SEQ ID NO:1). As used herein, mutant BRAF polypeptides that refer to amino acid positions for substitutions refer to the amino acid position of the wildtype human BRAF polypeptide sequence (SEQ ID NO:1).

As used herein, the term “MEK”, also known as MAP2K, MAPKK, or mitogen-activated protein kinase kinase, refers to a dual threonine and tyrosine recognition kinase that phosphorylates and ERK. MEK is phosphorylated and activated by RAF kinases. MEK may refer to MEK1, MEK2, or both. In certain embodiments, MEK refers to a human MEK.

As used herein, the term “KRAS” or “K-RAS” refers to a member of the RAS family of GTPases that is involved in signal transduction for cell growth, differentiation, and survival. There are two protein isoforms of KRAS due to the use of alternative exon 4: KRAS4A and KRAS4B. KRAS acts as a membrane localized molecular switch, where following EGF binding to its receptor and activation of tyrosine kinases, KRAS becomes activated by binding to GTP, transducing the activation signal to the nucleus by the Raf-MEK-ERK signaling cascade. KRAS may refer to a wildtype KRAS, isoforms, or variants thereof, including mutated KRAS. In certain embodiments, KRAS refers to a human KRAS.

As used herein, the term “MNK,” also known as “mitogen-activated protein kinase (MAPK)-interacting serine/threonine kinase” or “MKNK” refers to a kinase that is phosphorylated by the p42 MAP kinases ERK1 and ERK2 and the p38-MAP kinases, triggered in response to growth factors, phorbol esters, and oncogenes such as Ras and Mos, and by stress signaling molecules and cytokines. MNK also refers to a kinase that is phosphorylated by additional MAP kinase(s) affected by interleukin-1 receptor-associated kinase 2 (IRAK2) and IRAK4, which are protein kinases involved in signaling innate immune responses through toll-like receptors (e.g., TLR7) (see, e.g., Wan et al., J. Biol. Chem. 284: 10367, 2009). Phosphorylation of MNK proteins stimulates their kinase activity toward eukaryotic initiation factor 4E (eIF4E), which in turn regulates cap-dependent protein translation initiation, as well as regulate engagement of other effector elements, including hnRNPA1 and PSF (PTB (polypyrimidine tract binding protein) associated splicing factor). For example, proteins that bind the regulatory AU-rich elements (AREs) of the 3′-UTR of certain mRNAs (e.g., cytokines) are phosphorylated by MNK. Thus, MNK phosphorylation of proteins can alter the ability of these proteins to bind the 5′- or 3′-UTRs of eukaryotic mRNAs. In particular, reduced MNK mediated phosphorylation of hnRNPA1 decreases its binding to cytokine-ARE (see, e.g., Buxade et al., Immunity 23:177, 2005; Joshi and Platanias, Biomol. Concepts 3:127, 2012). MNK is encoded by two different genes, MNK1 and MNK2, which are both subject to alternative splicing. MNK1a and MNK2a represent full length transcripts, while MNK 1b and MNK2b are splice variants that lack a MAPK binding domain. Therefore, MNK may refer to MNK1 or variants thereof (such as MNK1a or MNK1b), MNK2 or variants thereof (such as MNK2a or MNK2b), or combinations thereof. In particular embodiments, MNK refers to human MNK.

As used herein, “eIF4A,” also known as “eukaryotic initiation factor-4A,” refers to a member of the “DEAD box” family of ATP-dependent helicases that are characterized by seven highly conserved amino acid motifs implicated in RNA remodeling. eIF4A acts as an RNA dependent ATPase and ATP-dependent RNA helicase to facilitate mRNA binding to the ribosome as part of the eIF4F (eukaryotic initiation factor 4F) complex that recognizes and initiates translation of most cellular mRNAs to synthesize specific proteins. A functional eIF4F complex consisting of eIF4A, eIF4E and eIF4G is involved in translation of mRNAs that contain highly structured 5′-UTRs or an IRES element. In particular, eIF4F recognizes the cap structure at the 5′-end of mRNA through eIF4E, unwinds the secondary structure of the 5′-UTR region through the helicase activity of eIF4A, and binds the 43S complex through interactions between eIF4G and eIF3. See, e.g., Marintchev et al., Cell, 136: 447-460, 2009, and Parsyan et al., Nat. Rev. Mol. Cell Biol. 12:235-245, 2012. eIF4A selectively regulates the translation of a subset of mRNAs. This selectivity is a result of structural elements and sequence recognition motifs found within the 5′-UTR of the mRNA. There are three eIF4A family members: eIF4AI, eIF4AII, and eIF4AIII In particular embodiments, eIF4A refers to human eIF4A.

As used herein, the term “eIF4E,” also referred to as “eukaryotic translation initiation factor-4E,” refers to a translation initiation factor that, when part of an eIF4F pre-initiation complex also comprising eIF4A RNA helicase and eIF4G scaffold protein, binds to the 7-methyl-guanosine (m7GpppX) 5′-cap structure on eukaryotic mRNAs and directs ribosomes to the cap structure. The availability of eIF4E as part of the eIF4F complex is a limiting factor in controlling the rate of translation. Interactions of eIF4E and the m⁷G cap and eIF4G are tightly regulated by key mitogenic signals, such as the PI3K/mTOR and Ras/MAPK signal transduction pathways. There are four different isoforms of eIF4E: isoform 1 is the canonical sequence; isoform 2 contains an alternate in-frame exon in the 3′-coding region compared to isoform 1; isoform 3 uses an alternate 5′-terminal exon, which results in a different 5′-UTR and use of an alternate translation start codon compared to isoform 1; and isoform 4 differs in its 5′-UTR and contains an alternate exon in its 5′-coding region compared to isoform 1. In certain embodiments, eIF4E refers to the canonical eIF4E isoform 1. In particular embodiments, eIF4E refers to human eIF4E.

As used herein, the term “mTOR,” also known as “mammalian target of rapamycin,” also known as “FK506-binding protein 12-rapamycin-associate protein 1” (FRAP1), refers to a serine/threonine kinase that is a member of the phosphatidylinositol 3-kinase-related kinase family that is encoded by the mTOR gene. mTOR functions as part of two structural and functionally distinct signaling complexes—mTOR complex 1 (mTORC1) and mTOR complex 2 (mTORC2). mTORC1 is composed of mTOR, Raptor, GPL, and DEPTOR, and is inhibited by rapamycin. Activated mTORC1 up-regulates protein synthesis by phosphorylating key regulators of mRNA translation and ribosome synthesis, including phosphorylation of EIF4EBP1 and release of its inhibition toward the elongation initiation factor 4E (eIF4E). mTORC2 is composed of mTOR, Rictor, GβL, Sin1, PRR5/Protor-1, and DEPTOR. Reference to mTOR may refer to mTOR as a component of mTORC1, as a component of mTORC2, or both. In particular embodiments, mTOR refers to human mTOR.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methylsulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double stranded. If single stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

As used herein, the term “agent” refers to any molecule, either naturally occurring or synthetic, e.g., peptide, protein, oligopeptide (e.g., from about 5 to about 25 amino acids in length, preferably from about 10 to 20 or 12 to 18 amino acids in length, preferably 12, 15, or 18 amino acids in length), small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound.

The term “inhibit” or “inhibitor” refers to an alteration, interference, reduction, down regulation, blocking, suppression, abrogation or degradation, directly or indirectly, in the expression, amount or activity of a target gene, target protein, or signaling pathway relative to (1) a control, endogenous or reference target or pathway, or (2) the absence of a target or pathway, wherein the alteration, interference, reduction, down regulation, blocking, suppression, abrogation or degradation is statistically, biologically, or clinically significant. The term “inhibit” or “inhibitor” includes gene “knock out” and gene “knock down” methods, such as by chromosomal editing.

“Treatment,” “treating” or “ameliorating” refers to medical management of a disease, disorder, or condition of a subject (i.e., patient), which may be therapeutic, prophylactic/preventative, or a combination treatment thereof. A treatment may improve or decrease the severity at least one symptom of a disease, delay worsening or progression of a disease, or delay or prevent onset of additional associated diseases. “Reducing the risk of developing a disease” refers to preventing or delaying onset of a disease or reoccurrence of one or more symptoms of the disease (e.g., cancer).

As used herein, the term “immune suppression component” or “immunosuppression component” refers to one or more cells, proteins, molecules, compounds or complexes providing inhibitory signals to assist in controlling or suppressing an immune response. For example, immunosuppression components include those molecules that partially or totally block immune stimulation; decrease, prevent or delay immune activation; or increase, activate, or up regulate immune suppression. Exemplary immunosuppression component targets include immune checkpoint ligands (such as PD-L1, PD-L2, CD80, CD86, B7-H3, B7-H4, HVEM, adenosine, GALS, VISTA, CEACAM-1, PVRL2), immune checkpoint receptors (such as PD-1, CTLA-4, BTLA, KIR, LAG3, TIN/13, A2aR, CD244/2B4, CD160, TIGIT, LAIR-1, PVRIG/CD112R), metabolic enzymes (such as arginase, indoleamine 2,3-dioxygenase (IDO)), immunosuppressive cytokines (such as IL-10, IL-4, IL-1RA, IL-35), T_{reg} cells, or any combination thereof. In certain embodiments, an immunosuppression component is an immune checkpoint molecule, which may initiate an immune suppression signal through a ligand-receptor interaction, such as by modulating (e.g., inhibiting) an antigen-specific T cell response. For example, a T cell may express on its surface an immune checkpoint receptor (e.g., PD-1, LAG3) and an antigen presenting cell may express on its surface an immune checkpoint receptor ligand (e.g., PD-L1, MHC/HLA molecule). In further embodiments, an immunosuppression component is a metabolic enzyme that inhibits immune responses through the local depletion of amino acids essential for lymphocyte, particularly T cell, survival and function. In still further embodiments, an immunosuppression component may be a signaling molecule, such as an immunosuppressive cytokine (e.g., IL-10, IL-4, IL-1RA, IL-35). In still further embodiments, an immunosuppression component comprises a CD4⁺ T_{reg} cell that is capable of inhibiting an immune response, as well as producing or releasing immunosuppressive cytokines (e.g., IL-10, IL-4, IL-13, IL-1RA).

A “patient” or “subject” includes an animal, such as a human, cow, horse, sheep, lamb, pig, chicken, turkey, quail, cat, dog, mouse, rat, rabbit or guinea pig. The animal can be a mammal, such as a non-primate and a primate (e.g., monkey and human). In one embodiment, a patient is a human, such as a human infant, child, adolescent or adult.

“Effective amount” or “therapeutically effective amount” refers to that amount of a composition described herein which, when administered to a mammal (e.g., human), is sufficient to aid in treating a disease. The amount of a composition that constitutes a “therapeutically effective amount” will vary depending on the cell preparations, the condition and its severity, the manner of administration, and the age of the mammal to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

As used herein, “hyperproliferative disorder” or “hyperproliferative disease” refers to excessive growth or proliferation as compared to a normal cell or an undiseased cell. Exemplary hyperproliferative disorders include dysplasia, neoplasia, non-contact inhibited or oncogenically transformed cells, tumors, cancers, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, or the like). In certain embodiments, a cancer being treated by the compositions and methods of this disclosure includes carcinoma (epithelial), sarcoma (connective tissue), lymphoma or leukemia (hematopoietic cells), germ cell tumor (pluripotent cells), blastoma (immature “precursor” cells or embryonic tissue), or any combination thereof. These various forms of hyperproliferative disease are known in the art and have established criteria for diagnosis and classification (e.g., Hanahan and Weinberg, Cell 144:646, 2011; Hanahan and Weinberg Cell 100:57, 2000; Cavallo et al., Canc. Immunol. Immunother, 60:319, 2011; Kyrigideis et al., J. Carcinog. 9:3, 2010).

### A. BRAF-Mutated Cancer Cells

In one aspect, the present disclosure provides a method of treating cancer, the method comprising administering to a subject having BRAF-mutated cancer cells an effective amount of an eIF4E inhibitor.

As used herein, a “BRAF-mutation” or “aberrant BRAF” or “BRAF-mutated cancer cell” or “aberrant BRAF associated cancer” refer to alterations to a wild-type or parent BRAF gene located on a genome or extrachromosomal element, or to the encoded BRAF polypeptide, which may include alterations to the parent polynucleotide sequence encoding BRAF, alterations to the parent polypeptide sequence of BRAF, alterations to the parent polynucleotide sequence involved in BRAF expression, multiplication or amplification in the number of BRAF genes, multiplication or amplification in the number of BRAF genes having one or more polynucleotide sequence mutations, or the like. A “BRAF-mutation” or “aberrant BRAF” may or may not result in altered function of the encoded protein or in an observable phenotype. Examples of polynucleotide sequence mutations include missense mutations, nonsense mutations, splice site mutations, silent mutations, insertion mutations, deletion mutations, substitution mutations, promoter mutations, partial or whole gene duplication (or amplification) mutations, frameshift mutations, repeat expansion mutations, inversion mutations, and translocation mutations. A sequence mutation may affect a single nucleotide (point mutation), a few nucleotides, tens of nucleotides, hundreds of nucleotides, the entire gene sequence, or a chromosomal segment. A mutation may occur in coding DNA or non-coding DNA. A BRAF-mutated cancer cell may comprise one or more BRAF mutations (e.g. a sequence mutation, an amplification mutation, or a combination thereof). A plurality of BRAF-mutated cancer cells in a subject may be composed of population of cells that each comprise the same BRAF mutation(s) or a population of cells having heterogeneous BRAF mutations.

In certain embodiments, the BRAF-mutated cancer cell comprises an amino acid substitution at position MI17, 1326, K439, T440, V459, R462, 1463, G464, G466, F468, G469, Y472, K475, N581, E586, D587, D594, F595, G596, L597, T599, V600, K601, R682, A728, or any combination thereof in the BRAF polypeptide. In certain embodiments, the position of the amino acid substitution in the BRAF polypeptide refers to a position in SEQ ID NO:1.

In certain embodiments, the M117 substitution is a M117R substitution.

In certain embodiments, the 1326 substitution is a I326T substitution.

In certain embodiments, the K439 substitution is a K439Q or K439T substitution.

In certain embodiments, the T440 substitution is a T440P substitution.

In certain embodiments, the V459 substitution is a V459L substitution.

In certain embodiments, the R462 substitution is a R462I substitution.

In certain embodiments, the 1463 substation is an I463S substitution.

In certain embodiments, the G464 substitution is a G464E, G464V, or G464R substitution.

In certain embodiments, the G466 substitution is a G466A, G466E, G466R, or G466V substitution.

In certain embodiments, the F468 substitution is a F468C substitution.

In certain embodiments, the G469 substitution is a G469A, G469E, G469R, G469S, or G469V substitution.

In certain embodiments, the K475 substitution is a K475E substitution.

In certain embodiments, the N581 substitution is a N581S substitution.

In certain embodiments, the E586 substitution is a E586K substitution.

In certain embodiments, the D587 substitution is a D587A substitution.

In certain embodiments, the D594 substitution is a D594E, D594G, D594H, D594K, D594N, or D594V substitution.

In certain embodiments, the F595 substitution is a F595L substitution.

In certain embodiments, the G596 substitution is a G596R substitution.

In certain embodiments, the L597 substitution is a L597Q, L597R, L597S, or L597V substitution. In further embodiments, the mutated BRAF comprises a L597Q, L597R, L597S, or L597V substitution in SEQ ID NO:4.

In certain embodiments, the T599 substitution is a T599I substitution.

In certain embodiments, the V600 substitution is a V600E, V600D, V600A, V600G, V600K, V600L, V600M, or V600R substitution. In further embodiments the mutated BRAF comprises a V600E, V600D, V600A, V600G, V600K, V600L, V600M, or V600R substitution in SEQ ID NO:2.

In certain embodiments, the K601 substitution is a K601E or a K601N substitution. In further embodiments, the mutated BRAF comprises a K601N substitution in SEQ ID NO:3.

In certain embodiments, the R682 substitution is a R682Q substitution.

In certain embodiments, the A728 substitution is an A728V substitution.

In certain embodiments, the BRAF-mutated cancer cells comprise a mutation that activates BRAF. An activating mutation increases expression of a protein product, results in inappropriate expression of the protein product, or increased or inappropriate activity of the protein product. An activating mutation may result from a constitutively acting protein product, gain in copy number (e.g., amplification mutation), inappropriate expression of the gene due to mutation of or switching of expression control elements (e.g., promoter). Upregulation of BRAF signaling, resulting from an activating mutation in BRAF or aberrant signaling through RAS, promotes oncogenesis by activating the RAS-RAF-MEK-ERK signaling cascade resulting in increased cell proliferation and survival.

In certain embodiments, a BRAF activating mutation comprises an amino acid substitution at position F595, L597, V600, K601, or any combination thereof in the BRAF polypeptide.

In certain embodiments, the F595 substitution is a F595L substitution.

In certain embodiments, the L597 substitution is a L597Q, L597R, L597S, or L597V substitution.

The most prevalent mutation in BRAF is a missense substitution at codon 600, which occurs in 90% of all BRAF mutations. A V600 substitution results in a constitutively active BRAF. In certain embodiments, the V600 substitution is a V600E, V600D, V600A, V600G, V600K, V600L, V600M, or V600R substitution.

In certain embodiments, the K601 substitution is a K601E or a K601N substitution.

In certain embodiments, an activating BRAF mutation comprises gene amplification or duplication of the BRAF gene. In certain embodiments, amplification of a BRAF gene comprises 2, 3, 4, 5, 6, 7, 8, 9, 10, 11, 12, 13, 14, 15, 16, 17, 18, 19, 20 or more copies of the BRAF gene. In certain embodiments, the BRAF gene is amplified on a chromosome. In certain embodiments, a BRAF gene is amplified or duplicated on an extrachromosomal element. Amplified copies of BRAF may further comprise sequence mutations of BRAF, e.g., substitutions, activating mutations, inactivating mutations, etc.

In certain embodiments, the BRAF-mutated cancer cells comprise a mutation that inactivates BRAF. An inactivating BRAF mutation decreases expression or activity of a protein product. In certain embodiments, an inactivating BRAF mutation comprises an amino acid substitution at position G466, Y472, D594, G596, or any combination thereof in the BRAF polypeptide.

In certain embodiments, the G466 substitution is a G466A, G466E, G466R, or G466V substitution.

In certain embodiments, the D594 substitution is a D594E, D594G, D594H, D594K, D594N, or D594V substitution.

In certain embodiments, the G596 substitution is a G596R substitution.

In certain embodiments, the Y472 substitution is a Y472C substitution.

In certain embodiments, the BRAF-mutated cancer cell is resistant to a RAF inhibitor. In certain embodiments, the BRAF-mutated cancer cell comprises a mutated BRAF polypeptide having one or more amino acid substitutions occurring at one or more of the following positions A29, H72, 5113, 5124, P162, C194, L227, P231, C251, V291, Q329, V483, L485, T521, V528, D587, P655, 5657, 5683, P686, C696, L697, P722, F738, and C748 of a BRAF polypeptide that is a wild-type BRAF polypeptide (SEQ ID NO:1), a BRAF V600 polypeptide (SEQ ID NO:2), a BRAF K601 polypeptide (SEQ ID NO:3), or a BRAF L597 polypeptide (SEQ ID NO:4). In certain embodiments, the one or more amino acid substitutions of the BRAF polypeptide are selected from the group consisting of A29V, H72N, SI 131, S124F, P162H, C194*, L227F, P231T, C251F, V291F, Q329K, V483E, L485F, T521K, V528F, D587E, P655T, S657*, S683R, P686Q, P686T, C696*, L697I, P722T, F738L, and C748F, wherein * is any amino acid. In a specific embodiment, the mutated BRAF polypeptide comprises a substitution at one or more amino acid positions T521, V528, and P686. In a further embodiment, the mutant BRAF polypeptide comprises one or more amino acid substitutions T521K, V528F, and P686Q.

In certain embodiments, a RAF inhibitor is a selective BRAF inhibitor. In certain embodiments, the RAF inhibitor is an inhibitor that targets a mutant BRAF comprising a substitution at V600 (e.g., V600E). In certain embodiments, a BRAF inhibitor that targets BRAF comprising a V600 mutation is vemurafenib, dabrafenib, encorafenib, or RAF-265. A mutant BRAF polypeptide that is resistant to treatment with a RAF inhibitor exhibits greater BRAF activity (e.g., at least about 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 1000% or more) in the presence of the RAF inhibitor than a wild-type BRAF polypeptide or a BRAF V600E polypeptide in the presence of the RAF inhibitor.

Activity of a BRAF polypeptide can be determined by, for example, measuring proliferation or viability of cells following treatment with the RAF inhibitor, wherein proliferation or viability are positively correlated with RAF activity. For example, cell growth can be determined using well-based cell proliferation/viability assays such as MTS (344,5-dimethylthiazol-2-yl)-5-(3-carboxymethoxyphenyl)-2-(4-sulfophenyl)-2H-tetrazolium) assay, acolorimetric assay for measuring viable cells or CELLTITER-GLO™, wherein cell growth in the presence of a RAF inhibitor is compared to untreated cells cultured in the absence of the RAF inhibitor. Activity of a BRAF polypeptide can also be measured by, for example, determining the relative amount of phosphorylated MEK1/2 or ERK1/2 present in the cell following treatment with the RAF inhibitor. Activity of a wild-type or mutant BRAF polypeptide can also be determined using an in vitro phosphorylation assay, in which BRAF activity is determined by measuring the proportion of phosphorylated MEK1/2 or ERK1/2 in the assay following treatment with the BRAF inhibitor. A mutant BRAF polypeptide having greater activity than a wild-type BRAF polypeptide or a mutated BRAF V600E polypeptide following treatment with a RAF inhibitor is identified as containing a mutation that confers resistance to a RAF inhibitor.

In certain embodiments, a BRAF-mutated cancer cell does not have an activating KRAS mutation. In certain embodiments, the activating KRAS activating mutation not present in a BRAF-mutated cancer cell comprises an amino acid substitution at position G12, G13, Q61, or any combination thereof in the KRAS polypeptide. In further embodiments, the KRAS activating mutation not present in a BRAF-mutated cancer cell comprises an amino acid substitution of G12C, G12A, G12D, G12R, G12S, G12V, G13C, G13R, G13S, G13A, G13D, Q61K, Q61L, Q61R, Q61H, or any combination thereof.

B. eIF4E Inhibitors

The present disclosure provides methods for treating a subject having BRAF-mutated cancer cells with an effective amount of an eIF4E inhibitor. An “eIF4E inhibitor” is an agent or compound that directly interacts with eIF4E and may block, inactivate, reduce or minimize eIF4E activity (e.g., initiation of cap-dependent translation or translational effects), or reduce activity by promoting degradation of eIF4E, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated eIF4E.

In certain embodiments, an eIF4E inhibitor inhibits eIF4E activity by blocking eIF4E interaction with eIF4G, thus inhibiting formation of the eIF4F complex. Examples of eIF4E-eIF4G interaction inhibitors include thiazol hydrazones (Chen et al., Bioorganic Medicinal Chem. Lett. 14:5401-5405, 2004, which compounds are incorporated herein by reference in their entirety); compound EGI-1 (U.S. Pat. No. 8,257,931, which compound is incorporated herein by reference in its entirety); eIF4G1 peptide fragments (e.g., eIF4G₅₆₉₋₅₈₀) (U.S. Pat. No. 7,141,541, which peptides are incorporated herein by reference in their entirety); eIF4G1 peptides that have been modified to stabilize the c-helix (PCT Publication No. WO 2011/136744, which peptides are incorporated herein by reference in their entirety); and cross-linked eIF4G1 peptides (PCT Publication No. WO 2014/149001, which peptides are incorporated herein by reference in their entirety).

In some embodiments, an eIF4E inhibitor blocks binding of eIF4E to a mRNA cap. Examples of cap binding inhibitors are briciclib (Jasani et al., Cancer Res., 75(15 Suppl):Abstract No. 1649, 2015) and Ribivirin (Kentsis et al., Proc. Nat'l. Acad. Sci. U.S.A. 101:18105-10, 2004).

Further examples of eIF4E inhibitors that block binding to the mRNA cap include compounds disclosed in U.S. application Ser. No. 16/916,820 (claiming priority to U.S. Provisional Application No. 62/869,662), which compounds and synthetic methods disclosed therein are incorporated herein by reference in their entirety.

In some embodiments, an eIF4E inhibitor includes compounds according to Formula I

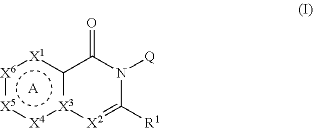

or stereoisomers, tautomers, or pharmaceutically acceptable salts thereof, wherein:

X¹ is CR², —C-L¹-Y or N;

X², X⁵ and X⁶ are independently CR² or N,
wherein X⁵ and X⁶ together with 3 or 4 carbon or nitrogen atoms combine to form a 5- or 6-membered cycloalkyl or heterocyclyl,
or when X² is CR², R¹ and R² together with the atoms they attached to form a 6-membered aryl or heteroaryl;

X³ is C, or X³ is C or N when X⁴ is a bond;

X⁴ is a bond, CR² or N,
wherein X⁴ and X⁵ together with 3 or 4 carbon or nitrogen atoms combine to form a 5- or 6-membered heteroaryl;

Q is H or —L¹-Y;

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂CC— or —CH₂(cyclopropyl)—;

Y is

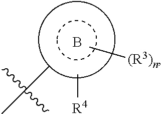

wherein

Ring B is a six-membered aryl, heteroaryl or heterocyclyl;

R¹ is H, OH, halo, CN, (C₁-C₈)alkyl, (C₁-C₈)haloalkyl, (C₃-C₆)cycloalkyl or NR⁵R⁵;

R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, NHR⁷,NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁴ is H, OH, halo, CN, (C₁-C₃)alkyl, (C₁-C₃)haloalkyl, (C₁-C₃)alkoxy, SR⁷ or Z, wherein Z is

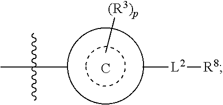

Ring C is cycloalkyl, heterocyclyl, aryl or heteroaryl;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;

R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
or wherein two R⁵ substituents together with a nitrogen atom form a 4-, 5-, 6- or 7-membered heterocyclyl;

R⁶ is independently H, OH, halo, CN, (C₁-C₃)alkyl, (C₁-C₃)haloalkyl, (C₁-C₃)alkoxy, NEM⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;

n is 0, 1, 2 or 3;

p is 0, 1, 2 or 3;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl,

wherein when X⁴ is a bond ring A forms a 5-membered heteroaryl wherein X¹, X⁵ and X⁶ can in addition to the above defined substituents be NR², and X¹ can in addition be —N-L¹-Y; and

wherein either Q is -L¹-Y, or X¹ is —C-L¹-Y or —N-L¹-Y.

In certain embodiments, the eIF4E inhibitor includes compounds according to Formula II

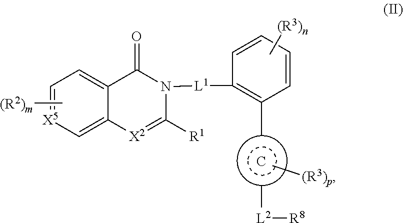

or stereoisomers, tautomers, or pharmaceutically acceptable salts thereof, wherein:

X² and X⁵ are independently CR² or N,
or when X² is CR², le and R² together with the atoms they attached to form a 6-membered aryl or heteroaryl;

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;

Ring C is cycloalkyl, heterocyclyl, aryl or heteroaryl;

R¹ is H, OH, halo, CN, (C₁-C₈)alkyl, (C₁-C₈)haloalkyl, (C₃-C₆)cycloalkyl or NR⁵R⁵;

R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,

or wherein two R⁵ substituents together with a nitrogen atom form a 4-, 5-, 6-, or 7-membered heterocyclyl;

R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;

m is 0, 1, 2 or 3;

n is 0, 1, 2 or 3;

p is 0, 1, 2 or 3;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

In certain embodiments, the eIF4E inhibitor includes compounds according to Formula III

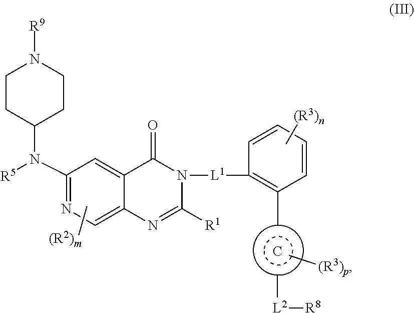

or stereoisomers, tautomers, or pharmaceutically acceptable salts thereof, wherein:

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;

Ring C is a heteroaryl;

R¹ is H, OH, halo, CN, (C₁-C₈)alkyl, (C₁-C₈)haloalkyl, (C₃-C₆)cycloalkyl or NR⁵R⁵;

R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, C≡H, NHR⁷,NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁵ is independently H, (C₁-C₃)alkyl, (C₁-C₃)haloalkyl, (C₃-C₅)cycloalkyl or heterocyclyl;

R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;

R⁹ is H, (C₁-C₈)alkyl, (C₁-C₈)haloalkyl, cycloalkyl or heterocyclyl;

m is 0, 1, or 2;

n is 0, 1, 2 or 3;

p is 0, 1, 2 or 3;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl.

In certain embodiments, the eIF4E inhibitor includes compounds according to Formula IV

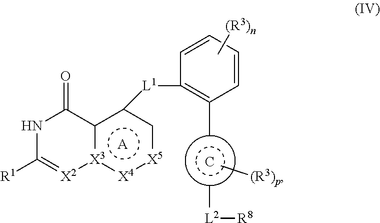

or stereoisomers, tautomers, or pharmaceutically acceptable salts thereof, wherein:

X² and X⁵ are independently CR² or N,
or when X² is CR², le and R² together with the atoms they attached to form a 6-membered aryl or heteroaryl;

X³ is C, or X³ is C or N when X⁴ is a bond;

X⁴ is a bond, CR² or N,
wherein X⁴ and X⁵ together with 3 or 4 carbon or nitrogen atoms combine to form a 5- or 6-membered heteroaryl;

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)—;

Ring C is cycloalkyl, heterocyclyl, aryl or heteroaryl;

R¹ is H, OH, halo, CN, (C₁-C₈)alkyl, (C₁-C₈)haloalkyl, (C₃-C₆)cycloalkyl or NR⁵R⁵;

R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, NHR⁷,NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl, or wherein two R⁵ substituents together with a nitrogen atom form a 4-, 5-, 6- or 7-membered heterocyclyl;

R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;

n is 0, 1, 2 or 3;

p is 0, 1, 2 or 3;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl,

wherein when X⁴ is a bond, ring A forms a 5-membered heteroaryl wherein X¹ and X⁵ can in addition to C be N.

In certain embodiments, the eIF4E inhibitor includes compounds according to Formula V

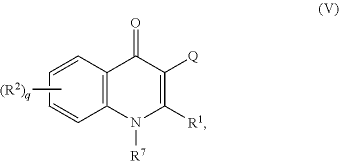

or stereoisomers, tautomers, or pharmaceutically acceptable salts thereof, wherein:

Q is -L¹-Y;

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;

Y is

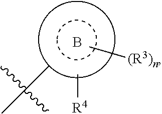

wherein

Ring B is a six-membered aryl, heteroaryl or heterocyclyl;

R¹ is H, OH, halo, CN, (C₁-C₈)alkyl, (C₁-C₈)haloalkyl, (C₃-C₆)cycloalkyl or NR⁵R⁵;

R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, C≡H, NHR⁷,NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁴ is H, OH, halo, CN, (C₁-C₃)alkyl, (C₁-C₃)haloalkyl, (C₁-C₃)alkoxy, SR⁷ or Z, wherein

Z is

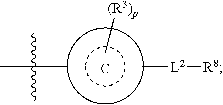

Ring C is cycloalkyl, heterocyclyl, aryl or heteroaryl;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;

R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,

or wherein two R⁵ substituents together with a nitrogen atom form a 4-, 5-, or 6-membered heterocyclyl;

R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, P(O)(OR⁵)OH, P(O)(O)N(H)R⁵, P(O)(C(R⁶)₃)C(R⁶)₃, B(OH)₂, heterocyclyl or heteroaryl;

n is 0, 1, 2 or 3;

p is 0, 1, 2 or 3;

q is 0, 1, 2, 3 or 4;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

In certain embodiments, the eIF4E inhibitor includes compounds according to Formula VI

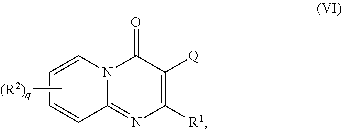

or stereoisomers, tautomers, or pharmaceutically acceptable salts thereof, wherein:

Q is -L¹-Y;

L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;

Y is

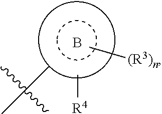

wherein

Ring B is a six-membered aryl, heteroaryl or heterocyclyl;

R¹ is H, OH, halo, CN, (C₁-C₈)alkyl, (C₁-C₈)haloalkyl, (C₃-C₆)cycloalkyl or NR⁵R⁵; R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;

R³ is independently OH, halo, CN, NO₂, (C₁-C₆)alkyl, (C₁-C₆)haloalkyl, (C₁-C₆)alkoxy, C≡H, NHR⁷,NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;

R⁴ is H, OH, halo, CN, (C₁-C₃)alkyl, (C₁-C₃)haloalkyl, (C₁-C₃)alkoxy, SR⁷ or Z, wherein Z is

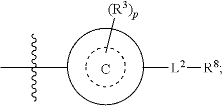

Ring C is cycloalkyl, heterocyclyl, aryl or heteroaryl;

L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;

R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,

or wherein two R⁵ substituents together with a nitrogen atom form a 4-, 5-, or 6-membered heterocyclyl;

R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;

R⁷ is independently H, (C₁-C₈)alkyl, (C₁-C₈)haloalkyl, cycloalkyl, heterocyclyl, aryl or heteroaryl;

R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;

n is 0, 1, 2 or 3;

p is 0, 1, 2 or 3;

q is 0, 1, 2, 3 or 4;

wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

In one embodiment X² of Formulae I, II, and IV is N.

In one embodiment X³ of Formulae I and IV is C.

In one embodiment X⁴ of Formulae I and IV is CR² or N.

In one embodiment X⁵ of Formulae I and IV is CR².

In one embodiment L¹ of Formulae I, II, III, IV, V and VI is —(CH₂)₂—O—, —CH₂CH═CH— or —CH₂CC—. In another embodiment L¹ is —(CH₂)₂—O—.

In one embodiment L² of Formulae I, II, III, IV, V and VI is a bond.

In one embodiment Ring B of Formulae I, V and VI is aryl.

In one embodiment Ring C of Formulae I, II, III, IV, V and VI is heteroaryl.

In one embodiment Ring C of Formulae I, II, III, IV, V and VI is

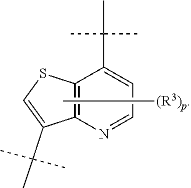

In one embodiment Ring C of Formula III is

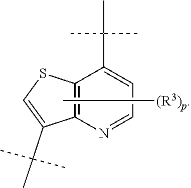

In one embodiment R¹ of Formulae I, II, III, IV, V and VI is H, (C₁-C₈)alkyl or (C₁-C₈)haloalkyl.

In one embodiment R¹ of Formula IV is NHR⁵ or N[(C₁-C₃)alkyl](R⁵).

In one embodiment R² of Formulae I, II, III, IV, V and VI is halo, CN, (C₁-C₈)alkyl, (C₁-C₈)haloalkyl or OR⁵. In another embodiment R² is halo, CN or (C₁-C₈)haloalkyl.

In one embodiment R³ of Formulae I, II, III, IV, V and VI is halo, CN, (C₁-C₃)alkyl or (C₁-C₃)haloalkyl.

In one embodiment R⁴ of Formulae I, V and VI is Z, wherein Z is

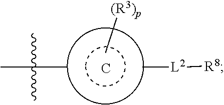

In one embodiment R⁵ of Formulae I, II, III, V and VI is H, (C₁-C₃)alkyl or (C₁-C₃)haloalkyl. In another embodiment R⁵ of Formula IV is aryl.

In one embodiment R⁶ of Formulae I, II, III, IV, V and VI is H, OH, halo, CN, (C₁-C₃)alkyl, (C₁-C₃)haloalkyl or (C₁-C₃)alkoxy.

In one embodiment R⁷ of Formulae I, II, III, IV, V and VI is H, (C₁-C₈)alkyl or (C₁-C₈)haloalkyl.

In one embodiment R⁸ of Formulae I, II, III, IV, V and VI is CO₂H or C(O)N(H)SO₂R⁵.

In one embodiment R⁹ of Formula III is (C₁-C₈)alkyl or (C₁-C₈)haloalkyl.

In one embodiment R⁹ of Formula III is cycloalkyl or heterocyclyl.

In one embodiment “m” of Formulae I and II=2 or 3. In another embodiment “n” of Formulae I, II, IV, V and VI=1 or 2. In yet another embodiment “p” of Formulae I, II, III, IV, V and VI=0 or 1.

In one embodiment the optional substituents of alkyl, cycloalkyl, heterocyclyl, heteroaryl or aryl are OH, CN, halogen, (C₁-C₈)alkyl, O(C₁-C₈)alkyl, haloalkyl, alkylene-C(O)NH₂ or alkylene-C(O)—NH(Me).

In one embodiment the optional substituents of alkyl, cycloalkyl, heterocyclyl, heteroaryl or aryl are cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

In certain embodiments, an eIF4E inhibitor is compound X according to:

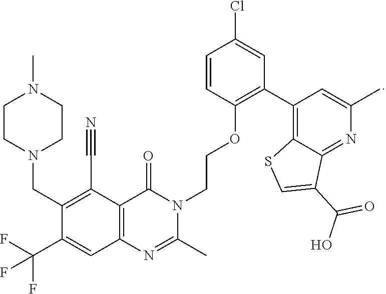 [SMILES: Cc1cc(-c2cc(Cl)ccc2OCCn2c(C)nc3cc(C(F)(F)F)c(CN4CCN(C)CC4)c(C#N)c3c2=O)c2scc(C(=O)O)c2n1]

In certain embodiments, an eIF4E inhibitor is compound Y according to:

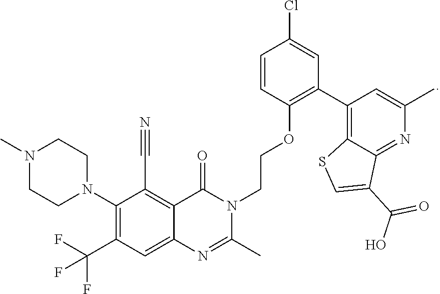

In yet further embodiments, an eIF4E inhibitor is selected from
7-(5-chloro-2-(2-(5-cyano-2-methyl-4-oxo-7-(trifluoromethyl)quinazolin-3(4H)-yl)ethoxy)phenyl)thieno[3,2-b]pyridine-3-carboxylic acid,
7-(5-chloro-2-(2-(5-cyano-6-(4,4-difluorocyclohexyl)-2-methyl-4-oxopyrido[3,4-d]pyrimidin-3(4H)-yl)ethoxy)phenyl)thieno[3,2-b]pyridine-3-carboxylic acid,
7-(5-chloro-2-(2-(5-cyano-6-((dimethylamino)methyl)-2-methyl-4-oxo-7-(trifluoromethyl)quinazolin-3(4H)-yl)ethoxy)phenyl)thieno[3-2-b]pyridine-3-carboxylic acid,
7-(5-chloro-2-(2-(5-cyano-2-methyl-4-oxo-7-(trifluoromethyl)quinazolin-3(4H)-yl)ethoxy)phenyl)-5-methylthieno[3,2-b]pyridine-3-carboxylic acid,
5′-chloro-2′-(2-(5-cyano-2-methyl-6-(4-methylpiperazin-1-yl)-4-oxo-7-(trifluoromethyl)quinazolin-3(4H)-yl)ethoxy)-[1,1′-biphenyl]-3-carboxylic acid,
7-(5-chloro-2-(2-(2-methyl-6-(4-methylpiperazin-1-yl)-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-6-(difluoromethoxy)-7-((dimethylamino)methyl)-2-methyl-4-oxoquinazolin-3(4H)-yl)ethoxy)phenyl)thieno[3,2-b]pyridine-3-carboxylic acid,
7-(5-chloro-2-(2-(5-cyano-6-(5-fluoro-2-methylpyridin-3-yl)-2-methyl-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-2-methyl-6-((4-methylpiperazin-1-yl)methyl)-4-oxo-7-(trifluoromethyl)quinazolin-3(4H)-yl)ethoxy)phenyl)-5-methylthieno[3,2-b]pyridine-3-carboxylic acid,
7-(5-Chloro-2-(2-(5-cyano-6-((dimethylamino)methyl)-2-methyl-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-6-(2-(dimethylamino)ethyl)-2-methyl-4-oxoquinazolin-3(4H)-yl)ethoxy)phenyl)-5-methylthieno[3,2-b]pyridine-3-carboxylic acid,
7-(5-Chloro-2-(2-(5-cyano-6-(2-(dimethylamino)ethyl)-2-methyl-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(2-methyl-6-(4-methylpiperazin-1-yl)-4-oxo-7-(trifluoromethyl)pyrido[3,2-d]pyrimidin-3(4H)-yl)ethoxy)phenyl)-5-methylthieno[3,2-b]pyridine-3-carboxylic acid,
3-(2-(4-chloro-2-(thieno[3,2-b]pyridin-7-yl)phenoxy)ethyl)-2-methyl-6-(4-methylpiperazin-1-yl)-4-oxo-7-(trifluoromethyl)-3,4-dihydroquinazoline-5-carbonitrile,
7-(5-chloro-2-(2-(5-cyano-2-methyl-6-(4-methylpiperazin-1-yl)-4-oxopyrido[3,4-d]pyrimidin-3(4H)-yl)ethoxy)phenyl)thieno[3,2-b]pyridine-3-carboxylic acid,
7-(5-chloro-2-(2-(5-cyano-2-methyl-6-(1-methylcyclopropyl)-4-oxopyrido[3,4-d]pyrimidin-3 (4H)-yl)ethoxy)phenyl)-5-methylthieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3 (4H)-yl)ethoxy)phenyl)thieno[3,2-b]pyridine-3-carboxylic acid,
3-(2-(4-chloro-2-(5-methylthieno[3,2-b]pyridin-7-yl)phenoxy)ethyl)-2-methyl-6-(4-methylpiperazin-1-yl)-4-oxo-7-(trifluoromethyl)-3,4-dihydroquinazoline-5-carbonitrile,
7-(5-chloro-2-(2-(5-cyano-2-methyl-4-oxo-6-(1-(trifluoromethyl)cyclopropyl)pyrido[3,4-d]pyrimidin-3 (4H)-yl)ethoxy)phenyl)-5-methylthieno[3,2-b]pyridine-3-carboxylic acid
7-(5-chloro-2-(3-(5-cyano-2-methyl-4-oxo-6-(4-(2,2,2-trifluoroethyl)piperazin-1-yl)pyrido[3,4-d]pyrimidin-3 (4H)-yl)prop-1-yn-1-yl)phenyl)thieno[3,2-b]pyridine-3-carboxylic acid,
7-(5-chloro-2-(2-(5-cyano-6-(4,4-difluorocyclohex-1-en-1-yl)-2-methyl-4-oxopyrido[3,4-d]pyrimidin-3 (4H)-yl)ethoxy)phenyl)thieno[3,2-b]pyridine-3-carboxylic acid,
7-(5-chloro-2-(2-(5-cyano-6-(3-(2,2-difluoroethoxy)azetidin-1-yl)-2-methyl-4-oxopyrido[3,4-d]pyrimidin-3 (4H)-yl)ethoxy)phenyl)-5-methylthieno[3,2-b]pyridine-3-carboxylic acid,
7-(5-chloro-2-(2-(5-cyano-6-ethyl-2-methyl-4-oxopyrido[3,4-d]pyrimidin-3 (4H)-yl)ethoxy)phenyl)-5-ethylthieno[3,2-b]pyridine-3-carboxylic acid,
7-(5-Chloro-2-(2-(5-cyano-2-methyl-6-(4-methylpiperazin-1-yl)-4-oxo-7-(trifluoromethyl)quinazolin-3 (4H)-yl)ethoxy)phenyl)-5-ethylthieno[3,2-b]pyridine-3-carboxylic acid,
7-(5-Chloro-2-(2-(5-cyano-2-methyl-6-(4-methylpiperazin-1-yl)-4-oxo-7-(trifluoromethyl)quinazolin-3 (4H)-yl)ethoxy)phenyl)-2-methylthieno[3,2-b]pyridine-3-carboxylic acid,
7-(5-chloro-2-(3-(5-cyano-6-((trans-4-(3,3-difluoroazetidin-1-yl)cyclohexyl)(methyl)amino)-2-methyl-4-oxopyrido[3,4-d]pyrimidin-3 (4H)-yl)prop-1-yn-1-yl)phenyl)thieno[3,2-b]pyridine-3-carboxylic acid,
7-(5-chloro-2-(3-(5-cyano-2-methyl-6-(methyl (1-(2,2,3,3-tetrafluoropropyl)piperidin-4-yl)amino)-4-oxopyrido[3,4-d]pyrimidin-3 (4H)-yl)prop-1-yn-1-yl)phenyl)thieno[3,2-b]pyridine-3-carboxylic acid, γ-(5-chloro-2-(3-(5-cyano-6-((1-(2,2-difluoroethyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2,2-difluoro-3-hydroxy-3-methylbutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-((1r,3r)-3-(difluoromethoxy)cyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(2-(2,2-difluoroethyl)-2,7-diazaspiro[3.5]nonan-7-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(2-(2,2-difluoropropyl)-2,7-diazaspiro[3.5]nonan-7-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(2-(5-cyano-6-(2-(dimethylamino)ethyl)-2-methyl-4-oxoquinazolin-3(4H)-yl)ethoxy)phenyl)-5-methyl-N-(methylsulfonyl)thieno[3,2-b]pyridine-3-carboxamide,
7-(5-chloro-2-(2-(5-cyano-6-((1-(2,2-difluoropropyl)piperidin-4-yl)(methyl)amino)-2-methyl-4-oxopyrido[3,4-d]pyrimidin-3(4H)-yl)ethoxy)phenyl)thieno[3,2-b]pyridine-3-carboxylic acid,
7-(5-chloro-2-(3-(5-cyano-2-methyl-6-(methyl(1-(2,2,2-trifluoroethyl)piperidin-4-yl)amino)-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2,2-difluoropropyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2-fluoro-2-methylpropyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2,2-difluoropropyl)piperidin-4-yl)(methyl)amino)-2-methyl-4-oxopyrido[3,4-d]pyrimidin-3 (4H)-yl)prop-1-yn-1-yl)phenyl)-N-(pyridin-4-ylsulfonyl)thieno[3,2-b]pyridine-3-carboxamide,
7-(5-chloro-2-(3-(5-cyano-6-((1-(2,2-difluoropropyl)piperidin-4-yl)(methyl)amino)-2-methyl-4-oxopyrido[3,4-d]pyrimidin-3 (4H)-yl)prop-1-yn-1-yl)phenyl)-N-(pyridin-3-yl sulfonyl)thieno[3,2-b]pyridine-3-carboxamide,
7-(5-chloro-2-(2-(5-cyano-2-methyl-6-(4-methylpiperazin-1-yl)-4-oxopyrido[3,4-d]pyrimidin-3 (4H)-yl)ethoxy)phenyl)-5-methylthieno[3,2-b]pyridine-3-carboxylic acid
7-(5-chloro-2-(2-(5-cyano-2,8-dimethyl-4-oxo-6-(2-(trifluoromethyl)phenyl)pyrido[3,4-d]pyrimidin-3 (4H)-yl)ethoxy)phenyl)thieno[3,2-b]pyridine-3-carboxylic acid,
7-(5-chloro-2-(2-(5-cyano-6-(3-hydroxypyrrolidin-1-yl)-2,8-dimethyl-4-oxopyrido[3,4-d]pyrimidin-3 (4H)-yl)ethoxy)phenyl)-5-methylthieno[3,2-b]pyridine-3-carboxylic acid
7-(5-Chloro-2-(2-(5-cyano-2-methyl-6-(4-methylpiperazin-1-yl)-7-(methylsulfonyl)-4-oxoquinazolin-3 (4H)-yl)ethoxy)phenyl)-2,5-dimethylthieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3 (4H)-yl)ethoxy)phenyl)-N-(methylsulfonyl)thieno[3,2-b]pyridine-3-carboxamide,
7-(5-chloro-2-(2-(5-cyano-6-((1 s,3 s)-3-methoxycyclobutyl)-2-methyl-4-oxopyrido[3,4-d]pyrimidin-3 (4H)-yl)ethoxy)phenyl)-5-methylthieno[3,2-b]pyridine-3-carboxylic acid,
7-(5-chloro-2-(2-(5-cyano-2-methyl-4-oxo-6-(2,2,2-trifluoroethoxy)-7-(trifluoromethyl)quinazolin-3 (4H)-yl)ethoxy)phenyl)thieno[3,2-b]pyridine-3-carboxylic acid,
5′-Chloro-2'-(3-(5-cyano-2-methyl-6-(4-methylpiperazin-1-yl)-4-oxo-7-(trifluoromethyl)quinazolin-3 (4H)-yl)prop-1-yn-1-yl)-[1,1′-biphenyl]-3-carboxylic acid,
7-(5-chloro-2-(2-(2-methyl-6-(4-methylpiperazin-1-yl)-4-oxo-7-(trifluoromethyl)pyrido[3,2-d]pyrimidin-3 (4H)-yl)ethoxy)phenyl)-N-(methylsulfonyl)thieno[3,2-b]pyridine-3-carboxamide,
7-(5-chloro-2-(2-(5-cyano-2-methyl-4-oxo-7-(trifluoromethyl)-6-(4-(3,3,3-trifluoropropyl)piperazin-1-yl)quinazolin-3 (4H)-yl)ethoxy)phenyl)thieno[3,2-b]pyridine-3-carboxylic acid,
7-(5-chloro-2-(2-(5-cyano-2-methyl-6-(methyl(1-(2,2,2-trifluoroethyl)piperidin-4-yl)amino)-4-oxopyrido[3,4-d]pyrimidin-3(4H)-yl)ethoxy)phenyl)-N-(methylsulfonyl)thieno[3,2-b]pyridine-3-carboxamide,
7-(5-chloro-2-(2-(5-cyano-6-(6-cyclopropyl-2,6-diazaspiro[3.3]heptan-2-yl)-2-methyl-4-oxo-7-(trifluoromethyl)quinazolin-3(4H)-yl)ethoxy)phenyl)thieno[3,2-b]pyridine-3-carboxylic acid,
7-(5-Chloro-2-(2-(5-cyano-6-(4-cyclopropylpiperazin-1-yl)-2-methyl-4-oxo-7-(trifluoromethyl)quinazolin-3(4H)-yl)ethoxy)phenyl)thieno[3,2-b]pyridine-3-carboxylic acid,
7-(5-chloro-2-(3-(5-cyano-2-methyl-4-oxo-6-(4-(3,3,3-trifluoropropyl)piperazin-1-yl)pyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-2-methyl-4-oxo-6-(4-(2-(trifluoromethoxy)ethyl)piperazin-1-yl)pyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(4-cyclopropylpiperazin-1-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(4-(3,3-difluorocyclobutyl)piperazin-1-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(2-(5-cyano-6-(4-(2,3-difluoro-2-methylpropyl)piperazin-1-yl)-2-methyl-4-oxopyrido[3,4-d]pyrimidin-3(4H)-yl)ethoxy)phenyl)thieno[3,2-b]pyridine-3-carboxylic acid,
7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2,2-difluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(4-((1-fluorocyclopropyl)methyl)piperazin-1-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(2-(5-cyano-6-((1-(2,2-difluorobutyl)piperidin-4-yl)(methyl)amino)-2-methyl-4-oxopyrido[3,4-d]pyrimidin-3(4H)-yl)ethoxy)phenyl)thieno[3,2-b]pyridine-3-carboxylic acid,
7-(5-chloro-2-(3-(5-cyano-6-((1-((1-fluorocyclopropyl)methyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-2-methyl-6-(methyl(1-(2-(trifluoromethoxy)ethyl)piperidin-4-yl)amino)-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-((1-cyclopropylpiperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(3-(difluoromethoxy)cyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-2-methyl-6-(methyl(1-((1 s,3 s)-3-(trifluoromethoxy)cyclobutyl)piperidin-4-yl)amino)-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-((1-fluorocyclobutyl)methyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-2-methyl-6-(methyl(1-((1R,2R)-2-(trifluoromethyl)cyclopropyl)piperidin-4-yl)amino)-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-(ethyl(1-(2,2,2-trifluoroethyl)piperidin-4-yl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-((1-(2,2-difluoropropyl)piperidin-4-yl)(ethyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(6-cyclopropyl-2,6-diazaspiro[3.3]heptan-2-yl)-2-methyl-4-oxo-7-(trifluoromethyl)quinazolin-3(4H)-yl)prop-1-yn-1-yl)phenyl)thieno[3,2-b]pyridine-3-carboxylic acid,
7-(5-chloro-2-(2-(5-cyano-6-(4-cyclopropylpiperazin-1-yl)-2-methyl-4-oxo-7-(trifluoromethyl)quinazolin-3(4H)-yl)ethoxy)phenyl)-N-(methylsulfonyl)thieno[3,2-b]pyridine-3-carboxamide,
7-(5-Chloro-2-(3-(5-cyano-6-(4,4-difluoro-[1,4'-bipiperidin]-1′-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-((1r,3r)-3-fluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-((1-(3-(difluoromethyl)oxetan-3-yl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(2,2-difluoropropyl)piperidin-4-yl)(methyl)amino)-2-methyl-4-oxopyrido[3,4-d]pyrimidin-3(4H)-yl)prop-1-yn-1-yl)phenyl)-N-(oxetan-3-ylsulfonyl)thieno[3,2-b]pyridine-3-carboxamide,
7-(5-chloro-2-(3-(5-cyano-6-((1-(3,3-difluorocyclobutyl)piperidin-4-yl)(ethyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-((3-fluorooxetan-3-yl)methyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-(ethyl(1-(oxetan-3-yl)piperidin-4-yl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-(4-(3-(difluoromethoxy)azetidin-1-yl)piperidin-1-yl)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-(methylsulfonyl)thieno[3,2-b]pyridine-3-carboxamide,
7-(5-Chloro-2-(3-(5-cyano-6-((1-((3-(difluoromethoxy)cyclobutyl)methyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-chloro-2-(3-(5-cyano-6-((1-(3,3-difluorobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-6-(ethyl(1-(2,2,3,3-tetrafluoropropyl)piperidin-4-yl)amino)-2-methyl-4-oxopyrido[3,4-d]pyrimidin-3(4H)-yl)prop-1-yn-1-yl)phenyl)thieno[3,2-b]pyridine-3-carboxylic acid,
7-(5-Chloro-2-(3-(5-cyano-2-methyl-6-(4-methylpiperazin-1-yl)-4-oxo-7-(trifluoromethyl)quinazolin-3(4H)-yl)prop-1-yn-1-yl)phenyl)thieno[3,2-b]pyridine-3-carboxylic acid,
7-(5-Chloro-2-(3-(5-cyano-2-methyl-6-(4-methylpiperazin-1-yl)-4-oxo-7-(trifluoromethyl)quinazolin-3(4H)-yl)prop-1-yn-1-yl)phenyl)-5-methylthieno[3,2-b]pyridine-3-carboxylic acid,
7-(5-chloro-2-(2-(5-cyano-6-(4-(2,2-difluoroethyl)piperazin-1-yl)-2-methyl-4-oxo-7-(trifluoromethyl)quinazolin-3(4H)-yl)ethoxy)phenyl)thieno[3,2-b]pyridine-3-carboxylic
7-(5-chloro-2-(2-(5-cyano-6-((1-(2-cyclopropyl-2,2-difluoroethyl)piperidin-4-yl)(methyl)amino)-2-methyl-4-oxopyrido[3,4-d]pyrimidin-3(4H)-yl)ethoxy)phenyl)thieno[3,2-b]pyridine-3-carboxylic acid,
7-(5-chloro-2-(3-(5-cyano-2-methyl-6-(methyl (1-(3-methyl oxetan-3-yl)piperidin-4-yl)amino)-4-oxopyrido[3,4-d]pyrimidin-3(4H)-yl)prop-1-yn-1-yl)phenyl)thieno[3,2-b]pyridine-3-carboxylic acid,
Methyl 7-(5-chloro-2-(3-(5-cyano-2-methyl-4-oxo-6-(4-(2-(trifluoromethoxy)ethyl)piperazin-1-yl)pyrido[3,4-d]pyrimidin-3(4H)-yl)prop-1-yn-1-yl)phenyl)thieno[3,2-b]pyridine-3-carboxylate,
Methyl 7-(5-chloro-2-(3-(5-cyano-6-((1-(2,2-difluoroethyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylate,
Methyl 7-(5-chloro-2-(3-(5-cyano-2-methyl-6-(methyl(1-(2,2,2-trifluoroethyl)piperidin-4-yl)amino)-4-oxopyrido[3,4-d]pyrimidin-3(4H)-yl)prop-1-yn-1-yl)phenyl)thieno[3,2-b]pyridine-3-carboxylate,
Methyl 7-(5-chloro-2-(2-(5-cyano-6-(4-cyclopropylpiperazin-1-yl)-2-methyl-4-oxo-7-(trifluoromethyl)quinazolin-3(4H)-yl)ethoxy)phenyl)thieno[3,2-b]pyridine-3-carboxylate,
7-(5-chloro-2-(2-(5-cyano-2,8-dimethyl-4-oxo-6-(4-(2,2,2-trifluoroethyl)piperazin-1-yl)pyrido[3,4-d]pyrimidin-3(4H)-yl)ethoxy)phenyl)thieno[3,2-b]pyridine-3-carboxylic acid
7-(5-chloro-2-(2-(5-cyano-6-(4-(2-fluoroethyl)piperazin-1-yl)-2-methyl-4-oxopyrido[3,4-d]pyrimidin-3(4H)-yl)ethoxy)phenyl)thieno[3,2-b]pyridine-3-carboxylic acid,
7-(5-chloro-2-(2-(5-cyano-6-(4-(2,2-difluoroethyl)piperazin-1-yl)-2-methyl-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-6-(4-(2-fluoroethyl)piperazin-1-yl)-2-methyl-4-oxo-7-(trifluoromethyl)quinazolin-3(4H)-yl)ethoxy)phenyl)-5-methylthieno[3,2-b]pyridine-3-carboxylic acid,
7-(5-Chloro-2-(2-(5-cyano-2-methyl-6-(4-methylpiperazin-1-yl)-4-oxo-7-(trifluoromethyl)quinazolin-3(4H)-yl)ethoxy)phenyl)-5-ethylthieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3(4H)-yl)ethoxy)phenyl)-5-ethyl-2-methylthieno[3,2-b]pyridine-3-carboxylic acid,
7-(5-chloro-2-(2-(5-cyano-6-(4-(2-methoxyethyl)piperazin-1-yl)-2-methyl-4-oxo-7-(trifluoromethyl)quinazolin-3(4H)-yl)ethoxy)phenyl)thieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3(4H)-yl)ethoxy)phenyl)-5-(fluoromethyl)-2-methylthieno[3,2-b]pyridine-3-carboxylic acid,
7-(5-Chloro-2-(2-(5-cyano-2-methyl-6-(4-(oxetan-3-yl)piperazin-1-yl)-4-oxo-7-(trifluoromethyl)quinazolin-3(4H)-yl)ethoxy)phenyl)thieno[3,2-b]pyridine-3-carboxylic acid,
7-(5-Chloro-2-(2-(5-cyano-2-methyl-6-(4-(oxetan-3-yl)piperazin-1-yl)-4-oxo-7-(trifluoromethyl)quinazolin-3(4H)-yl)ethoxy)phenyl)-5-methylthieno[3,2-b]pyridine-3-carboxylic acid,
7-(5-chloro-2-(2-(5-cyano-2-methyl-6-(4-methylpiperazin-1-yl)-4-oxo-7-(trifluoromethyl)quinazolin-3(4H)-yl)ethoxy)phenyl)-5-(methoxymethyl)-2-methylthieno[3,2-b]pyridine-3-carboxylic acid, and
7-(5-chloro-2-(3-(5-cyano-6-((trans-4-(3,3-difluoroazetidin-1-yl)cyclohexyl)(methyl)amino)-2-methyl-4-oxopyrido[3,4-d]pyrimidin-3(4H)-yl)prop-1-yn-1-yl)phenyl)-N-(methylsulfonyl)thieno[3,2-b]pyridine-3-carboxamide, or

any combination of two to four of the compounds.

In further embodiments, an eIF4E inhibitor is an antisense oligonucleotide. Examples of eIF4E specific antisense oligonucleotides are described in PCT Publication No. WO 2005/028628, the inhibitors of which are incorporated herein by reference in their entirety.

Methods of measuring inhibition of eIF4E binding eIF4G include an m⁷GTP pull-down assay (Moerke et al., Cell 128:257-267, 2007, which assay is incorporated herein by reference in its entirety); fluorescence polarization competition assay (Moerke et al., 2007; PCT Publication No. WO 2014/149001; each assay of which is incorporated herein by reference in its entirety), and a cell based assay comprising Gaussia luciferase reporter gene with a 5′-UTR of c-myc (PCT Publication No. WO 2011/136744, the assay of which is incorporated herein by reference in its entirety). Methods of measuring inhibition of eIF4E binding to the mRNA cap include fluorescence polarization competition assay (U.S. application Ser. No. 16/916,820 (claiming priority to U.S. Provisional Application No. 62/869,662)), which assays are incorporated herein by reference in their entirety) and competition binding assay involving cross-linking of recombinant eIF4E to cap-labeled oxidized mRNA (Sonenberg et al., Proc. Nat'l. Acad. Sci. U.S.A. 74:4288-4292, 1977; Sonenberg et al., Proc. Nat'l. Acad. Sci. U.S.A. 75:4843-4847, 1978, the assays of which are incorporated herein by reference in their entirety).

### C. Combination Therapy

In other aspects, methods of the present disclosure provide administering to the subject having BRAF-mutated cancer cells an additional therapeutic agent. For example, a combination therapy may comprise administering an eIF4E inhibitor in combination with an inhibitor of an immunosuppression component, radiation therapy, surgery, a chemotherapeutic agent (e.g., a RAF inhibitor, MEK inhibitor, mTOR inhibitor, MNK specific inhibitor, eIF4A inhibitor, or any combination thereof), an immunotherapeutic agent targeting an cancer antigen expressed by the tumor (e.g., antibody or adoptive immunotherapeutic agent), a cytokine, an RNA interference agent, or any combination thereof, which components may be administered simultaneously, concurrently, or sequentially.

As used herein, a “chemotherapeutic agent” includes to traditional cytotoxic agents that inhibits cell growth, inhibits cell proliferation, leads to cell death or the like in rapidly dividing cells, as well as targeted, cytostatic agents that inhibit a target molecule involved in carcinogenesis and tumor growth.

A chemotherapeutic agent includes, for example, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), or a DNA repair inhibitor. Chemotherapeutic agents include, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (methotrexate, pemetrexed, mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, vindesine, vinorelbine, nocodazole, epothilones, eribulin and navelbine; epidipodophyllotoxins (etoposide, teniposide); DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); DNA methyltransferase inhibitors (azacytidine); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkylsulfonates (busulfan), nitrosoureas (carmustine (BCNU) and analogs, streptozocin), triazenes (dacarbazine (DTIC)); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein, pomalidomide) and growth factor inhibitors (vascular endothelial growth factor (VEGF)) inhibitors, such as ziv-aflibercept; fibroblast growth factor (FGF) inhibitors); inhibitors of apoptosis protein (TAP) antagonists (birinapant); histone deacetylase (HDAC) inhibitors (vorinostat, romidepsin, chidamide, panobinostat, mocetinostat, abexinostat, belinostat, entinostat, resminostat, givinostat, quisinostat, SB939); proteasome inhibitors (ixazomib); angiotensin receptor blocker; nitric oxide donors; anti-sense oligonucleotides; antibodies (trastuzumab, panitumumab, pertuzumab, cetuximab, adalimumab, golimumab, infliximab, rituximab, ocrelizumab, ofatumumab, obinutuzumab, alemtuzumab, abciximab, atlizumab, daclizumab, denosumab, efalizumab, elotuzumab, rovelizumab, ruplizumab, ustekinumab, visilizumab, gemtuzumab ozogamicin, brentuximb vedotin); chimeric antigen receptors; cell cycle inhibitors (flavopiridol, roscovitine, bryostatin-1) and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); PARP inhibitors (niraparib, olaparib); focal adhesion kinase (FAK) inhibitors (defactinib (VS-6063), VS-4718, VS-6062, GSK2256098); growth factor signal transduction kinase inhibitors (cediranib, galunisertib, rociletinib, vandetanib, afatinib, EGF816, AZD4547); c-Met inhibitors (capmatinib, INC280); tyrosine kinase inhibitors; serine/threonine kinase inhibitors; ALK inhibitors (ceritinib, crizotinib); mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

As used herein, the term “vascular endothelial growth factor inhibitor” or “VEGF inhibitor” refers to any agent that reduces or inhibits the activity of VEGF. VEGF is a pro-angiogenic factor that promotes vasculogenesis, angiogenesis, and increases vascular permeability. VEGF may refer to VEGF-A, VEGF-B, VEGF-C, VEGF-D, VEGF-E, or any combination thereof. Non-limiting examples of VEGF inhibitors include bevacizumab, ranibizumab, AZD2171, cannbidiol, THC, or any combination thereof.

As used herein, the term “vascular endothelial growth factor receptor inhibitor” or “VEGFR inhibitor” refers to any agent that inhibits the activity of VEGF-specific tyrosine kinase receptors VEGFR1, VEGFR2, VEGFR3, or any combination thereof. Non-limiting examples of VEGFR inhibitors include axitinib, sunitinib, vatalanib, sorafenib, GW-786034, CP-547632, AG-013736, lenvatinib, motesanib, pazopanib, regorafenib, ramucirumab, CDP-791, or any combination thereof.

As used herein, the term “tyrosine kinase inhibitor” refers to any agent that inhibits a tyrosine kinase. Tyrosine kinase inhibitors include inhibitors that provide competitive ATP inhibition at the catalytic binding site of tyrosine kinase and allosteric inhibitors. Non-limiting examples of tyrosine kinase inhibitors include axitinib, imatinib, gefitinib, erlotinib, lapatinib, sorafenib, sunitinib, pazopanib, vandetanib, and dasatinib.

In certain embodiments, the subject is administered an eIF4E inhibitor in combination with a chemotherapeutic agent comprising a RAF inhibitor, MEK inhibitor, mTOR inhibitor, MNK specific inhibitor, eIF4A inhibitor, or any combination thereof.

### (i) MNK-Specific Inhibitors

A “MNK inhibitor,” as used herein, may directly block, inactivate, reduce or minimize MNK activity (e.g., kinase activity or translational effects), or reduce activity by promoting degradation of MNK, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated MNK. In certain embodiments, a MNK inhibitor blocks, inactivates, reduces or minimizes the ability of MNK to phosphorylate eIF4E, hnRNPA1, PSF or combinations thereof. In further embodiments, a MNK inhibitor enhances or promotes expansion of CD4+ central memory T cells, CD8+ central memory T cells, or both. In yet further embodiments, a MNK inhibitor induces or enhances a T cell response. Non-limiting examples of inhibitors include small molecules, antisense molecules, ribozymes, inhibitory nucleic acid molecules, endonucleases, or the like.

As used herein, a “MNK-specific inhibitor” refers to an agent that (a) inhibits MNK enzyme (kinase) activity (i.e., MNK1 and MNK2), (b) has at least about 25-fold less activity against the rest of a host cell kinome as set forth in Table A (i.e., other than MNK enzymes), and (c) does not significantly reduce or inhibit IL-2 production by T cells. As used herein, “a host cell kinome” refers to the 412 protein and lipid kinases listed in Table A (not including the MNK1 and MNK2 enzymes), which may be from a particular organism or cell of interest (e.g., human). The activity of a host cell kinome in the presence and absence of a candidate MNK-specific inhibitor or a known MNK-specific inhibitor (see, e.g., Compound 107 of Table B) is measured using the FRET-based method of Rodems et al. (Assay. Drug Dev. Technol. 1:9, 2002, which assay is incorporated herein by reference in its entirety).

In certain embodiments, the host cell kinome of Table A is from a human cell. In further embodiments, a MNK-specific inhibitor compound is a small molecule and has at least 50-fold less activity against a serine/threonine kinome of an organism or cell as listed in Table A, and does not significantly reduce or inhibit IL-2 production by T cells. In particular embodiments, the serine/threonine kinome of Table A is from a human cell. In still further embodiments, a MNK-specific inhibitor compound has at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold less, 200-fold less, 250-fold less, 300-fold less, 400-fold less, 500-fold less, 750-fold less, 1000-fold less, or even less activity against kinome enzymes of Table A other than the serine/threonine kinome enzymes of Table A, and does not significantly reduce or inhibit IL-2 production by T cells.

TABLE A
Protein and Lipid Kinases of “Host Cell Kinome” (excluding MNK)
Kinome Kinases

| | | | |
|---|---|---|---|
| STK17A (DRAK1) | CAMK2D | STK4 (MST1) | MATK (HYL) |
| | (CaMKIIδ) | | |
| CLK4 | MAP2K1 (MEK1) | ABL2 (Arg) | PAK2 (PAK65) |
| | S218D S222D | | |
| LRRK2 G2019S | KIT T670I | EPHB1 | MAP2K2 (MEK2) |
| LRRK2 R1441C | SNF1LK2 | MAP3K2 (MEKK2) | HIPK1 (Myak) |
| LRRK2 G2019S FL | LATS2 | PDK1 Direct | PRKX |
| FLT3 D835Y | MAPK3 (ERK1) | PRKCQ (PKC theta) | MAP2K6 (MKK6) |
| | | | S207E T211E |
| TGFBR2 | TLK2 | DDR2 T654M | SIK1 |
| PDGFRA V561D | PI4KB (PI4Kβ) | CSNK2A1 (CK2α1) | CDK2/cyclin A1 |
| LRRK2 FL | RAF1 (cRAF) | PRKCG (PKCγ) | EPHB3 |
| | Y340D Y341D | | |
| LRRK2 | MAPK14 (p38α) | EGFR (ErbB1) | EIF2AK2 (PKR) |
| | | d746-750 | |
| BRSK1 (SAD1) | NTRK3 (TRKC) | PRKCI (PKC iota) | SGK (SGK1) |
| STK17B (DRAK2) | EEF2K | RET V804M | GRK5 |
| RIPK2 | RPS6KA5 (MSK1) | AXL | CAMK2B |
| | | | (CaMKIIβ) |
| TNIK | CSF1R (FMS) | PLK1 | ALK C1156Y |
| LRRK2 I2020T | CSNK1D (CK1δ) | CHEK1 (CHK1) | JAK3 |
| KDR (VEGFR2) | ABL1 M351T | STK32C (YANK3) | MYLK (MLCK) |
| PDGFRA D842V | CSNK1G3 (CK1γ3) | HIPK2 | TAOK3 (JIK) |
| KIT D816V | ACVR1 (ALK2) | TEK (TIE2) Y1108F | MAP2K3 (MEK3) |
| | R206H | | |
| KIT A829P | BRAF | MST1R (RON) | WNK3 |
| RET | CDC7/DBF4 | ULK1 | KIT V654A |
| DYRK3 | MAPK13 (p38δ) | PRKCH (PKC eta) | GRK7 |
| DYRK2 | CDC42 BPA | STK22D (TSSK1) | CSNK1A1 (CK1 α |
| | (MRCKA) | | 1) |
| RPS6KA6 (RSK4) | KIT N822K | FGFR3 G697C | CDK9 (Inactive) |
| MINK1 | CAMK1D | LIMK1 | EGFR (ErbB1) |
| | (CaMKIδ) | | T790M |
| MAP3K8 (COT) | MAP2K6 (MKK6) | STK22B (TSSK2) | TEC |
| RET Y791F | PIK3CD/PIK3R1 | MAP3K10 (MLK2) | MAP4K3 (GLK) |
| | (p110δ/p85α) | | |
| BRAF V599E | CLK3 | MAPK10 (JNK3) | MAP3K14 (NIK) |
| RET V804L | EPHA2 | PHKG1 | AMPK (A2/B2/G1) |
| BMPR2 | MAPKAPK3 | NLK | TYK2 |
| PRKG2 (PKG2) | MST4 | KIT | JAK1 |
| MAPK9 (JNK2) | STK25 (YSK1) | BRAF | ACVRL1 (ALK1) |
| KIT D816H | FGFR1 | MAP4K2 (GCK) | MAP4K4 (HGK) |
| PRKD1 (PKC mu) | CSNK1E (CK1ζ) | PIK3CG (p110γ) | DMPK |
| DYRK1A | TYRO3 (RSE) | MET M1250T | MAPK9 (JNK2) |
| CAMK4 (CaMKIV) | FLT3 ITD | CSNK2A2 (CK2α2) | TNK2 (ACK) |
| STK24 (MST3) | PLK2 | TAOK1 | PKN2 (PRK2) |
| PAK7 (KIAA1264) | EPHA7 | ABL1 | PRKG1 |
| AURKC (Aurora C) | CDK1/cyclin B | CDK2/cyclin A2 | LTK (TYK1) |
| ZAP70 | AKT2 (PKBβ) | TEK (TIE2) R849W | CDK7/cyclin |
| | | | H/MNAT1 |
| MAP2K2 (MEK2) | CDK5/p35 | NUAK1 (ARK5) | ACVR1 (ALK2) |
| PRKCN (PKD3) | SRPK2 | ABL1 G250E | BMPR1A (ALK3) |
| FLT3 | INSR | PAK6 | DDR1 |
| STK39 (STLK3) | MAP2K6 (MKK6) | CDC42 BPB | ERBB4 (HER4) |
| | | (MRCKB) | |
| RET G691S | MARK2 | CDK9/cyclin K | CDK16 |
| | | | (PCTK1)/cyclin Y |
| AURKB (Aurora B) | CLK1 | CAMK2A | AMPK (A1/B1/G2) |
| | | (CaMKIIα) | |
| GSK3A (GSK3α) | GSG2 (Haspin) | JAK2 JH1 JH2 | MAP2K1 (MEK1) |
| | | V617F | |
| MAPK8 (JNK1) | EPHA4 | CASK | EGFR (ErbB1) |
| | | | L858R |
| SRMS (Srm) | MAPK12 (p38γ) | ACVR2A | PTK6 (Brk) |
| PAK3 | TXK | ALK L1196M | NUAK2 |
| MAPK11 (p38β) | ABL1 Q252H | TTK | STK38L (NDR2) |
| DYRK1B | PASK | DYRK4 | ADRBK2 (GRK3) |
| DNA-PK | GRK4 | WNK2 | MAPK15 (ERK7) |
| IGF1R | FGFR3 | FLT1 (VEGFR1) | ACVR2B |
| PTK2 (FAK) | DAPK2 | PAK1 | MAP3K11 (MLK3) |
| FER | STK23 (MSSK1) | LCK | AXL R499C |
| CSNK1G1 (CK1γ1) | STK3 (MST2) | SRPK1 | PKN1 (PRK1) |
| DDR2 N456S | BRAF V599E | PHKG2 | CDK3/cyclin E1 |
| EPHA5 | AMPK A1/B1/G1 | BMPR1B (ALK6) | MAP4K1 (HPK1) |
| FGFR4 | EGFR (ErbB1) | BLK | CAMK2G |
| | L861Q | | (CaMKIIγ) |
| FGR | AKT1 (PKBα) | MARK4 | MET D1228H |
| SRC | CLK2 | PRKCB1 (PKCβ I) | WEE1 |
| MLCK (MLCK2) | ABL1 T315I | ALK F1174L | ROCK1 |
| MAPK10 (JNK3) | GRK6 | FGFR3 K650E | EPHA3 |
| MAPKAPK2 | EPHA1 | MERTK (cMER) | STK32B (YANK2) |
| | | A708S | |
| PRKD2 (PKD2) | HCK | MAP3K3 (MEKK3) | KIT Y823D |
| FRK (PTK5) | SGK2 | FGFR1 V561M | EGFR (ErbB1) |
| | | | T790M L858R |
| PDGFRA T674I | ULK2 | CDK11 (Inactive) | TAOK2 (TAO1) |
| SRC N1 | CDK5/p25 | MAP3K9 (MLK1) | IKBKE (IKKζ) |
| ROCK2 | KIT D820E | FES (FPS) | NEK9 |
| BMX | MUSK | ITK | MAPK8 (JNK1) |
| CDK2/cyclin O | PRKCA (PKCα) | ZAK | BTK |
| TBK1 | AURKA (Aurora A) | KIT T670E | AMPK (A1/B1/G3) |
| CSK | PRKACA (PKA) | ALK R1275Q | SIK3 |
| CDK1/cyclin A2 | NEK4 | LIMK2 | PIK3C3 (hVPS34) |
| HIPK4 | EPHA6 | ABL1 E255K | PIM1 |
| AMPK A2/B1/G1 | CDK8/cyclin C | MELK | FLT4 (VEGFR3) |
| EPHA8 | JAK2 JH1 JH2 | NEK2 | CDK2/cyclin E1 |
| AKT3 (PKBγ) | ALK | SLK | SPHK1 |
| YES1 | CAMKK1 | MERTK (cMER) | PDK1 |
| | (CAMKKA) | | |
| MARK3 | EPHB2 | MAP2K1 (MEK1) | EGFR (ErbB1) |
| MAPK14 (p38α) | HIPK3 (YAK1) | DDR2 | RET M918T |
| Direct | | | |
| RAF1 (cRAF) | FGFR3 K650M | INSRR (IRR) | MAP4K5 (KHS1) |
| Y340D Y341D | | | |
| IRAK4 | NTRK1 (TRKA) | TEK (Tie2) | FYN A |
| PRKCZ (PKCζ) | STK33 | MARK1 (MARK) | LATS1 |
| RPS6KA1 (RSK1) | CSNK1G2 (CK1γ2) | TLK1 | RPS6KB1 (p70S6K) |
| CAMK1 (CaMK1) | DAPK3 (ZIPK) | AMPK (A1/B2/G1) | PDGFRB |
| | | | (PDGFRβ) |
| PDGFRA | ABL1 Y253F | EPHB4 | PRKACG |
| (PDGFRα) | | | (PRKACγ) |
| RPS6KA2 (RSK3) | ROS1 | ULK3 | PLK3 |
| GSK3B (GSK3β) | MAP3K5 (ASK1) | ABL1 H396P | BRSK2 |
| PAK4 | NEK6 | CDK9/cyclin T1 | TGFBR1 (ALK5) |
| TESK2 | STK38 (NDR) | SYK | PRKCE (PKCε) |
| NEK1 | IKBKB (IKKβ) | CHEK2 (CHK2) | MAP3K7/MAP3K7I |
| | | | P1 (TAK1-TAB1) |
| DCAMKL2 (DCK2) | PRKACB | JAK2 | NEK7 |
| | (PRKACβ) | | |
| SGKL (SGK3) | MYLK2 (skMLCK) | STK16 (PKL12) | MET (cMet) |
| PIK3C2B (PI3K- | PRKCB2 (PKCβII) | PLK4 | GRK1 |
| C2β) | | | |
| CHUK (IKKα) | PIM2 | ADRBK1 (GRK2) | PIK3CA/PIK3R1 |
| | | | (p110α/p85α) |
| NTRK2 (TRKB) | CAMKK2 | AMPK (A2/B2/G2) | PIK3C2A |
| | (CaMKKβ) | | (PI3K-C2α) |
| ACVR1B (ALK4) | FRAP1 (mTOR) | MAPK1 (ERK2) | SPHK2 |
| RPS6KA3 (RSK2) | ICK | MYO3B (MYO3β) | PI4KA (PI4Kα) |
| PTK2B (FAK2) | LYN A | CDK14 | RIPK3 |
| | | (PFTK1)/cyclin Y | |
| RPS6KA4 (MSK2) | CDK2/cyclin A | DAPK1 | CDK5 (Inactive) |
| FYN | KIT V559D T670I | FGFR2 | IRAK1 |
| LYN B | MAPKAPK5 | ERBB2 (HER2) | PRKCD (PKCδ) |
| | (PRAK) | | |

In any of the aforementioned embodiments, a MNK-specific inhibitor compound can block, inactivate, reduce or minimize the ability of MNK1a, MNK1b, MNK2a, MNK2b, or any combination thereof to phosphorylate eIF4E, hnRNPA1, PSF or any combination thereof. In particular embodiments, a MNK-specific inhibitor compound can block, inactivate, reduce or minimize the ability of MNK1a, MNK1b, MNK2a, and MNK2b to phosphorylate eIF4E. MNK-specific inhibitors in any of the aforementioned embodiments may optionally not significantly reduce or inhibit (i) T cell viability, (ii) T cell proliferation, (iii) expression of MHC or HLA molecules in APCs, or (iv) production by T cells of IL-2, CD25, IFNγ or any combination thereof. Further, optionally, MNK-specific inhibitors in any of the aforementioned embodiments can also significantly reduce or inhibit expression of one or more immunosuppression components (e.g., immune checkpoint molecules, immunosuppressive cytokines) in T cells, APCs or both. The assay for measuring T cell viability is the assay described by Mosmann (J. Immunol. Meth. 65:55, 1983).

With regard to a MNK-specific inhibitor compound, “does not significantly reduce or inhibit IL-2 production by T cells” means the reduction or inhibition of IL-2 production by T cells is less than about 25%, 20%, 15%, 10%, 5%, 2%, 1%, 0.5%, 0.25%, 0.1% or less as compared to the same T cells not exposed or contacted with the MNK-specific inhibitor compound.

Also with regard to a MNK-specific inhibitor compound, “does not significantly reduce or inhibit T cells viability,” “does not significantly reduce or inhibit T cell proliferation,” “does not significantly reduce or inhibit WIC or HLA molecule expression in T cells, APCs or both,” and “does not significantly reduce or inhibit production of IL-2, CD25, IFNγ or any combination thereof by T cells,” refers to the reduction or inhibition of T cell viability; T cell proliferation; expression of WIC or HLA molecules in T cells, APCs or both; or production of IL-2, CD25, IFNγ or any combination thereof by T cells; respectively, is less than about 25%, 20%, 15%, 10%, 5%, 2%, 1%, 0.5%, 0.25%, 0.1% or less as compared to the same corresponding cells not exposed or contacted with the MNK-specific inhibitor.

Also, with regard to a MNK-specific inhibitor compound, “significantly reduce or inhibit expression of one or more immunosuppression components” means the reduction or inhibition of expression of one or more immunosuppression components in T cells, APCs or both is at least about 20%, 25%, 30%, 35%, 40%, 45%, 50%, 55%, 60%, 65%, 70% or 75% as compared to the same T cells or APCs not exposed or contacted with the MNK specific inhibitor. In certain embodiments, an APC is a cancer cell or a tumor cell.

Other assays for detecting kinase activity in the presence or absence of inhibitors are well known in the art, which can be used as a back-up to the FRET-based host cell kinome assay to show a particular MNK inhibitor compound is a MNK-specific inhibitor compound, such as the assay taught by Karaman et al. (Nat. Biotechnol. 26:127, 2007). Assays for detecting the cytokine levels (e.g., IL-2, IL-10, IFNγ) are known in the art, such as the DuoSet® ELISA assay from R&D Systems (using the manufacturer's instructions). Assays for detecting T cell viability, T cell proliferation, MHC or HLA molecule expression, and expression of immunosuppression components like immune checkpoint molecules PD-1, PD-L1, LAG3 or the like are those described in PCT Publication No. WO 2016/172010.

In certain aspects, MNK-specific inhibitor compounds that are potent and selective inhibitors of MNK1 and MNK2 may be used in the pharmaceutical compositions and methods of use described herein. MNK-specific inhibitor compounds include compounds of Formula I, Ia, IIa, IIb, IIIa, IIIb, IVa, IVb, Va, Vb, VI, Vila or VIIb, including Compound 107 (see, e.g., PCT Publication WO 2016/172010, which compounds and synthetic methods are incorporated herein in their entirety). By way of background, MNK1 and MNK2 integrate signals from several oncogenic and immune signaling pathways by phosphorylating eukaryotic initiation factor 4E (eIF4E) and other mRNA binding proteins, which regulate the stability and translation of select mRNAs important for tumor growth and survival.

Administration of a MNK-specific inhibitor to a subject in combination with the modified T cells disclosed herein may further enhance expansion of central memory T cells, enhance cytotoxic T cell activity, or both.

Exemplary MNK-specific inhibitor compounds inhibit both MNK1 and MNK2 kinase activity. In certain embodiments, a MNK-specific inhibitor selectively inhibits MNK1 kinase activity over MNK2 kinase activity, or selectively inhibits MNK2 kinase activity over MNK1 kinase activity. In other embodiments, a MNK-specific inhibitor selectively inhibits kinase activity of full length isoforms MNK1a and MNK2a over the kinase activity of MNK 1b and MNK2b. In further embodiments, a MNK-specific inhibitor selectively inhibits either MNK1 kinase activity or MNK2 kinase activity. In still further embodiments, a MNK-specific inhibitor selectively inhibits kinase activity of any one of full length isoforms MNK1a, MNK1b, MNK2a, or MNK2b, or inhibits the kinase activity of all the MNK isoforms.

In certain embodiments, a MNK-specific inhibitor compound is a compound having the following structure (I):

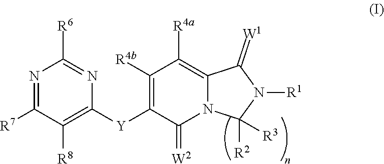

or a stereoisomer, tautomer or pharmaceutically acceptable salt thereof wherein:

W¹ and W² are independently O, S or N—OR′, where R′ is lower alkyl;

Y is —N(R⁵)—, —O—, —S—, —C(O)—, —S═O, —S(O)₂—, or —CHR⁹—;

R¹ is hydrogen, lower alkyl, cycloalkyl or heterocyclyl wherein any lower alkyl, cycloalkyl or heterocyclyl is optionally substituted with 1, 2 or 3 J groups;

n is 1, 2 or 3;

R² and R³ are each independently hydrogen, alkyl, alkenyl, alkynyl, aryl, araalkylene, heteroaryl, heteroarylalkylene, cycloalkyl, cycloalkylalkylene, heterocyclyl, or heterocyclylalkylene, wherein any alkyl, aryl, araalkylene, heteroaryl, heteroarylalkylene, cycloalkyl, cycloalkylalkylene, heterocyclyl, or heterocyclylalkylene, is optionally substituted with 1, 2 or 3 J groups;

or R² and R³ taken together with the carbon atom to which they are attached form a cycloalkyl or heterocyclyl, wherein any cycloalkyl or heterocyclyl is optionally substituted with 1, 2 or 3 J groups;

R^{4a} and R^{4b} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;

R⁵ is hydrogen, cyano, or lower alkyl;

or R⁵ and R⁸ taken together with the atoms to which they are attached form a fused heterocyclyl optionally substituted with 1, 2 or 3 J groups;

R⁶, R⁷ and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkylaminyl, heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;

or R⁷ and R⁸ taken together with the atoms to which they are attached form a fused heterocyclyl or heteroaryl optionally substituted with 1, 2 or 3 J groups;

J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl; or any two J groups bound to the same carbon or hetero atom may be taken together to form oxo; and

R⁹ is hydrogen, lower alkyl or —OH.

In certain embodiments of structure (I), the present disclosure provides a compound having the following structure (Ia), as well as stereoisomers, tautomers or pharmaceutically acceptable salts thereof:

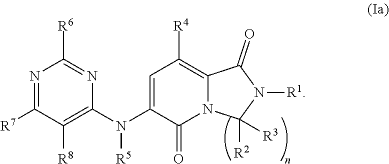

For Formula Ia compounds, substituent R¹ is hydrogen or lower alkyl and subscript n is 1, 2 or 3. Substituents R² and R³ in Formula Ia are each independently hydrogen, alkyl, cycloalkyl, cycloalkylalkylene, heterocyclyl or heterocyclylalkyl, and any such alkyl, cycloalkyl, cycloalkylalkylene, heterocyclyl or heterocyclylalkyl can optionally be substituted with 1, 2 or 3 J groups.

Substitutents R² and R³ in Formula Ia when taken together with the carbon atom to which they are attached can form a cycloalkyl or heterocyclyl, wherein any such cycloalkyl or heterocyclyl is optionally substituted with 1, 2 or 3 J groups. In Formula Ia, R^{4a} is hydrogen, halogen, hydroxy, alkyl, alkoxy, thioalkyl, alkenyl or cycloalkyl and substituent R⁵ is hydrogen or lower alkyl.

Alternatively, substituent groups R⁵ and R⁸ taken together with the atoms to which they are attached form a fused heterocyclyl that is optionally substituted with 1, 2 or 3 J groups.

In certain embodiments, substituents R⁶, R⁷ and R⁸ are independently and at each occurrence hydrogen, halogen, alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl, and any such alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl is optionally substituted with 1, 2 or 3 J groups. For some compounds in accordance with Formula Ia, R⁷ and R⁸ taken together with the atoms to which they are attached form a fused heterocyclyl unsubstituted or substituted with 1, 2 or 3 J groups.

Variable J in Formula Ia is —SH, —SR⁹, —S(O) R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)— NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkyl carbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl. For some compounds according to Formula Ia, any two J groups bound to the same carbon or hetero atom may be taken together to form an oxo group.

In some embodiments, variable J in Formula Ia is halogen, amino, alkyl, haloalkyl, alkylaminyl, cycloalkyl or heterocyclyl. Alternatively, for certain Formula Ia compounds, any two J groups when bound to the same carbon or hetero atom may be taken together to form oxo group.

Further MNK-specific inhibitor compounds are compounds according to Formula IIa, illustrated below, where variable Y is —N(R⁵)— and subscript “n” is 1.

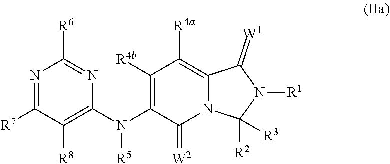

According to one embodiment, variable Y in Formula I is —O—, —S—, —C(O)—, sulfoxide, sulfone, —CHR⁹— or —CH₂—, subscript “n” is 1 and the compounds conform to Formula Hb. When “Y” is —CHR⁹— in Formula Hb, substituent R⁹ is hydrogen, lower alkyl or hydroxy.

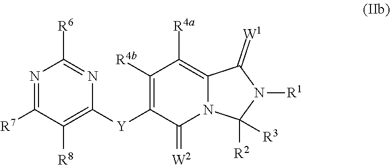

In more MNK-specific inhibitor compound embodiments, variable “Y” in Formula I is —N(R⁵)—, subscript “n” is 2 or 3 and the compounds conform to Formula Ma or Formula IVa, respectively:

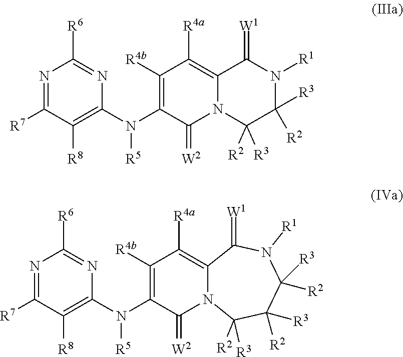

Alternatively, in certain embodiments, variable “Y” in Formula I is —O—, —S—, —C(O)—, sulfoxide, sulfone, —CHR⁹— or —CH₂—, “n” is 2 or 3 and the compounds conform to Formula Mb and Formula IVb, respectively: When “Y” is —CHR⁹— in Formula Mb or Formula IVb, substituent R⁹ is either hydrogen, lower alkyl or hydroxy.

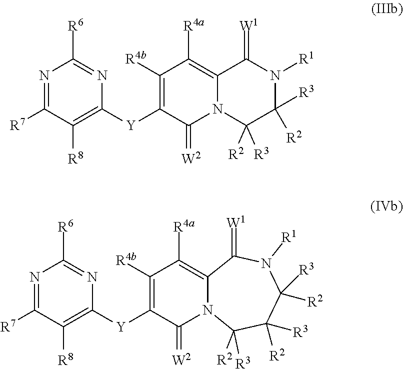

For MNK-specific inhibitor compounds according to Formulae IIa, IIb, IIIa, IIIb, IVa and IVb, variables W¹ and W² are both oxo. In certain embodiments for compounds according to Formulae IIa, IIb, IIIa, IIIb, IVa and IVb, W¹ is oxo and W² is thione group. According to one embodiment, Formulae IIa, IIb, IIIa, IIIb, IVa and IVb compounds comprise an oxo at W¹ and a ═N—OR′ group at W². Also encompassed within the scope of the present MNK-specific inhibitor compounds are Formulae IIa, IIb, IIIa, IIIb, IVa and IVb compounds having a thione group at W¹ and an oxo group at W².

For Formulae IIa, IIb, IIIa, IIIb, IVa and IVb compounds, each of substituents R² and R³ can be the same in which case the carbon atom which R² and R³ are attached is not a chiral carbon. In certain embodiments, however, substituents R² and R³ are different. Thus, the carbon atom to which R² and R³ are attached is chiral and the resulting compound will have stereoisomers.

In certain MNK-specific inhibitor compound embodiments, each R² and R³ in Formulae IIa, IIb, IIIa, IIIb, IVa and IVb is hydrogen. Alternatively, one of R² or R³ groups in Formulae IIa, IIb, IIIa, IIIb, IVa and IVb is hydrogen and the other group is alkyl optionally substituted with 1, 2 or 3 J groups. For certain compounds according to Formulae IIa, IIb, IIIa, IIIb, IVa and IVb, R² and R³ are both alkyl groups that are optionally substituted with 1, 2 or 3 J groups.

For some compounds in accordance with Formula IIa or Formula IIb, R² is alkyl and R³ is alkyl substituted with 1, 2 or 3 J groups. Exemplary of this category of Formula IIa and Formula IIb compounds are the following: compounds with substituent R² as alkyl and R³ is haloalkyl; compounds with substituent compounds with substituent R² as alkyl and R³ is cycloalkyl optionally substituted with 1, 2 or 3 J groups; compounds with substituent R² as alkyl and R³ is cyclopentyl optionally substituted with 1, 2 or 3 J groups; compounds with substituent R² as alkyl and R³ is aryl optionally substituted with 1, 2 or 3 J groups; compounds with substituent R² as alkyl and R³ is phenyl optionally substituted with 1, 2 or 3 J groups; compounds with substituent R² as alkyl and R³ is cycloalkylalkylene optionally substituted with 1, 2 or 3 J groups; compounds with substituent R² as alkyl and R³ is aralkylene optionally substituted with 1, 2 or 3 J groups; compounds with substituent R² as alkyl and R³ is benzyl optionally substituted with 1, 2 or 3 J groups; compounds with substituent R² as alkyl and R³ is heterocyclyl optionally substituted with 1, 2 or 3 J groups; compounds with substituent R² as alkyl and R³ is heteroaryl optionally substituted with 1, 2 or 3 J groups; compounds with substituent R² as alkyl and R³ is thiophenyl, thiazolyl or pyridinyl; compounds with substituent R² as alkyl and R³ is heterocyclylalkylene substituted or substituted with 1, 2 or 3 J groups; or compounds with substituent R² as alkyl and R³ is heteroarylalkylene optionally substituted with 1, 2 or 3 J groups.

In some embodiments, for compounds according to Formulae IIa, IIb, IIIa, IIIb, IVa and IVb, each R² and R³ are independently hydrogen, alkyl, cycloalkyl, cycloalkylalkylene, heterocyclyl or heterocyclylalkylene, and any such alkyl, cycloalkyl, cycloalkylalkylene, heterocyclyl or heterocyclylalkylene can optionally be substituted with 1, 2 or 3 J groups, independently selected from the group consisting of halogen, amino, alkylaminyl and alkyl.

For certain Formulae Ma, IIIb, IVa and IVb compounds, R² and R³ together with the carbon atom to which they are attached form a cycloalkyl or heterocyclyl ring.

Also contemplated are Formula I compounds where Y is —N(R⁵)—, subscript “n” is 1 and R² and R³ together with the carbon atom to which they are attached form a cycloalkyl or heterocyclyl ring “A.” Such compounds conform to Formula Va and the cycloalkyl or heterocyclyl ring “A” may optionally be substituted with 1, 2 or 3 J groups.

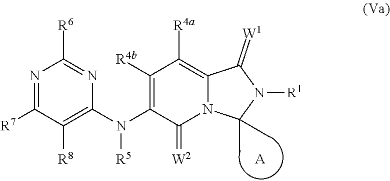

Alternatively, in some embodiments Y in Formula I is —O—, —S—, —C(O)—, sulfoxide, sulfone, —CHR⁹— or —CH₂—, “n” is 1 and R² and R³ together with the carbon atom to which they are attached form a cycloalkyl or heterocyclyl ring A. Such compounds conform to Formula Vb and the cycloalkyl or heterocyclyl ring “A” may optionally be substituted with 1, 2 or 3 J groups. When “Y” is —CHR⁹— in Formula Vb, substituent R⁹ is either hydrogen, lower alkyl or hydroxy.

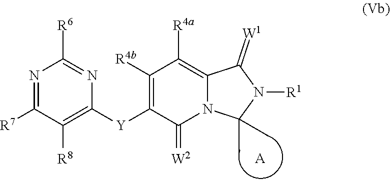

For Formula Va and Formula Vb compounds, W¹ and W² are both oxo and ring A is a cycloalkyl optionally substituted with 1, 2 or 3 J groups. Also contemplated are Formula Va and Formula Vb compounds for which ring A is a fused cycloalkyl optionally substituted with 1, 2 or 3 J groups; ring A is a cycloalkyl optionally substituted with 1, 2 or 3 J groups; ring A is a cyclobutyl, cyclopentyl or cyclohexyl optionally substituted with 1, 2 or 3 J groups, for example, J groups selected from the group consisting of halogen, amino, alkylaminyl and alkyl.

For some embodiments, ring A of a Formula Va or a Formula Vb is a heterocyclyl optionally substituted with 1, 2 or 3 J groups. Exemplary of such heterocyclyl groups are pyrrolidinyl, piperidinyl, tetrahydropyranyl, thietanyl or azetidinyl. In one embodiment, each of the above exemplified heterocyclyl may optionally be substituted with 1, 2 or 3 J groups. For certain Formula Va or a Formula Vb compounds ring A is a cycloalkyl substituted with at least 2J groups attached to the same carbon atom of the cycloalkyl, and the two J groups attached to the same carbon taken together form oxo group. In another embodiment, ring A of a Formula Va or a Formula Vb is a heterocyclyl substituted with at least 2J groups that are attached to the same hetero atom and wherein such 2 J groups taken together to form oxo. For some Formula Va or a Formula Vb compounds the cycloalkyl or heterocyclyl ring A is substituted with J groups selected from the group consisting of halogen, cyano, hydroxy, trifluoromethyl, N-methyl amino, methyl, difluoroethylene, and methylenenitrile.

The present disclosure also provides compounds in accordance with Formula VI or its stereoisomers, tautomers or pharmaceutically acceptable salts. Formula VI is a sub-genus of Formula I in which Y is —N(R⁵)— and substituent groups R⁵ and R⁸ together with the atoms to which they are attached form a heterocycle ring B which may optionally be substituted with 1, 2 or 3 J groups.

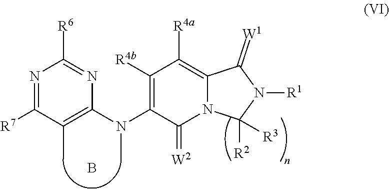

Also encompassed within the scope of the present MNK-specific inhibitor compounds are Formula I compounds in which variable “Y” is —N(R⁵)—, and substituent groups R⁷ and R⁸ together with the atoms to which they are attached form a fused ring C. Such compounds or the stereoisomer, tautomer or pharmaceutically acceptable salt conform to Formula VIIa. For Formula VIIa compounds, ring C may optionally be substituted with 1, 2 or 3 J groups.

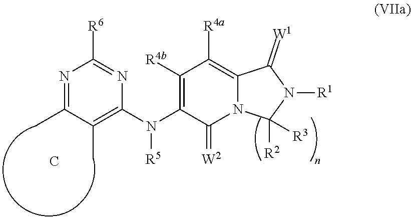

According to one embodiment, variable “Y” in Formula I is —O—, —S—, —C(O)—, sulfoxide, sulfone, —CHR⁹— or —CH₂—, and substituent groups R⁷ and R⁸ together with the atoms to which they are attached form a fused ring C. Such compounds and their stereoisomers, tautomers or pharmaceutically acceptable salts conform to Formula VIIb. For Formula VIIb compounds where “Y” is —CHR⁹—, substituent R⁹ can be hydrogen, lower alkyl or hydroxy.

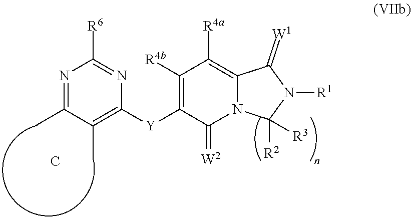

For Formula VIIb compounds, fused ring C may optionally be substituted with 1, 2 or 3 J groups. In one MNK-specific inhibitor embodiment, W¹ and W² are both oxo for Formula VI, Formula VIIa and Formula VIIb compounds.

MNK-specific inhibitor compounds of this disclosure are further directed to Formulae I, Ia, IIa, IIb, IIIa, IIIb, IVa, IVb, Va, Vb, VI, VIIa and VIIb compounds where R¹ is hydrogen or a lower alkyl group selected from methyl, ethyl, propyl, butyl, iso-propyl, sec-butyl, or tert-butyl, for example, compounds with R¹ as methyl.

For certain Formulae I, Ia, IIa, IIb, IIIa, IIIb, IVa, IVb, Va, Vb, VI, Vila and VIIb compounds, R^{4a} is selected from the group consisting of hydrogen, halogen, alkyl, alkoxy, thioalkyl, alkenyl, and cycloalkyl while substituent R^{4b} is hydrogen or halogen. R⁵ in Formulae I, Ia, IIa, IIb, IIIa, IIIb, IVa, IVb, Va, Vb, VI, Vila and VIIb is hydrogen or lower alkyl, while substituents R⁶, R⁷ and R⁸ are hydrogen.

In certain embodiments of this disclosure, R⁶ and R⁷ in Formula VI are both hydrogen, while for certain Formula Vila and Formula VIIb compounds R⁶ is hydrogen.

MNK-specific inhibitor compounds of this disclosure are further directed to Formulae I, Ia, IIa, IIb, IIIa, IIIb, IVa, IVb, Va, and Vb compounds where substituent groups R⁶ and R⁸ are both hydrogen, and R₇ is selected from the group consisting of hydroxy, halogen, cyano, alkyl, alkenyl, alkynyl, alkoxy, cycloalkyl cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, and heterocyclyl. For these compounds, any alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups. In certain embodiments, R₇ is selected from the group consisting of alkyl, cycloalkyl, cycloalkylalkylene, cycloalkylalkenylene, amino, alkylaminyl, alklycarbonylaminyl, cycloalkylcarbonylaminyl, heterocyclylaminyl, heteroaryl, heterocyclyl and cycloalkylaminyl. For such compounds any alkyl, alkenyl, cycloalkyl, cycloalkylalkylene, cycloalkylalkenylene, amino, alkylaminyl, alklycarbonylaminyl, cycloalkylcarbonylaminyl, heterocyclylaminyl, heteroaryl, heterocyclyl or cycloalkylaminyl may optionally be substituted with 1, 2 or 3 J groups. Thus, certain embodiments provide Formulae I, Ia, IIa, IIb, IIIa, IIIb, IVa, IVb, Va, and Vb compounds where substituent groups R⁶ and R⁸ are both hydrogen, and R₇ is amino; substituent groups R⁶ and R⁸ are both hydrogen, and R₇ is alkylaminyl; substituent groups R⁶ and R⁸ are both hydrogen, and R₇ is —NHCH₃; substituent groups R⁶ and R⁸ are both hydrogen, and R₇ is cycloalkyl, for example cyclopropyl; substituent groups R⁶ and R⁸ are both hydrogen, and R₇ is cycloalkylaminyl substituted with 1 to 3 J groups, for instance halogens.

In one embodiment, for compounds in accordance with Formulae I, Ia, IIa, IIb, IIIa, IIIb, IVa, IVb, Va, and Vb, substituent groups R⁶ and R⁸ are both hydrogen, and R₇ is selected from the group consisting of —NHCH(CF₃)cyclopropyl, cycloalkylcarbonylaminyl, — NHC(O)cyclopropyl, cycloalkylalkenylene, and —CH═CHcyclopropyl.

For any compound in accordance with Formulae I, Ia, IIa, IIb, IIIa, IIIb, IVa, IVb, Va, Vb, VI, Vila, and VIIb, J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl and R⁹ is hydrogen, lower alkyl or —OH. Additionally, when two J groups bound to the same carbon or hetero atom they may be taken together to form oxo.

For certain compounds according to Formulae I, Ia, IIa, IIb, IIIa, IIIb, IVa, IVb, Va, Vb, VI, Vila, and VIIb, J is halogen, hydroxy, alkyl, alkenyl, alkynyl or cyanoalkylene. Illustrative alkyl or alkylene chains are those having C₁-C₁₀ carbon atoms, C₁-C₈ carbon atoms, C₁-C₆ carbon atoms, C₁-C₄ carbon atoms, C₁-C₃ carbon atoms as well as ethyl and methyl groups. Alternatively, when J is alkenyl, or alkynyl, the carbon chain has at least one double or triple bond respectively and C₂-C₁₀ carbon atoms, C₂-C₈ carbon atoms, C₂-C₆ carbon atoms, C₂-C₄ carbon atoms, or C₂-C₃ carbon atoms.

A MNK-specific inhibitor compound of Formula (I), as well as Formulae Ia, IIa, lib, IIIa, Mb, IVa, IVb, Va, Vb, VI, Vila and VIIb, may be isotopically-labelled by having one or more atoms replaced by an atom having a different atomic mass or mass number. Examples of isotopes that can be incorporated into the compounds of structure (I) include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I respectively. These radiolabelled compounds may be useful to help determine or measure the effectiveness of the compounds, by characterizing, for example, the site or mode of action, or binding affinity to pharmacologically important site of action. Certain isotopically-labelled compounds of Formula (I), for example, those incorporating a radioactive isotope, are useful in drug or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), as well as Formulae Ia, IIa, IIb, IIIa, nib, IVa, IVb, Va, Vb, VI, Vila and VIIb, can generally be prepared by conventional techniques known to those skilled in the art or by processes analogous to those described in the Preparations and Examples as set out in U.S. patent application Ser. No. 14/748,990 filed Jun. 24, 2015 and entitled “MNK Inhibitors and Methods Related Thereto,” which compounds and synthetic methods are incorporated herein in their entirety, using an appropriate isotopically-labeled reagent in place of the non-labeled reagent previously employed.

Embodiments of this disclosure are also meant to encompass the in vivo metabolic products of the MNK-specific inhibitor compounds of Formulae I, Ia, IIa, IIb, IIIa, IIIb, IVa, IVb, Va, Vb, VI, Vila and VIIb. Such products may result from, for example, the oxidation, reduction, hydrolysis, amidation, esterification, and the like of the administered compound, primarily due to enzymatic processes. Accordingly, the instant disclosure includes compounds produced by a process comprising administering a MNK-specific inhibitor compound of this disclosure to a mammal for a period of time sufficient to yield a metabolic product thereof. Such products are typically identified by administering a radiolabelled MNK-specific inhibitor as described herein in a detectable dose to an animal, such as rat, mouse, guinea pig, monkey, or human, allowing sufficient time for metabolism to occur, and isolating conversion products from the urine, blood or other biological samples.

In some embodiments, a MNK-specific inhibitor compound of any one of compounds according to Formulae I, Ia, IIa, IIb, IIIa, IIIb, IVa, IVb, Va, Vb, VI, VIIa and VIIb are in the form of a pharmaceutically acceptable salt, which includes both acid and base addition salts.

To this end, a “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, or the like.

Similarly, a “pharmaceutically acceptable base addition salt” refers to those salts which retain the biological effectiveness and properties of the free acids, which are not biologically or otherwise undesirable. These salts are prepared by addition of an inorganic base or an organic base to the free acid. Salts derived from inorganic bases include the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, di cyclohexylamine, choline and caffeine.

Often crystallizations produce a solvate of a MNK-specific inhibitor compound of this disclosure. As used herein, the term “solvate” refers to an aggregate that comprises one or more molecules of a compound of the present disclosure with one or more molecules of solvent. A solvent may be water, in which case the solvate may be a hydrate. Alternatively, a solvent may be an organic solvent. Thus, the MNK-specific inhibitor compounds of the present disclosure may exist as a hydrate, including a monohydrate, dihydrate, hemihydrate, sesquihydrate, trihydrate, tetrahydrate or the like, as well as the corresponding solvated forms. The MNK-specific inhibitor compounds of this disclosure may be true solvates, while in other cases, the compounds may merely retain adventitious water or be a mixture of water plus some adventitious solvent.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposeable mirror images of one another.

MNK-specific inhibitor compounds of this disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The term “tautomer” refers to a proton shift from one atom of a molecule to another atom of the same molecule. For example, when W¹ is oxo and R¹ is H, the present disclosure provides tautomers of a Formula I compound as illustrated below:

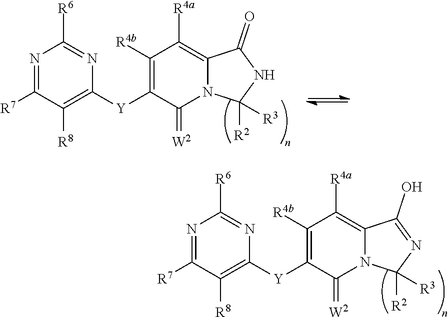

Similar tautomers exists for Formulae I, Ia, IIa, Hb, IIIa, IIIb, IVa, IVb, Va, Vb, VI, VIIa and VIIb compounds. The compounds are synthesized using conventional synthetic methods, and more specifically using the general methods and specific synthetic protocols of the Examples found in U.S. patent application Ser. No. 14/748,990 filed Jun. 24, 2015 and entitled “MNK Inhibitors and Methods Related Thereto,” which compounds and synthetic methods are incorporated herein in their entirety.

Representative MNK-specific inhibitor compounds of this disclosure are set forth in Table B and in U.S. Patent Application Publication No. US 2015/0376181, which compounds are incorporated herein by reference in their entirety. Similarly, incorporated herein by reference in their entirety are compounds and methods of making the same from U.S. Pat. No. 10,112,955, claiming priority to U.S. Provisional Patent Application No. 62/247,953 (entitled “Isoindoline, Azaisoindoline, Dihydroindenone and Dihydroazaindenone Inhibitors of MNK1 and MNK2”) and U.S. application Ser. No. 15/337,237, claiming priority to U.S. Provisional Patent Application No. 62/247,966 (entitled “Pyrrolo-, Pyrazolo-, Imidazo-Pyrimidine and Pyridine Compounds that Inhibit MNK1 and MNK2”). Such compounds are provided for purpose of illustration and not limitation.

TABLE B
Exemplary MNK-Specific Inhibitors

| Cmpd. No. | Structure |
|---|---|
| 1 | 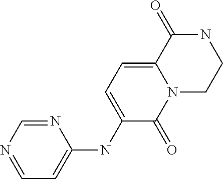 [SMILES: O=C1NCCn2c1ccc(Nc1ccncn1)c2=O] |
| 2 | 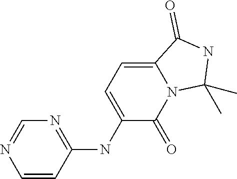 [SMILES: CC1(C)NC(=O)c2ccc(Nc3ccncn3)c(=O)n21] |
| 3 | 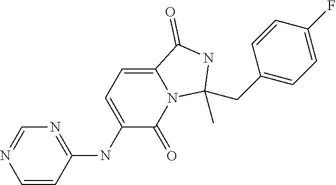 [SMILES: CC1(Cc2ccc(F)cc2)NC(=O)c2ccc(Nc3ccncn3)c(=O)n21] |
| 4 | 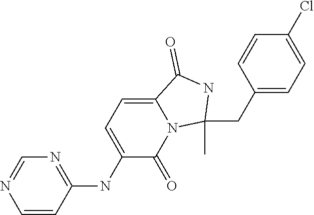 [SMILES: CC1(Cc2ccc(Cl)cc2)NC(=O)c2ccc(Nc3ccncn3)c(=O)n21] |
| 5 | 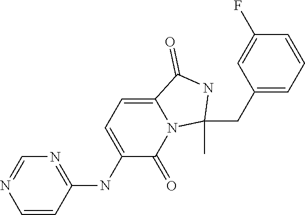 [SMILES: CC1(Cc2cccc(F)c2)NC(=O)c2ccc(Nc3ccncn3)c(=O)n21] |
| 6 | 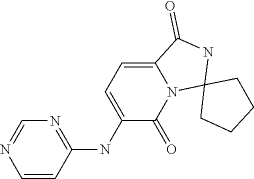 [SMILES: O=C1NC2(CCCC2)n2c1ccc(Nc1ccncn1)c2=O] |
| 7 | 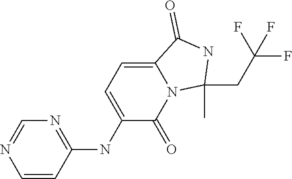 [SMILES: CC1(CC(F)(F)F)NC(=O)c2ccc(Nc3ccncn3)c(=O)n21] |
| 8 | 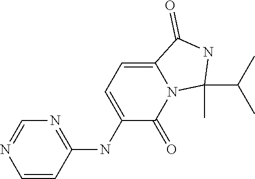 [SMILES: CC(C)C1(C)NC(=O)c2ccc(Nc3ccncn3)c(=O)n21] |
| 9 | 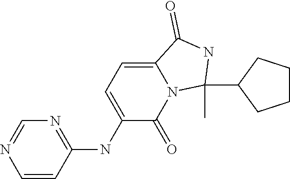 [SMILES: CC1(C2CCCC2)NC(=O)c2ccc(Nc3ccncn3)c(=O)n21] |
| 10 | 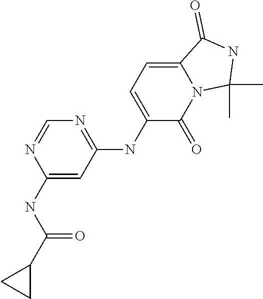 [SMILES: CC1(C)NC(=O)c2ccc(Nc3cc(NC(=O)C4CC4)ncn3)c(=O)n21] |
| 11 | 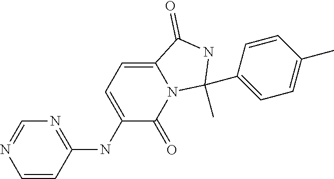 [SMILES: Cc1ccc(C2(C)NC(=O)c3ccc(Nc4ccncn4)c(=O)n32)cc1] |
| 12 | 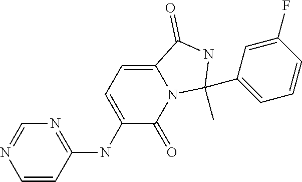 [SMILES: CC1(c2cccc(F)c2)NC(=O)c2ccc(Nc3ccncn3)c(=O)n21] |
| 13 | 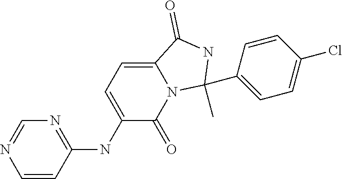 [SMILES: CC1(c2ccc(Cl)cc2)NC(=O)c2ccc(Nc3ccncn3)c(=O)n21] |
| 14 | 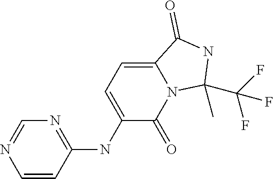 [SMILES: CC1(C(F)(F)F)NC(=O)c2ccc(Nc3ccncn3)c(=O)n21] |
| 15 | 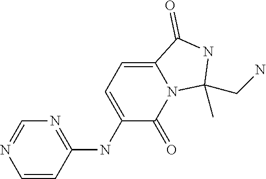 [SMILES: CC1(CN)NC(=O)c2ccc(Nc3ccncn3)c(=O)n21] |
| 16 | 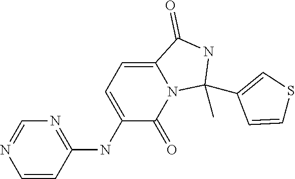 [SMILES: CC1(c2ccsc2)NC(=O)c2ccc(Nc3ccncn3)c(=O)n21] |
| 17 | 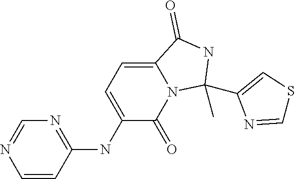 [SMILES: CC1(c2cscn2)NC(=O)c2ccc(Nc3ccncn3)c(=O)n21] |
| 18 | 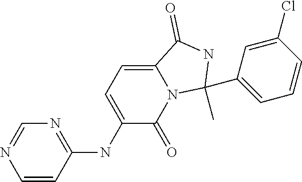 [SMILES: CC1(c2cccc(Cl)c2)NC(=O)c2ccc(Nc3ccncn3)c(=O)n21] |
| 19 | 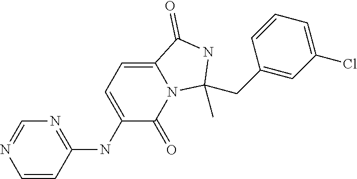 [SMILES: CC1(Cc2cccc(Cl)c2)NC(=O)c2ccc(Nc3ccncn3)c(=O)n21] |
| 20 | 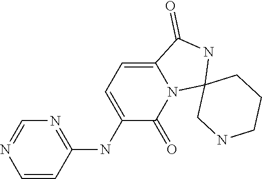 [SMILES: O=C1NC2(CCCNC2)n2c1ccc(Nc1ccncn1)c2=O] |
| 21 | 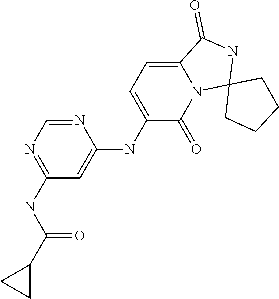 [SMILES: O=C1NC2(CCCC2)n2c1ccc(Nc1cc(NC(=O)C3CC3)ncn1)c2=O] |
| 22 | 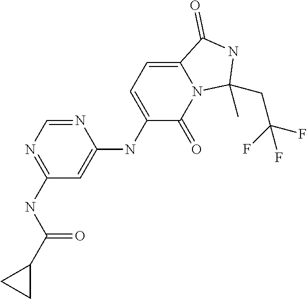 [SMILES: CC1(CC(F)(F)F)NC(=O)c2ccc(Nc3cc(NC(=O)C4CC4)ncn3)c(=O)n21] |
| 23 | 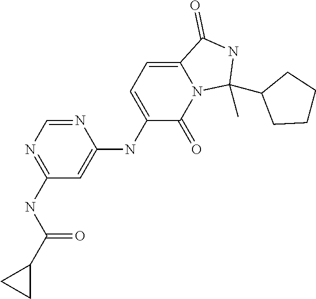 [SMILES: CC1(C2CCCC2)NC(=O)c2ccc(Nc3cc(NC(=O)C4CC4)ncn3)c(=O)n21] |
| 24 | 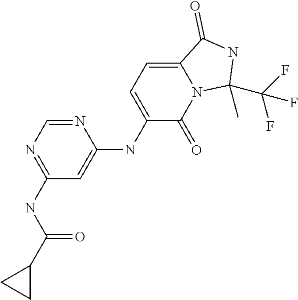 [SMILES: CC1(C(F)(F)F)NC(=O)c2ccc(Nc3cc(NC(=O)C4CC4)ncn3)c(=O)n21] |
| 25 | 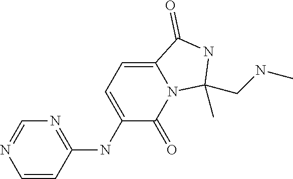 [SMILES: CNCC1(C)NC(=O)c2ccc(Nc3ccncn3)c(=O)n21] |
| 26 | 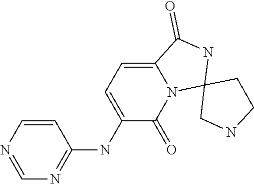 [SMILES: O=C1NC2(CCNC2)n2c1ccc(Nc1ccncn1)c2=O] |
| 27 | 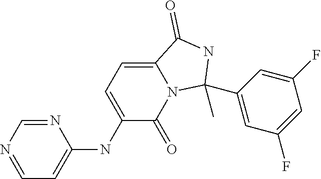 [SMILES: CC1(c2cc(F)cc(F)c2)NC(=O)c2ccc(Nc3ccncn3)c(=O)n21] |
| 28 | 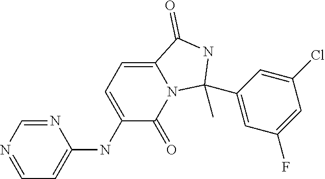 [SMILES: CC1(c2cc(F)cc(Cl)c2)NC(=O)c2ccc(Nc3ccncn3)c(=O)n21] |
| 29 | 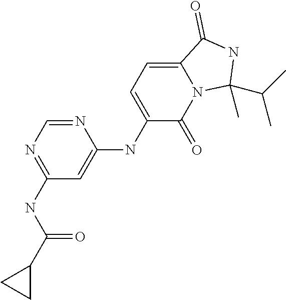 [SMILES: CC(C)C1(C)NC(=O)c2ccc(Nc3cc(NC(=O)C4CC4)ncn3)c(=O)n21] |
| 30 | 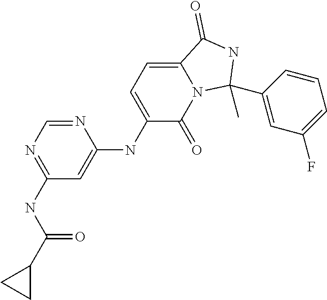 [SMILES: CC1(c2cccc(F)c2)NC(=O)c2ccc(Nc3cc(NC(=O)C4CC4)ncn3)c(=O)n21] |
| 31 | 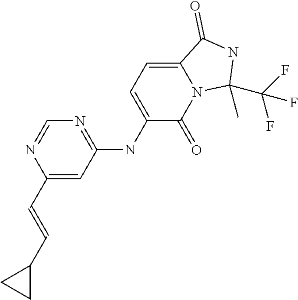 [SMILES: CC1(C(F)(F)F)NC(=O)c2ccc(Nc3cc(/C=C/C4CC4)ncn3)c(=O)n21] |
| 32 | 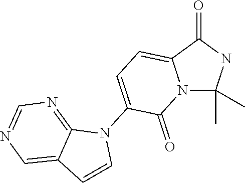 [SMILES: CC1(C)NC(=O)c2ccc(-n3ccc4cncnc43)c(=O)n21] |
| 33 | 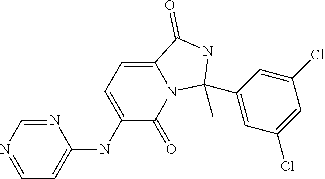 [SMILES: CC1(c2cc(Cl)cc(Cl)c2)NC(=O)c2ccc(Nc3ccncn3)c(=O)n21] |
| 34 | 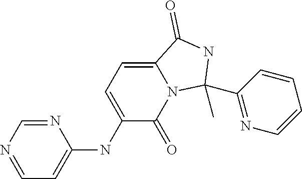 [SMILES: CC1(c2ccccn2)NC(=O)c2ccc(Nc3ccncn3)c(=O)n21] |
| 35 | 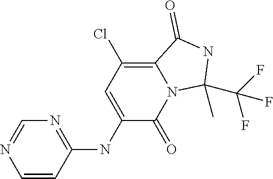 [SMILES: CC1(C(F)(F)F)NC(=O)c2c(Cl)cc(Nc3ccncn3)c(=O)n21] |
| 36 | 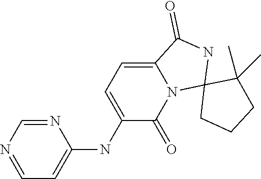 [SMILES: CC1(C)CCCC12NC(=O)c1ccc(Nc3ccncn3)c(=O)n12] |
| 37 | 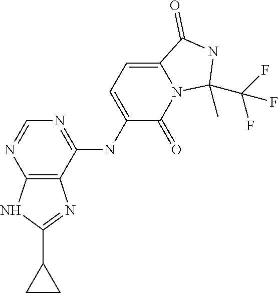 [SMILES: CC1(C(F)(F)F)NC(=O)c2ccc(Nc3ncnc4[nH]c(C5CC5)nc34)c(=O)n21] |
| 38 | 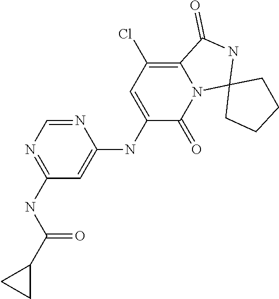 [SMILES: O=C1NC2(CCCC2)n2c1c(Cl)cc(Nc1cc(NC(=O)C3CC3)ncn1)c2=O] |
| 39 | 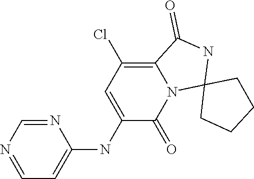 [SMILES: O=C1NC2(CCCC2)n2c1c(Cl)cc(Nc1ccncn1)c2=O] |
| 40 | 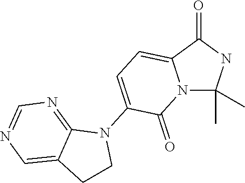 [SMILES: CC1(C)NC(=O)c2ccc(N3CCc4cncnc43)c(=O)n21] |
| 41 | 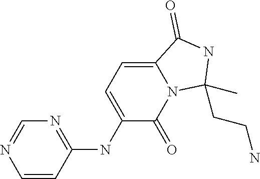 [SMILES: CC1(CCN)NC(=O)c2ccc(Nc3ccncn3)c(=O)n21] |
| 42 | 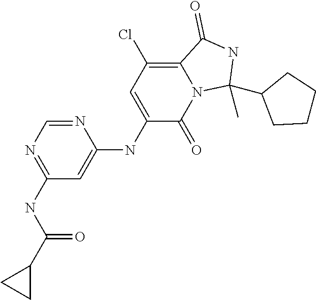 [SMILES: CC1(C2CCCC2)NC(=O)c2c(Cl)cc(Nc3cc(NC(=O)C4CC4)ncn3)c(=O)n21] |
| 43 | 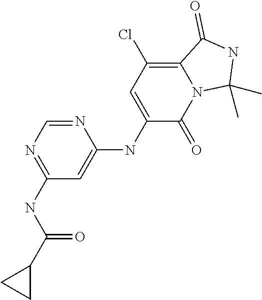 [SMILES: CC1(C)NC(=O)c2c(Cl)cc(Nc3cc(NC(=O)C4CC4)ncn3)c(=O)n21] |
| 44 | 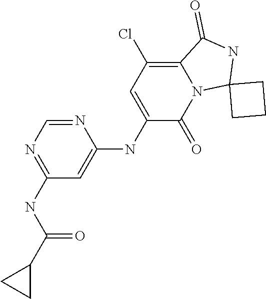 [SMILES: O=C1NC2(CCC2)n2c1c(Cl)cc(Nc1cc(NC(=O)C3CC3)ncn1)c2=O] |
| 45 | 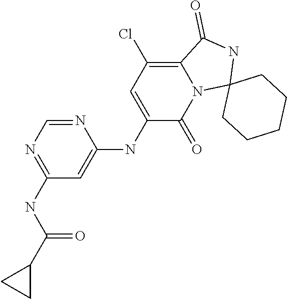 [SMILES: O=C1NC2(CCCCC2)n2c1c(Cl)cc(Nc1cc(NC(=O)C3CC3)ncn1)c2=O] |
| 46 | 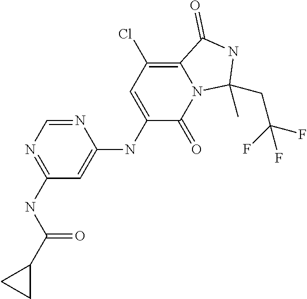 [SMILES: CC1(CC(F)(F)F)NC(=O)c2c(Cl)cc(Nc3cc(NC(=O)C4CC4)ncn3)c(=O)n21] |
| 47 | 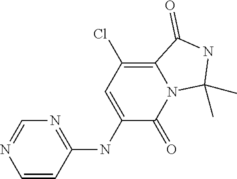 [SMILES: CC1(C)NC(=O)c2c(Cl)cc(Nc3ccncn3)c(=O)n21] |
| 48 | 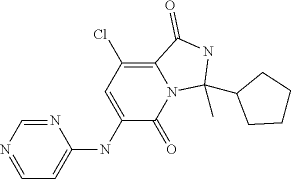 [SMILES: CC1(C2CCCC2)NC(=O)c2c(Cl)cc(Nc3ccncn3)c(=O)n21] |
| 49 | 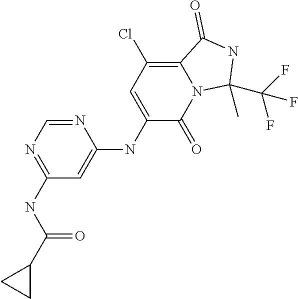 [SMILES: CC1(C(F)(F)F)NC(=O)c2c(Cl)cc(Nc3cc(NC(=O)C4CC4)ncn3)c(=O)n21] |
| 50 | 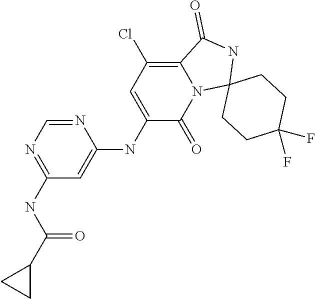 [SMILES: O=C1NC2(CCC(F)(F)CC2)n2c1c(Cl)cc(Nc1cc(NC(=O)C3CC3)ncn1)c2=O] |
| 51 | 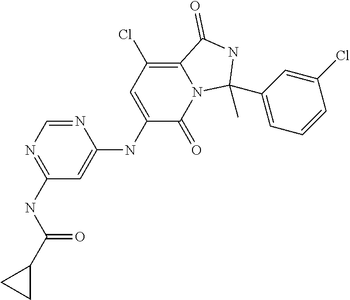 [SMILES: CC1(c2cccc(Cl)c2)NC(=O)c2c(Cl)cc(Nc3cc(NC(=O)C4CC4)ncn3)c(=O)n21] |
| 52 | 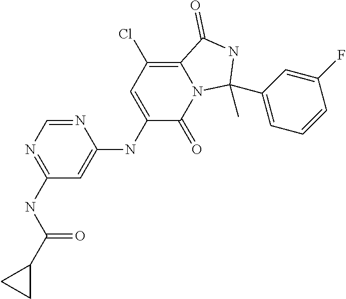 [SMILES: CC1(c2cccc(F)c2)NC(=O)c2c(Cl)cc(Nc3cc(NC(=O)C4CC4)ncn3)c(=O)n21] |
| 53 | 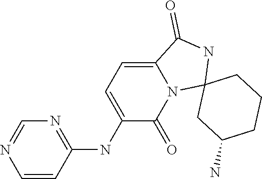 [SMILES: N[C@H]1CCCC2(C1)NC(=O)c1ccc(Nc3ccncn3)c(=O)n12] |
| 54 | 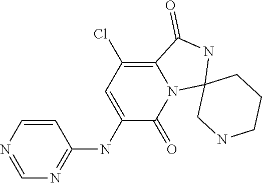 [SMILES: O=C1NC2(CCCNC2)n2c1c(Cl)cc(Nc1ccncn1)c2=O] |
| 55 | 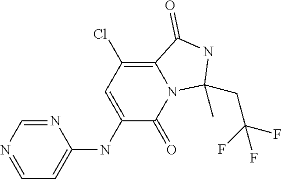 [SMILES: CC1(CC(F)(F)F)NC(=O)c2c(Cl)cc(Nc3ccncn3)c(=O)n21] |
| 56 | 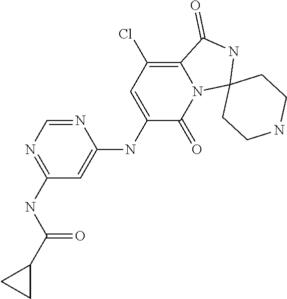 [SMILES: O=C1NC2(CCNCC2)n2c1c(Cl)cc(Nc1cc(NC(=O)C3CC3)ncn1)c2=O] |
| 57 | 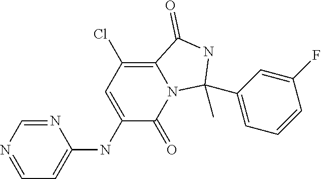 [SMILES: CC1(c2cccc(F)c2)NC(=O)c2c(Cl)cc(Nc3ccncn3)c(=O)n21] |
| 58 | 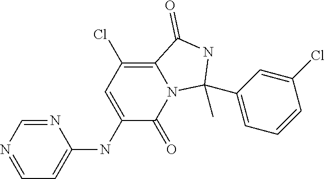 [SMILES: CC1(c2cccc(Cl)c2)NC(=O)c2c(Cl)cc(Nc3ccncn3)c(=O)n21] |
| 59 | 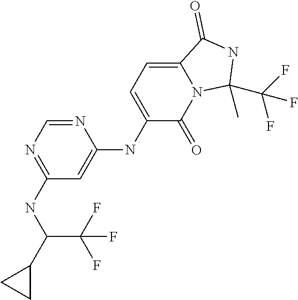 [SMILES: CC1(C(F)(F)F)NC(=O)c2ccc(Nc3cc(NC(C4CC4)C(F)(F)F)ncn3)c(=O)n21] |
| 60 | 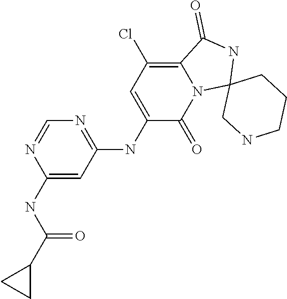 [SMILES: O=C1NC2(CCCNC2)n2c1c(Cl)cc(Nc1cc(NC(=O)C3CC3)ncn1)c2=O] |
| 61 | 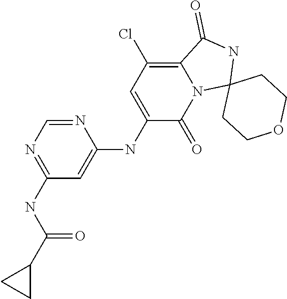 [SMILES: O=C1NC2(CCOCC2)n2c1c(Cl)cc(Nc1cc(NC(=O)C3CC3)ncn1)c2=O] |
| 62 | 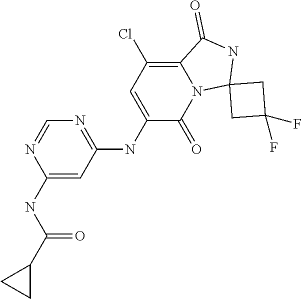 [SMILES: O=C1NC2(CC(F)(F)C2)n2c1c(Cl)cc(Nc1cc(NC(=O)C3CC3)ncn1)c2=O] |
| 63 | 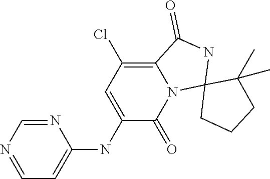 [SMILES: CC1(C)CCCC12NC(=O)c1c(Cl)cc(Nc3ccncn3)c(=O)n12] |
| 64 | 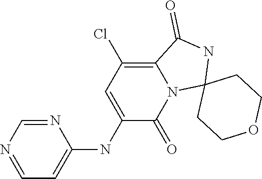 [SMILES: O=C1NC2(CCOCC2)n2c1c(Cl)cc(Nc1ccncn1)c2=O] |
| 65 | 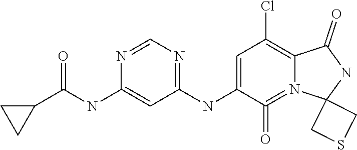 |
| 66 | 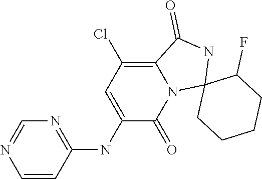 [SMILES: O=C1NC2(CCCCC2F)n2c1c(Cl)cc(Nc1ccncn1)c2=O] |
| 67 | 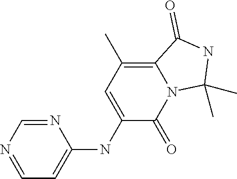 [SMILES: Cc1cc(Nc2ccncn2)c(=O)n2c1C(=O)NC2(C)C] |
| 68 | 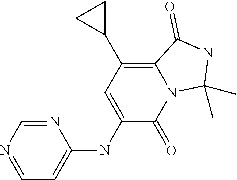 [SMILES: CC1(C)NC(=O)c2c(C3CC3)cc(Nc3ccncn3)c(=O)n21] |
| 69 | 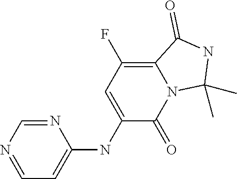 [SMILES: CC1(C)NC(=O)c2c(F)cc(Nc3ccncn3)c(=O)n21] |
| 70 | 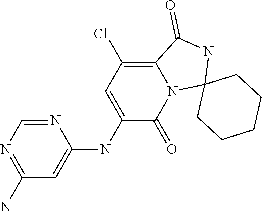 [SMILES: Nc1cc(Nc2cc(Cl)c3n(c2=O)C2(CCCCC2)NC3=O)ncn1] |
| 71 | 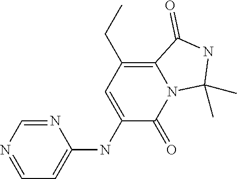 [SMILES: CCc1cc(Nc2ccncn2)c(=O)n2c1C(=O)NC2(C)C] |
| 72 | 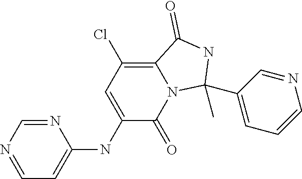 [SMILES: CC1(c2cccnc2)NC(=O)c2c(Cl)cc(Nc3ccncn3)c(=O)n21] |
| 73 | 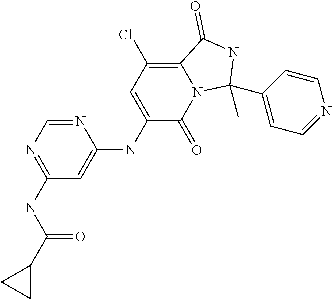 [SMILES: CC1(c2ccncc2)NC(=O)c2c(Cl)cc(Nc3cc(NC(=O)C4CC4)ncn3)c(=O)n21] |
| 74 | 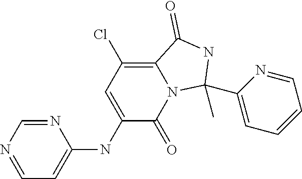 [SMILES: CC1(c2ccccn2)NC(=O)c2c(Cl)cc(Nc3ccncn3)c(=O)n21] |
| 75 | 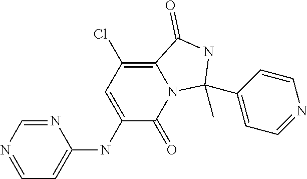 [SMILES: CC1(c2ccncc2)NC(=O)c2c(Cl)cc(Nc3ccncn3)c(=O)n21] |
| 76 | 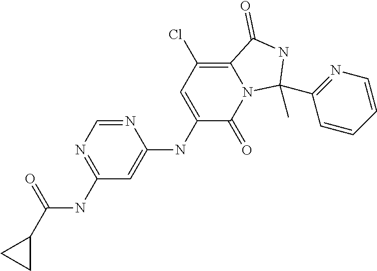 [SMILES: CC1(c2ccccn2)NC(=O)c2c(Cl)cc(Nc3cc(NC(=O)C4CC4)ncn3)c(=O)n21] |
| 77 | 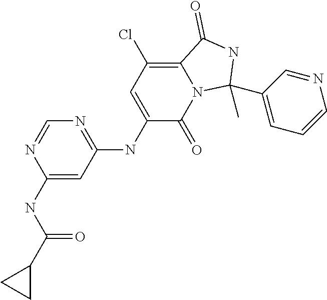 [SMILES: CC1(c2cccnc2)NC(=O)c2c(Cl)cc(Nc3cc(NC(=O)C4CC4)ncn3)c(=O)n21] |
| 78 | 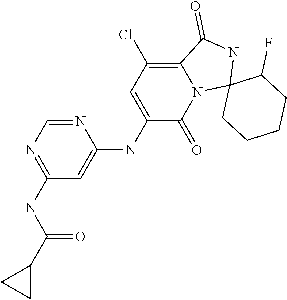 [SMILES: O=C1NC2(CCCCC2F)n2c1c(Cl)cc(Nc1cc(NC(=O)C3CC3)ncn1)c2=O] |
| 79 | 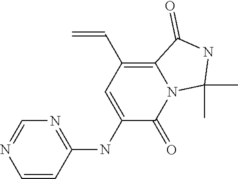 [SMILES: C=Cc1cc(Nc2ccncn2)c(=O)n2c1C(=O)NC2(C)C] |
| 80 | 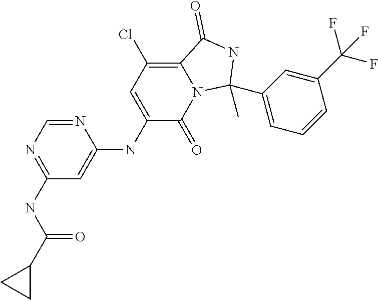 [SMILES: CC1(c2cccc(C(F)(F)F)c2)NC(=O)c2c(Cl)cc(Nc3cc(NC(=O)C4CC4)ncn3)c(=O)n21] |
| 81 | 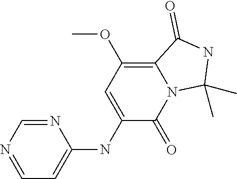 [SMILES: COc1cc(Nc2ccncn2)c(=O)n2c1C(=O)NC2(C)C] |
| 82 | 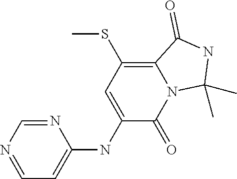 [SMILES: CSc1cc(Nc2ccncn2)c(=O)n2c1C(=O)NC2(C)C] |
| 83 | 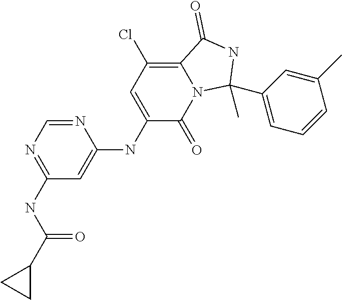 [SMILES: Cc1cccc(C2(C)NC(=O)c3c(Cl)cc(Nc4cc(NC(=O)C5CC5)ncn4)c(=O)n32)c1] |
| 84 | 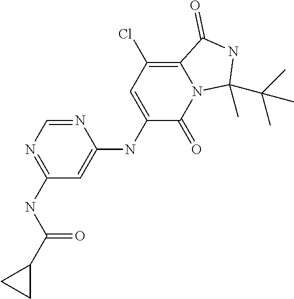 [SMILES: CC(C)(C)C1(C)NC(=O)c2c(Cl)cc(Nc3cc(NC(=O)C4CC4)ncn3)c(=O)n21] |
| 85 | 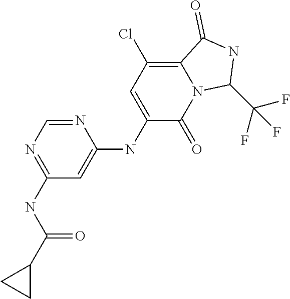 [SMILES: O=C1NC(C(F)(F)F)n2c1c(Cl)cc(Nc1cc(NC(=O)C3CC3)ncn1)c2=O] |
| 86 | 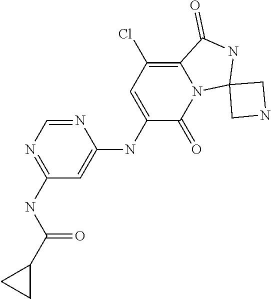 [SMILES: O=C1NC2(CNC2)n2c1c(Cl)cc(Nc1cc(NC(=O)C3CC3)ncn1)c2=O] |
| 87 | 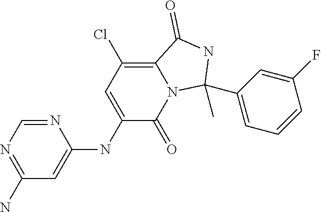 [SMILES: CC1(c2cccc(F)c2)NC(=O)c2c(Cl)cc(Nc3cc(N)ncn3)c(=O)n21] |
| 88 | 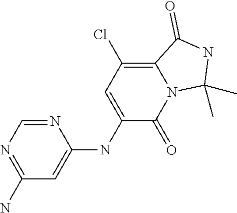 [SMILES: CC1(C)NC(=O)c2c(Cl)cc(Nc3cc(N)ncn3)c(=O)n21] |
| 89 | 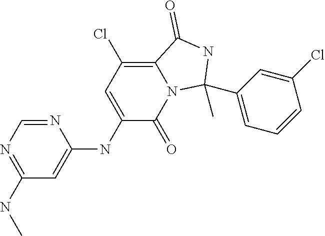 [SMILES: CNc1cc(Nc2cc(Cl)c3n(c2=O)C(C)(c2cccc(Cl)c2)NC3=O)ncn1] |
| 90 | 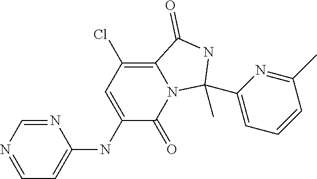 [SMILES: Cc1cccc(C2(C)NC(=O)c3c(Cl)cc(Nc4ccncn4)c(=O)n32)n1] |
| 91 | 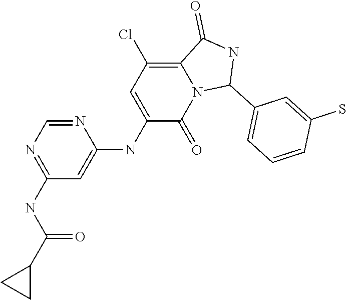 [SMILES: O=C1NC(c2cccc(S)c2)n2c1c(Cl)cc(Nc1cc(NC(=O)C3CC3)ncn1)c2=O] |
| 92 | 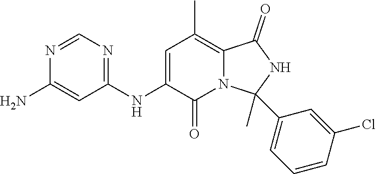 [SMILES: Cc1cc(Nc2cc(N)ncn2)c(=O)n2c1C(=O)NC2(C)c1cccc(Cl)c1] |
| 93 | 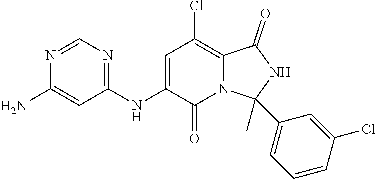 [SMILES: CC1(c2cccc(Cl)c2)NC(=O)c2c(Cl)cc(Nc3cc(N)ncn3)c(=O)n21] |
| 94 | 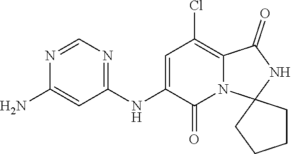 [SMILES: Nc1cc(Nc2cc(Cl)c3n(c2=O)C2(CCCC2)NC3=O)ncn1] |
| 95 | 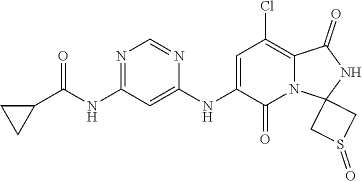 [SMILES: O=C1NC2(CS(=O)C2)n2c1c(Cl)cc(Nc1cc(NC(=O)C3CC3)ncn1)c2=O] |
| 96 | 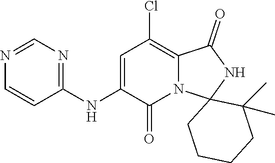 [SMILES: CC1(C)CCCCC12NC(=O)c1c(Cl)cc(Nc3ccncn3)c(=O)n12] |
| 97 | 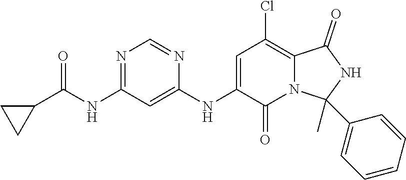 [SMILES: CC1(c2ccccc2)NC(=O)c2c(Cl)cc(Nc3cc(NC(=O)C4CC4)ncn3)c(=O)n21] |
| 98 | 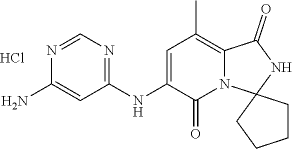 [SMILES: Cc1cc(Nc2cc(N)ncn2)c(=O)n2c1C(=O)NC21CCCC1 ; Cl] |
| 99 | 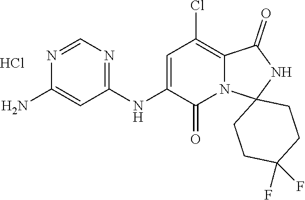 [SMILES: Cl ; Nc1cc(Nc2cc(Cl)c3n(c2=O)C2(CCC(F)(F)CC2)NC3=O)ncn1] |
| 100 | 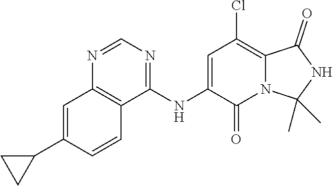 [SMILES: CC1(C)NC(=O)c2c(Cl)cc(Nc3ncnc4cc(C5CC5)ccc34)c(=O)n21] |
| 101 | 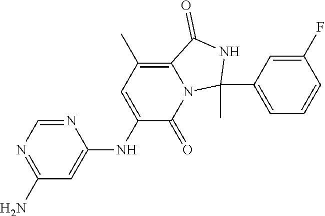 [SMILES: Cc1cc(Nc2cc(N)ncn2)c(=O)n2c1C(=O)NC2(C)c1cccc(F)c1] |
| 102 | 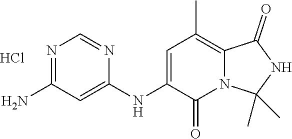 [SMILES: Cc1cc(Nc2cc(N)ncn2)c(=O)n2c1C(=O)NC2(C)C ; Cl] |
| 103 | 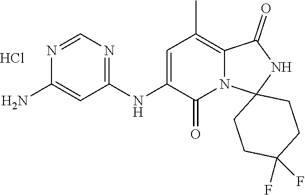 [SMILES: Cc1cc(Nc2cc(N)ncn2)c(=O)n2c1C(=O)NC21CCC(F)(F)CC1 ; Cl] |
| 104 | 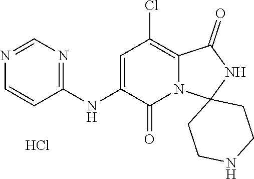 [SMILES: O=C1NC2(CCNCC2)n2c1c(Cl)cc(Nc1ccncn1)c2=O ; Cl] |
| 105 | 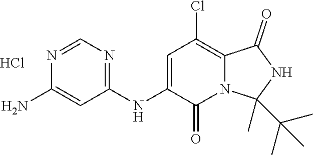 [SMILES: CC(C)(C)C1(C)NC(=O)c2c(Cl)cc(Nc3cc(N)ncn3)c(=O)n21 ; Cl] |
| 106 | 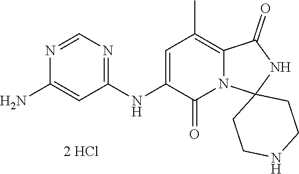 [SMILES: Cc1cc(Nc2cc(N)ncn2)c(=O)n2c1C(=O)NC21CCNCC1 ; "2 HCl"] |
| 107 | 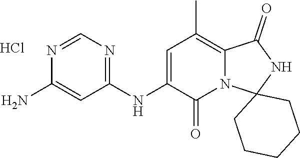 [SMILES: Cl ; Cc1cc(Nc2cc(N)ncn2)c(=O)n2c1C(=O)NC21CCCCC1] |
| 108 | 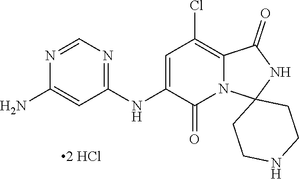 [SMILES: Nc1cc(Nc2cc(Cl)c3n(c2=O)C2(CCNCC2)NC3=O)ncn1 ; "·2 HCl"] |
| 109 | 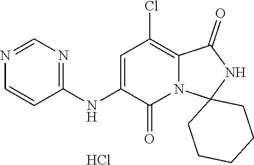 [SMILES: O=C1NC2(CCCCC2)n2c1c(Cl)cc(Nc1ccncn1)c2=O ; Cl] |
| 110 | 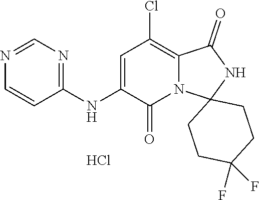 [SMILES: O=C1NC2(CCC(F)(F)CC2)n2c1c(Cl)cc(Nc1ccncn1)c2=O ; Cl] |
| 111 | 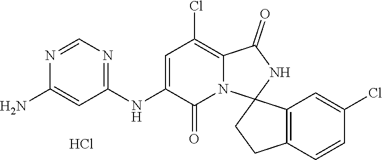 [SMILES: Nc1cc(Nc2cc(Cl)c3n(c2=O)C2(CCc4ccc(Cl)cc42)NC3=O)ncn1 ; Cl] |
| 112 | 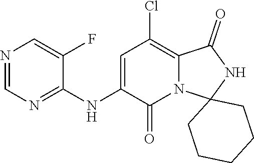 [SMILES: O=C1NC2(CCCCC2)n2c1c(Cl)cc(Nc1ncncc1F)c2=O] |
| 440 | 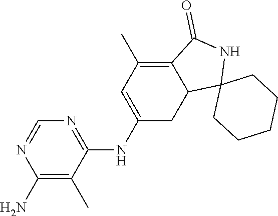 [SMILES: CC1=C2C(=O)NC3(CCCCC3)C2CC(Nc2ncnc(N)c2C)=C1] |
| 462 | 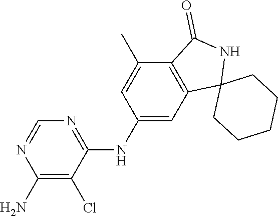 [SMILES: Cc1cc(Nc2ncnc(N)c2Cl)cc2c1C(=O)NC21CCCCC1] |
| 474 | 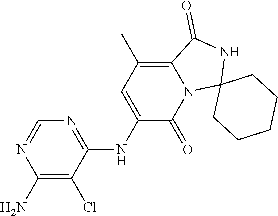 [SMILES: Cc1cc(Nc2ncnc(N)c2Cl)c(=O)n2c1C(=O)NC21CCCCC1] |
| 590 | 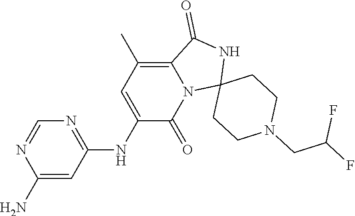 [SMILES: Cc1cc(Nc2cc(N)ncn2)c(=O)n2c1C(=O)NC21CCN(CC(F)F)CC1] |
| 611 | 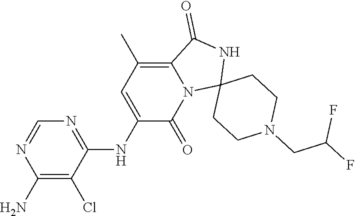 [SMILES: Cc1cc(Nc2ncnc(N)c2Cl)c(=O)n2c1C(=O)NC21CCN(CC(F)F)CC1] |
| 622 | 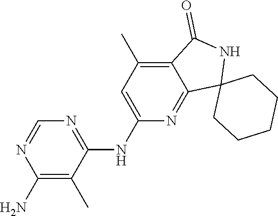 [SMILES: Cc1cc(Nc2ncnc(N)c2C)nc2c1C(=O)NC21CCCCC1] |
| 624 | 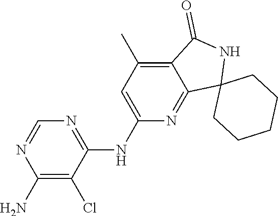 [SMILES: Cc1cc(Nc2ncnc(N)c2Cl)nc2c1C(=O)NC21CCCCC1] |
| 626 | 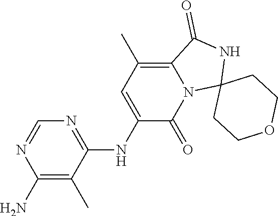 [SMILES: Cc1cc(Nc2ncnc(N)c2C)c(=O)n2c1C(=O)NC21CCOCC1] |
| 637 | 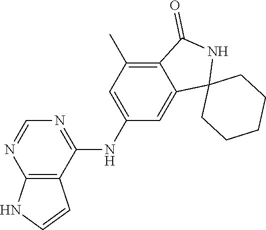 [SMILES: Cc1cc(Nc2ncnc3[nH]ccc23)cc2c1C(=O)NC21CCCCC1] |
| 652 | 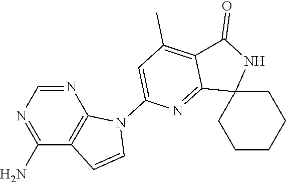 [SMILES: Cc1cc(-n2ccc3c(N)ncnc32)nc2c1C(=O)NC21CCCCC1] |
| 750 | 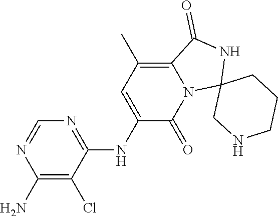 [SMILES: Cc1cc(Nc2ncnc(N)c2Cl)c(=O)n2c1C(=O)NC21CCCNC1] |
| 752 | 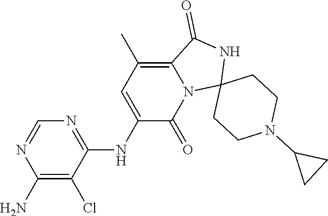 [SMILES: Cc1cc(Nc2ncnc(N)c2Cl)c(=O)n2c1C(=O)NC21CCN(C2CC2)CC1] |
| 753 | 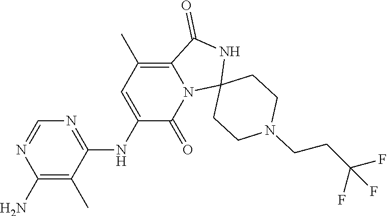 [SMILES: Cc1cc(Nc2ncnc(N)c2C)c(=O)n2c1C(=O)NC21CCN(CCC(F)(F)F)CC1] |
| 775 | 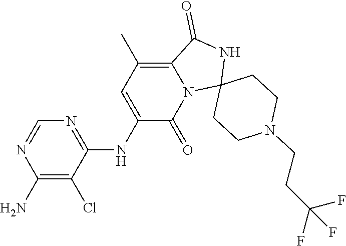 [SMILES: Cc1cc(Nc2ncnc(N)c2Cl)c(=O)n2c1C(=O)NC21CCN(CCC(F)(F)F)CC1] |
| 776 | 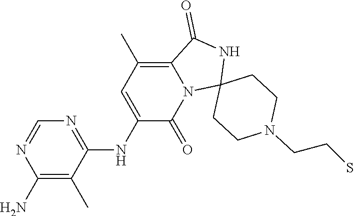 [SMILES: Cc1cc(Nc2ncnc(N)c2C)c(=O)n2c1C(=O)NC21CCN(CCS)CC1] |
| 827 | 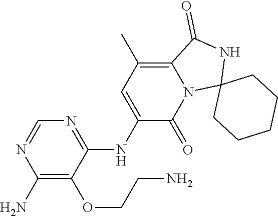 [SMILES: Cc1cc(Nc2ncnc(N)c2OCCN)c(=O)n2c1C(=O)NC21CCCCC1] |
| 917 | 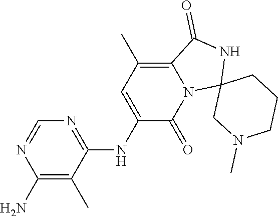 [SMILES: Cc1cc(Nc2ncnc(N)c2C)c(=O)n2c1C(=O)NC21CCCN(C)C1] |
| 969 | 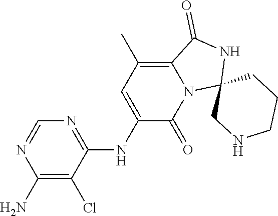 [SMILES: Cc1cc(Nc2ncnc(N)c2Cl)c(=O)n2c1C(=O)N[C@]21CCCNC1] |
| 970 | 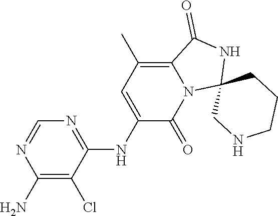 [SMILES: Cc1cc(Nc2ncnc(N)c2Cl)c(=O)n2c1C(=O)N[C@@]21CCCNC1] |
| 1008 | 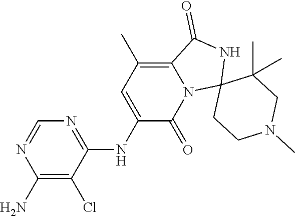 [SMILES: Cc1cc(Nc2ncnc(N)c2Cl)c(=O)n2c1C(=O)NC21CCN(C)CC1(C)C] |
| 1031 | 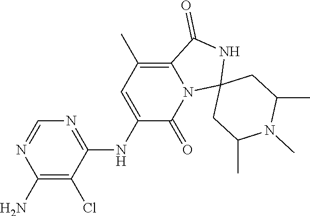 [SMILES: Cc1cc(Nc2ncnc(N)c2Cl)c(=O)n2c1C(=O)NC21CC(C)N(C)C(C)C1] |
| 1053 | 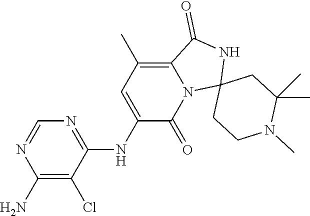 [SMILES: Cc1cc(Nc2ncnc(N)c2Cl)c(=O)n2c1C(=O)NC21CCN(C)C(C)(C)C1] |
| 1090 | 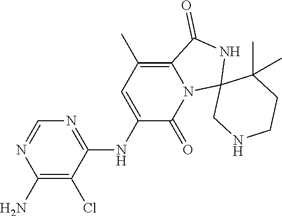 [SMILES: Cc1cc(Nc2ncnc(N)c2Cl)c(=O)n2c1C(=O)NC21CNCCC1(C)C] |
| 1091 | 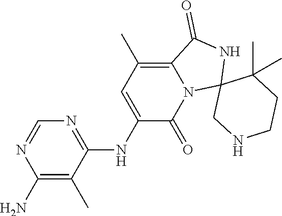 [SMILES: Cc1cc(Nc2ncnc(N)c2C)c(=O)n2c1C(=O)NC21CNCCC1(C)C] |
| 1092 | 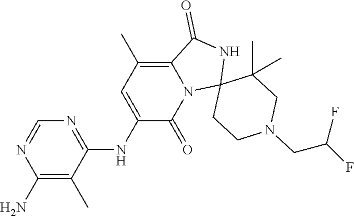 [SMILES: Cc1cc(Nc2ncnc(N)c2C)c(=O)n2c1C(=O)NC21CCN(CC(F)F)CC1(C)C] |

(ii) eIF4A Inhibitors

An “eIF4A inhibitor,” as used herein, refers to an agent or compound that directly interacts with eIF4A, either alone or in a complex (e.g., a ternary complex of an eIF4A inhibitor, an eIF4A and a mRNA) and may block, inactivate, reduce or minimize eIF4A activity (e.g., helicase activity or translational effects), or reduce activity by promoting degradation of eIF4A, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated eIF4A. In certain embodiments, an eIF4A inhibitor is a catalytic inhibitor that directly inhibits eIF4A helicase activity. An example of an eIF4A catalytic inhibitor is BPSL1549, a bacterial toxin from Burkholderia pseudomallei that deamidates Gln339 of eIF4A and converts it into a dominant-negative mutant (Cruz-Migoni et al., Science 334:821-824, 2011, which inhibitor is incorporated herein by reference in its entirety).

In some embodiments, an eIF4A inhibitor is an allosteric inhibitor. An allosteric eIF4A inhibitor binds to eIF4A at a site other than the active site, wherein its binding induces a conformational change in eIF4A so that a substrate can no longer bind eIF4A or eIF4A activity is reduced. In certain embodiments, an allosteric eIF4A inhibitor includes hippuristanol (Bordeleau et al., Nat Chem. Biol. 2: 213-220, 2006, which compound is incorporated herein by reference in its entirety) and derivatives or analogs thereof. Hippuristanol, which binds the C-terminal domain of both free eIF4A (eIF4A_{f}) and eIF4A bound in an eIF4F complex (eIF4A_{c}), inhibits eIF4A helicase and ATPase activities.

In further embodiments, an eIF4A inhibitor is a chemical inducer of dimerization. An eIF4A chemical inducer of dimerization causes a non-sequence specific interaction between eIF4A_{f} and RNA and stimulates the ATP hydrolysis activity of eIF4A, resulting in sequestering of eIF4A_{f} and depletion of eIF4A_{c}. Examples of eIF4A inhibitors that are chemical inducers of dimerization include pateamine A, and analogs, derivatives, or precursors thereof. Examples of pateamine A derivatives have been described in U.S. Pat. No. 7,230,021; PCT Publication WO 2016/161168 (α-amino derivatives that lack the C₅-methyl group); and U.S. Pat. No. 7,737,134 (desmethyl, desamino-pateamine A derivatives), each derivative of which is incorporated by reference in its entirety.

In still further embodiments, an eIF4A inhibitor is a site-directed eIF4A inhibitor. A “site-directed eIF4A inhibitor,” as used herein, refers to an agent or compound that interacts with a specific nucleotide sequence of a mRNA molecule, such as a non-coding nucleotide sequence (e.g., located in the 5′-UTR of a target mRNA), and is capable of forming a stable ternary complex comprised of the site-directed eIF4A inhibitor, an eIF4A and a target mRNA. Exemplary site-directed eIF4A inhibitors include silverstrol, rocaglamide compounds, as well as analogs, derivatives, or precursors thereof. Representative silverstrol derivatives and analogs include CR-1-31-B, hydroxamate derivative of silvestrol (Rodrigo et al., J. Med. Chem. 55:558-562, 2012; which compounds are incorporated herein by reference in their entirety); episilvestrol (Hwang et al., J. Org. Chem. 69:3350-3358, 2004; which compound is incorporated herein by reference in its entirety); Compounds 74 and 76 (Liu et al., J. Med. Chem. 55:8859-8878, 2012, which compounds are incorporated herein by reference in their entirety), silvestrol dioxane, episilvesterol dioxane, Flavagline 61, (−)-4′-desmethoxyepisilvestrol, and 1-O-formylaglafoline. Examples of rocaglates and precursors include aglapervirisin A and aglapervirisins B-J (An et al., Scientific Reports, Article No. 20045, 2016). Further examples of naturally silvestrol and rocaglamide derivatives and analogs are described in Pan et al., Nat. Prod. Rep. 31:924-939, 2014; Kim et al., Anticancer Agents Med. Chem. 6:319-45, 2006; and U.S. Patent Publication US 2014/0255432, compounds from which are incorporated herein by reference in their entirety.

Further examples of site-directed eIF4A inhibitors include compounds as disclosed in PCT Application No. PCT/US2016/063353, which compounds and synthetic methods disclosed therein are incorporated herein by reference in their entirety. In certain embodiments, site-directed eIF4A inhibitors include compounds according to Formula I,

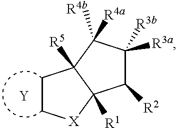

or stereoisomers, tautomers or pharmaceutically acceptable salts thereof, wherein:

X is CR⁶R⁷, O, S, NH, N(C₁-C₈)alkyl, C(O), C═CR⁶R⁷, N(CO)R⁸, S(O) or S(O)₂;

Y is a 5-membered heteroaryl or a 6-membered aryl or heteroaryl;

R¹ and R² independently are aryl, heterocyclyl, heteroaryl or cycloalkyl;

R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹,NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹) (OR⁹), aryl, heteroaryl, cycloalkyl or heterocyclyl;

R^{3a} and R^{3b}, and R^{4a} and R^{4b} independently combine to form oxo or alkenyl, or a cycloalkyl or heterocyclyl ring; or

R^{3a} and R^{4a}, R^{3b} and R^{4b} or R^{4a} and R⁵ together with the carbon atom to which they are attached form a cycloalkyl or heterocyclyl ring; or

R² and R^{3a} together with the carbon atom to which they are attached form a bicyclic ring system;

R⁵ is H, halogen, OH, CN, N₃, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;

R⁶ and R⁷ independently are H, CN, halogen, OR⁹, SR⁹, (C₁-C₈)alkyl, NH(R⁹) or NR⁹R⁹;

R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);

R⁹ is H, (C₁-C₈)alkyl, (C₁-C₈)haloalkyl, cycloalkyl, heterocyclyl, [(C₁-C₈)alkylene]heterocyclyl, aryl, [(C₁-C₈)alkylene]aryl or heteroaryl;

wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;

wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂[CH(OH)]ₘ—(CH₂)ₚ—OH, CH₂— [CH(OH)]ₘ-(CH₂)ₚ—NH₂ or CH₂-aryl-alkoxy; or

wherein any alkyl, cycloalkyl or heterocyclyl is optionally substituted with oxo;

“m” and “p” are 1, 2, 3, 4, 5 or 6; and

wherein when Y is a 6-membered aryl then X is not 0.

In some embodiments, the 6-membered aryl or heteroaryl is

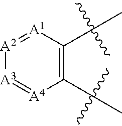

wherein

A¹ is N or CR¹⁰;

A² is N or CR¹¹;

A³ is N or CR¹²;

A⁴ is N or CR¹³; and

R¹⁰, R¹¹, R¹² and R¹³ independently are H, halogen, C₁-C₈(alkyl), (C₁-C₈)haloalkyl, C(O)O(C₁-C₈)alkyl, C(O)(C₁-C₈)alkyl, SO₂(C₁-C₈)alkyl, C₂-C₈(alkenyl), (C₂-C₈)alkynyl, OR⁹, NHR⁹, NR⁹R⁹, CN, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹) (OR⁹), aryl, heteroaryl, cycloalkyl or heterocyclyl.

In certain embodiments, the 5-membered heteroaryl is

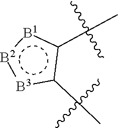

wherein any two of B¹, B² and B³ are CR¹⁴ and N and the remaining B ring atom is N(R¹⁵) or S, wherein R¹⁴ is H, CN, halogen, OR⁹, SR⁹, (C₁-C₈)alkyl, C(O)O(C₁-C₈)alkyl, C(O)(C₁-C₈)alkyl, SO₂(C₁-C₈)alkyl, SO₂NR⁹R⁹, C(O)NR⁹R⁹, NR⁹R⁹ or NR⁹C(O)R⁸, and R¹⁵ is H or (C₁-C₈)alkyl.

In a particular embodiments, eIF4A inhibitor compounds of Formula I are selected from:
Rac-(5aR,6S,7R,8R,8aS)-8,8a-dihydroxy-3-methoxy-5a-(4-methoxyphenyl)-N,N-dimethyl-6-phenyl-5a,7,8,8a-tetrahydro-6H-cyclopenta[4,5]furo[3,2-b]pyridine-7-carboxamide (Cpd. No. 1F),
(5aR,6S,7R,8R,8aS)-3-cyano-5a-(4-cyanophenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 2F),
(5 aR,6S,7R, 8R, 8aS)-3-chloro-5a-(4-cyanophenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 3F),
(5 aR,6S,7R, 8R, 8aS)-3-cyano-8,8a-dihydroxy-5a-(4-methoxyphenyl)-N,N-dimethyl-6-phenyl-5a,7,8,8a-tetrahydro-6H-cyclopenta[4,5]furo[3,2-b]pyridine-7-carboxamide (Cpd. No. 4F),
(5 aR,6S,7R, 8R, 8aS)-3-chloro-8, 8a-dihydroxy-5a-(4-methoxyphenyl)-N,N-dimethyl-6-phenyl-5a,7,8,8a-tetrahydro-6H-cyclopenta[4,5]furo[3,2-b]pyridine-7-carboxamide (Cpd. No. 5F),
(5 aR,6S,7R, 8R, 8aS)-5a-(4-cyanophenyl)-8,8a-dihydroxy-3-methoxy-N,N-dimethyl-6-phenyl-5a,7,8,8a-tetrahydro-6H-cyclopenta[4,5]furo[3,2-b]pyridine-7-carboxamide (Cpd. No. 6F),
(5 aR,6S,7R, 8R, 8aS)-3-chloro-8,8a-dihydroxy-N,N-dimethyl-6-phenyl-5a-(p-tolyl)-5a,7,8,8a-tetrahydro-6H-cyclopenta[4,5]furo[3,2-b]pyridine-7-carboxamide (Cpd. No. 7F),
(5 aR,6S,7R, 8R, 8aS)-3-chloro-8,8a-dihydroxy-N,N-dimethyl-6-phenyl-5a-(4-(trifluoromethyl)phenyl)-5a,7,8,8a-tetrahydro-6H-cyclopenta[4,5]furo[3,2-b]pyridine-7-carboxamide (Cpd. No. 8F),
(5 aR,6S,7R, 8R, 8aS)-5a-(4-cyanophenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 9F),
(5 aR,6S,7R, 8R, 8aS)-3-chloro-5a-(4-fluorophenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 10F),
(5 aR,6S,7R, 8R, 8aS)-3-chloro-5a-(4-chlorophenyl)-8,8a-dihydroxy-N,N-di methyl-6-phenyl-5a,7,8,8a-tetrahydro-6H-cyclopenta[4,5]furo[3,2-b]pyridine-7-carboxamide (Cpd. No. 11F),
(5 aR,6S,7R, 8R, 8aS)-3-chloro-8,8a-dihydroxy-N,N-dimethyl-5a-(4-(methylsulfonyl)phenyl)-6-phenyl-5a,7,8,8a-tetrahydro-6H-cyclopenta[4,5]furo[3,2-b]pyridine-7-carboxamide (Cpd. No. 12F),
Rac-(1R,2R,3S,3 aR, 8bS)-6-cyano-3a-(4-cyanophenyl)-1,8b-dihydroxy-N,N-dimethyl-3-phenyl-2,3,3a, 8b-tetrahydro-1H-benzo[b]cyclopenta[d]thiophene-2-carboxamide (Cpd. No. 13F),
Rac-(5 aR, 6S,7R, 8R, 8aS)-3-cyano-5a-(4-cyanophenyl)-8,8a-dihydroxy-N,N-dimethyl-6-phenyl-5a,7,8,8a-tetrahydro-6H-cyclopenta[4,5]furo[3,2-c]pyridine-7-carboxamide (Cpd. No. 14F),
Rac-(4bS,5R,6R,7S,7 aR)-7a-(4-cyanophenyl)-4b, 5-dihydroxy-2-methoxy-N,N-dimethyl-7-phenyl-4b,6,7,7a-tetrahydro-5H-cyclopenta[4,5]furo[2,3-b]pyridine-6-carboxamide (Cpd. No. 15F),
(5aR,6S,7R,8R,8aS)-3-chloro-5a-(4-(difluoromethyl)phenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 16F),
(5aR,6S,7R,8R,8aS)-3-chloro-8,8a-dihydroxy-N,N-dimethyl-6-phenyl-5a-(4-(trifluoromethoxy)phenyl)-5a,7,8,8a-tetrahydro-6H-cyclopenta[4,5]furo[3,2-b]pyridine-7-carboxamide (Cpd. No. 17F),
(5aR,6S,7R,8R,8aS)-3-chloro-5a-(4-cyanophenyl)-8,8a-dihydroxy-6-phenyl-5a,7,8,8a-tetrahydro-6H-cyclopenta[4,5]furo[3,2-b]pyridine-7-carboxamide (Cpd. No. 18F),
Rac-(5aR,6S,7R,8R,8aS)-5a-(4-cyanophenyl)-8,8a-dihydroxy-1-methoxy-N,N-dimethyl-6-phenyl-5a,7,8,8a-tetrahydro-6H-cyclopenta[4,5]furo[3,2-c]pyridine-7-carboxamide (Cpd. No. 19F),
(5aR,6S,7R,8R,8aS)-3-chloro-5a-(4-cyanophenyl)-8,8a-dihydroxy-N-methyl-6-phenyl-5a,7,8,8a-tetrahydro-6H-cyclopenta[4,5]furo[3,2-b]pyridine-7-carboxamide (Cpd. No. 20F),
Rac-methyl (4aR,5S,6R,7R,7aS)-4a-(4-cyanophenyl)-7,7a-dihydroxy-2-methyl-5-phenyl-2,4a,5,6,7,7a-hexahydrocyclopenta[4,5]furo[3,2-c]pyrazole-6-carboxylate (Cpd. No. 21F),
Rac-(5aR,6S,7R,8S,8aS)-3-chloro-5a-(4-cyanophenyl)-8,8a-dihydroxy-N,N-dimethyl-6-phenyl-5a,7,8,8a-tetrahydro-6H-cyclopenta[4,5]furo[3,2-b]pyridine-7-sulfonamide (Cpd. No. 22F),
(4aR,5S,6R,7R,7aS)-4a-(4-cyanophenyl)-7,7a-dihydroxy-N,N,2-trimethyl-5-phenyl-2,4a,5,6,7,7a-hexahydrocyclopenta[4,5]furo[3,2-c]pyrazole-6-carboxamide (Cpd. No. 23F),
Rac-methyl (5aR,6R,6aS,7aS,7bR)-3-chloro-5a-(4-cyanophenyl)-7b-hydroxy-6-phenyl-5a,7,7a,7b-tetrahydrocyclopropa[4′,5′]cyclopenta[1′,2′:4,5]furo[3,2-b]pyridine-6a(6H)-carboxylate (Cpd. No. 24F),
Rac-methyl (5aR,6R,6aS,7aS,7bR)-3-chloro-5a-(4-cyanophenyl)-7b-hydroxy-6-phenyl-5a,7,7a,7b-tetrahydrocyclopropa[4′,5′]cyclopenta[1′,2′:4,5]furo[3,2-b]pyridine-6a(6H)-carboxylate (Cpd. No. 25F),
Rac-4-((5aR,6S,7R,8R,8aS)-3-chloro-8,8a-dihydroxy-7-(oxazol-2-yl)-6-phenyl-6,7,8,8a-tetrahydro-5aH-cyclopenta[4,5]furo[3,2-b]pyridin-5a-yl)benzonitrile (Cpd. No. 26F),
Rac-(5aR,6S,7R,8R,8aS)-5a-(4-bromophenyl)-3-chloro-8,8a-dihydroxy-6-phenyl-5a,7,8,8a-tetrahydro-6H-cyclopenta[4,5]furo[3,2-b]pyridine-7-carbothioamide (Cpd. No. 27F),
Rac-(5 aR, 6S,7R,8R,8aS)-5a-(4-bromophenyl)-3-chloro-8,8a-dihydroxy-6-phenyl-5a,7,8,8a-tetrahydro-6H-cyclopenta[4,5]furo[3,2-b]pyridine-7-carbothioamide (Cpd. No. 28F),
Rac-(5 aR, 6S,7R,8R,8aS)-5a-(4-cyanophenyl)-3,8,8a-trihydroxy-N,N-dimethyl-6-phenyl-2-(trifluoromethyl)-5a,7,8,8a-tetrahydro-6H-cyclopenta[4,5]furo[3,2-b]pyridine-7-carboxamide (Cpd. No. 29F),
Rac-4-((5aR,6S,7S,8R,8aS)-7-(aminomethyl)-3-chloro-8,8a-dihydroxy-6-phenyl-6,7,8,8a-tetrahydro-5aH-cyclopenta[4,5]furo[3,2-b]pyridin-5a-yl)benzonitrile (Cpd. No. 30F),
Rac-(5aR,6R,6aS,7aS,7bR)-3-chloro-5a-(4-cyanophenyl)-7b-hydroxy-N,N-dimethyl-6-phenyl-5a,7,7a,7b-tetrahydrocyclopropa[4′,5′]cyclopenta[1′,2′:4,5]furo[3,2-b]pyridine-6a(6H)-carboxamide (Cpd. No. 31F),
Rac-(5aR,6S,7R,8aR)-3-chloro-5a-(4-cyanophenyl)-8a-hydroxy-N,N-dimethyl-8-oxo-6-phenyl-5a,7,8,8a-tetrahydro-6H-cyclopenta[4,5]furo[3,2-b]pyridine-7-carboxamide (Cpd. No. 32F),
Rac-(5aR,6S,7R,8R,8aS)-3-chloro-5a-(4-cyanophenyl)-8,8a-dihydroxy-N,N,8-trimethyl-6-phenyl-5a,7,8,8a-tetrahydro-6H-cyclopenta[4,5]furo[3,2-b]pyridine-7-carboxamide (Cpd. No. 33Fa) and Rac-(5aR,6S,7R, 8S,8aS)-3-chloro-5a-(4-cyanophenyl)-8,8a-dihydroxy-N,N, 8-trimethyl-6-phenyl-5a,7,8,8a-tetrahydro-6H-cyclopenta[4,5]furo[3,2-b]pyridine-7-carboxamide (Cpd. No. 33Fb),
Rac-(5aR,6S,8aR)-5a-(4-bromophenyl)-3-chloro-8-methyl ene-6-phenyl-5a,6,7,8-tetrahydro-8aH-cyclopenta[4,5]furo[3,2-b]pyridin-8a-ol (Cpd. No. 34F),
Rac-(5 aR, 6R, 8aS)-5a-(4-bromophenyl)-3-chloro-8a-hydroxy-8-methoxy-N,N-dimethyl-6-phenyl-5a, 8a-dihydro-6H-cyclopenta[4,5]furo[3,2-b]pyridine-7-carboxamide (Cpd. No. 35F),
Rac-(4aR,5S,6R,7R,7aS)-3-chloro-4a-(4-cyanophenyl)-7,7a-dihydroxy-N,N,2-trimethyl-5-phenyl-2,4a,5,6,7,7a-hexahydrocyclopenta[4,5]furo[3,2-c]pyrazole-6-carboxamide (Cpd. No. 36F),
Rac-(4aR,5S,6R,7R,7aS)-4a-(4-bromophenyl)-3-chloro-7,7a-dihydroxy-N,N,2-trimethyl-5-phenyl-2,4a,5,6,7,7a-hexahydrocyclopenta[4,5]furo[3,2-c]pyrazole-6-carboxamide (Cpd. No. 37F),
Rac-(5aR,6S,8R,8aS)-5a-(4-bromophenyl)-3-chloro-6-phenyl-6,7-dihydrospiro[cyclopenta[4,5]furo[3,2-b]pyridine-8,2′-oxetan]-8a(5aH)-ol (Cpd. No. 38F),
Rac-4-((5aR,6S,7 S,8R,8aS)-3-chloro-8,8a-dihydroxy-7-((methylamino)methyl)-6-phenyl-6,7,8,8a-tetrahydro-5aH-cyclopenta[4,5]furo[3,2-b]pyridin-5a-yl)benzonitrile (Cpd. No. 39F),
Rac-4-((5aR,6S,7R,8R,8aS)-3-chloro-7-((dimethylamino)methyl)-8,8a-dihydroxy-6-phenyl-6,7,8,8a-tetrahydro-5aH-cyclopenta[4,5]furo[3,2-b]pyridin-5a-yl)benzonitrile (Cpd. No. 40Fa), rac-4-((5aR,6S,7 S,8R,8aS)-3-chloro-7-((dimethylamino)methyl)-8,8a-dihydroxy-6-phenyl-6,7,8,8a-tetrahydro-5aH-cyclopenta[4,5]furo[3,2-b]pyridin-5a-yl)benzonitrile (Cpd. No. 40Fb), and rac-4-((5aR,6S,7 S,8S,8aS)-3-chloro-7-((dimethylamino)methyl)-8,8a-dihydroxy-6-phenyl-6,7,8,8a-tetrahydro-5aH-cyclopenta[4,5]furo[3,2-b]pyridin-5a-yl)benzonitrile (Cpd. No. 40Fc)
Rac-4-((5aR,6S,7R,8R,8aS)-3-chloro-8,8a-dihydroxy-6-phenyl-7-(pyrrolidin-1-ylmethyl)-6,7,8,8a-tetrahydro-5aH-cyclopenta[4,5]furo[3,2-b]pyridin-5a-yl)benzonitrile (Cpd. No. 41Fa), rac-4-((5 aR, 6S,7 S,8R,8aS)-3-chloro-8,8a-dihydroxy-6-phenyl-7-(pyrrolidin-1-ylmethyl)-6,7,8,8a-tetrahydro-5aH-cyclopenta[4,5]furo[3,2-b]pyridin-5a-yl)benzonitrile (Cpd. No. 41Fb), and rac-4-((5aR,6S,7 S,8S,8aS)-3-chloro-8,8a-dihydroxy-6-phenyl-7-(pyrrolidin-1-ylmethyl)-6,7,8,8a-tetrahydro-5aH-cyclopenta[4,5]furo[3,2-b]pyridin-5a-yl)benzonitrile (Cpd. No. 41Fc),
Rac-(1R,2R,3S,3aR,8bS)-8b-azido-1-hydroxy-6-methoxy-3a-(4-methoxyphenyl)-N,N-dimethyl-3-phenyl-2,3-dihydro-1H-cyclopenta[b.]benzofuran-2-carboxamide (Cpd. No. 42F),
Rac-methyl (5aR,6S,7R,8R,8aS)-5a-(4-bromophenyl)-3-chloro-8a-fluoro-8-hydroxy-6-phenyl-5a,7,8,8a-tetrahydro-6H-cyclopenta[4,5]furo[3,2-b]pyridine-7-carboxylate (Cpd. No. 43F),
Rac-(1R,2R,3S,3 aR, 8bS)-8b-amino-1-hydroxy-6-methoxy-3a-(4-methoxyphenyl)-N,N-dimethyl-3-phenyl-2,3,3a, 8b-tetrahydro-1H-cyclopenta[b]b enz ofuran-2-carbox amide (Cpd. No. 44F),
Rac-(1R,2R,3S,3aR,8bS)-8b-acetamido-1-hydroxy-6-methoxy-3a-(4-methoxyphenyl)-N,N-dimethyl-3-phenyl-2,3,3a,8b-tetrahydro-1H-cyclopenta[b]benzofuran-2-carboxamide (Cpd. No. 45F),
Rac-dimethyl 2-[[(5aR,6S,7R,8aR)-5a-(4-bromophenyl)-3-chloro-8a-hydroxy-8-oxo-6-phenyl-6,7-dihydrocyclopenta[4,5]furo[1,2-b]pyridin-7-yl]methyl]propanedioate (Cpd. No. 46F),
Rac-(5aR,6S,8S,8aR)-5a-(4-bromophenyl)-3-chloro-8a-hydroxy-6-phenyl-5a,7,8,8a-tetrahydro-6H-cyclopenta[4,5]furo[3,2-b]pyridine-8-carbonitrile (Cpd. No. 47F),
Rac-(5aR,6S,8aR)-5a-(4-bromophenyl)-3-chloro-8-ethynyl-6-phenyl-5a,6,7,8-tetrahydro-8aH-cyclopenta[4,5]furo[3,2-b]pyridin-8a-ol (Cpd. No. 48F),
Rac-methyl (5aR,6S,7R,8R,8aR)-5a-(4-bromophenyl)-3-chloro-8-cyano-8a-hydroxy-6-phenyl-5a,7,8,8a-tetrahydro-6H-cyclopenta[4,5]furo[3,2-b]pyridine-7-carboxylate (Cpd. No. 49F),
Rac-methyl (5 aR,6S,7R, 8R, 8aR)-3-chloro-8-cyano-5a-(4-cyanophenyl)-8a-hydroxy-6-phenyl-5a,7,8,8a-tetrahydro-6H-cyclopenta[4,5]furo[3,2-b]pyridine-7-carboxylate (Cpd. No. 50F),
Rac-(5aR,6S,7R,8R,8aR)-3-chloro-8-cyano-5a-(4-cyanophenyl)-8a-hydroxy-N,N-dimethyl-6-phenyl-5a,7,8,8a-tetrahydro-6H-cyclopenta[4,5]furo[3,2-b]pyridine-7-carboxamide (Cpd. No. 51F),
Rac-(5aR,6S,7R,8R,8aR)-3-chloro-8-cyano-5a-(4-cyanophenyl)-8a-hydroxy-6-phenyl-5a,7,8,8a-tetrahydro-6H-cyclopenta[4,5]furo[3,2-b]pyridine-7-carboxamide (Cpd. No. 52F),
Rac-(3aR,3bS,8aR,9R,9aR)-8a-(4-bromophenyl)-6-chloro-3b-hydroxy-9-phenyl-1,3a,3b,8a,9,9a-hexahydro-2H-oxazolo[4″,5″:4′,5′]cyclopenta[1′,2′:4,5]furo[3,2-b]pyridin-2-one (Cpd. No. 53F),
Rac-4-((3aR,3bS,8aR,9R,9aR)-6-chloro-3b-hydroxy-2-oxo-9-phenyl-1,2,3a,3b,9,9a-hexahydro-8aH-oxazolo[4″,5″:4′,5′]cyclopenta[1′,2′:4,5]furo[3,2-b]pyridin-8a-yl)benzonitrile (Cpd. No. 54F),
Rac-(5aR,6S,7 S,8R,8aS)-5a-(4-bromophenyl)-3-chloro-7-(hydroxymethyl)-6-phenyl-5a,6,7,8-tetrahydro-8aH-cyclopenta[4,5]furo[3,2-b]pyridine-8,8a-diol (Cpd. No. 55F),
Rac-4-((5aR,6S,7 S, 8R, 8 aS)-3-chloro-8,8a-dihydroxy-7-(hydroxymethyl)-6-phenyl-6,7,8,8a-tetrahydro-5 aH-cyclopenta[4,5]furo[3,2-b]pyridin-5a-yl)benzonitrile (Cpd. No. 56F),
Rac-(5 aR, 6S,7R, 8R, 8 aS)-5a-(4-bromophenyl)-3-chloro-7-methyl-6-phenyl-5a, 6,7,8-tetrahydro-8 aH-cyclopenta[4,5]furo[3,2-b]pyridine-8,8a-diol (Cpd. No. 57F),
Rac-methyl (5 aR,6S,8S, 8 aS)-5a-(4-bromophenyl)-3-chloro-7-fluoro-8,8a-dihydroxy-6-phenyl-5a,7,8,8a-tetrahydro-6H-cyclopenta[4,5]furo[3,2-b]pyridine-7-carboxylate (Cpd. No. 58F),
Rac-methyl (5 aR,6S,8S, 8 aS)-3-chloro-5a-(4-cyanophenyl)-7-fluoro-8,8a-dihydroxy-6-phenyl-5a,7,8,8a-tetrahydro-6H-cyclopenta[4,5]furo[3,2-b]pyridine-7-carboxylate (Cpd. No. 59F),
Rac-(2aS,3S,3aR,8bS,8cR)-3a-(4-bromophenyl)-6-chloro-3-phenyl-2a,3,3a,8c-tetrahydrooxeto[3″,2″:4′,5′]cyclopenta[1′,2′:4,5]furo[3,2-b]pyridin-8b(2H)-ol (Cpd. No. 60F),
Rac-(2aS,3S,3aR,8bS,8cR)-3a-(4-bromophenyl)-6-chloro-3-phenyl-2a,3,3a,8c-tetrahydrooxeto[3″,2″:4′,5′]cyclopenta[1′,2′:4,5]furo[3,2-b]pyridin-8b(2H)-ol (Cpd. No. 61F),
Rac-(5 aR, 6S,7 S,8R,8aS)-5a-(4-bromophenyl)-3-chloro-7-(methoxymethyl)-6-phenyl-5a,6,7,8-tetrahydro-8aH-cyclopenta[4,5]furo[3,2-b]pyridine-8,8a-diol (Cpd. No. 62F),
Rac-(1aS,3S,3aR,8bS)-3a-(4-bromophenyl)-6-chloro-3-phenyl-1a,2,3,3a-tetrahydro-oxireno[2″,3″:1′,5′]cyclopenta[1′,2′:4,5]furo[3,2-b]pyridine (Cpd. No. 63F),
(4bS,5R,6R,7 S,7 aR)-7a-(4-Cyanophenyl)-4b,5-dihydroxy-4-methoxy-N,N-dimethyl-7-phenyl-4b,6,7,7a-tetrahydro-5H-cyclopenta[4,5]furo[2,3-c]pyridine-6-carboxamide (Cpd. No. 64F),
Rac-4-((4bS,5R,6S,7 S,7 aR)-6-(aminomethyl)-4b,5-dihydroxy-4-methoxy-7-phenyl-5,6,7,7a-tetrahydro-4bH-cyclopenta[4,5]furo[2,3-c]pyridin-7a-yl)benzonitrile (Cpd. No. 65F),
4-((4bS,5R,6S,7S,7aR)-6-((Dimethylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 66F),
4-((4bS,5R, 6S,7 S,7aR)-4b,5-Dihydroxy-4-methoxy-7-phenyl-6-(piperazin-1-ylmethyl)-4b,5,6,7-tetrahydro-7aH-cyclopenta[4,5]furo[2,3-c]pyridin-7a-yl)benzonitrile (Cpd. No. 67F),
Rac-4-((4bS,5R,6S,7 S,7 aR)-4b,5-dihydroxy-4-methoxy-6-(4-methylpiperazin-1-yl)methyl)-7-phenyl-4b,5,6,7-tetrahydro-7aH-cyclopenta[4,5]furo[2,3-c]pyridin-7a-yl)benzonitrile (Cpd. No. 68F),
Rac-4-((4bS,5R,6S,7 S,7 aR)-4b,5-dihydroxy-4-methoxy-6-((methylamino)methyl)-7-phenyl-4b,5,6,7-tetrahydro-6H-cyclopenta[4,5]furo[2,3-c]pyridin-7a-yl)benzonitrile (Cpd. No. 69F),
Rac-4-((4bS,5R,6S,7 S,7 aR)-6-((ethylamino)methyl)-4b,5-dihydroxy-4-methoxy-7-phenyl-4b,5,6,7-tetrahydro-6H-cyclopenta[4,5]furo[2,3-c]pyridin-7a-yl)cyclohexa-1,3-diene-1-carbonitrile (Cpd. No. 70F),
Rac-44(4bS,5R,6S,7 S,7aR)-6-(azetidin-1-ylmethyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 71F),
Rac-4-((4bS,5R,6S,7 S,7 aR)-4b,5-dihydroxy-4-methoxy-7-phenyl-6-(pyrrolidin-1-ylmethyl)-4b,5,6,7-tetrahydro-7aH-cyclopenta[4,5]furo[2,3-c]pyridin-7a-yl)benzonitrile (Cpd. No. 72F),
4-((4bS,5R, 6S,7 S,7aR)-6-((Diethylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 73F),
Rac-4-((4bS,5R,6S,7S,7aR)-6-((ethyl(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 74F),
Rac-4-((4bS,5R,6S,7 S,7 aR)-4b,5-dihydroxy-6-((2-hydroxyethyl)(methyl)amino)methyl)-4-methoxy-7-phenyl-4b,5,6,7-tetrahydro-7aH-cyclopenta[4,5]furo[2,3-c]pyridin-7a-yl)benzonitrile (Cpd. No. 75F),
Rac-4-((4bS,5R,6S,7S,7aR)-6-((benzyl(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 76F),
Rac-4-((4bS,5R,6S,7S,7aR)-6-((benzylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 77F),
Rac-4-((5R,6S,7 S,7aR)-4b,5-dihydroxy-4-methoxy-7-phenyl-6-(((pyridin-3-ylmethyl)amino)methyl)-4b,5,6,7-tetrahydro-7aH-cyclopenta[4,5]furo[2,3-c]pyridin-7a-yl)benzonitrile (Cpd. No. 78F),
Rac-4-((4bS,5R,6S,7 S,7 aR)-4b,5-dihydroxy-6-((2-hydroxyethyl)amino)methyl)-4-methoxy-7-phenyl-5,6,7,7a-tetrahydro-4bH-cyclopenta[4,5]furo[2,3-c]pyridin-7a-yl)benzonitrile (Cpd. No. 79F),
Rac-(4aR,5 S,6R,7R,7 aS)-4a-(4-cyanophenyl)-7,7a-dihydroxy-2-isopropyl-N,N-dimethyl-5-phenyl-2,4a,5,6,7,7a-hexahydrocyclopenta[4,5]furo[3,2-c]pyrazole-6-carboxamide (Cpd. No. 80F),
4-((3aR,4R,4aR,9bS,9cR)-9b-Hydroxy-9-methoxy-2-oxo-4-phenyl-2,3,3a,4,9b, 9c-hexahydro-4aH-oxazolo[4″,5″:4′,5′]cyclopenta[1′,2′: 4,5]furo[2,3-c]pyridin-4a-yl)benzonitrile (Cpd. No. 81F),
Rac-(4aR,5S,6R,7R,7aS)-3-cyano-4a-(4-cyanophenyl)-7,7a-dihydroxy-N,N,2-tri methyl-5-phenyl-2,4a,5,6,7,7a-hexahydrocyclopenta[4,5]furo[3,2-c]pyrazole-6-carboxamide (Cpd. No. 82F),
4-((5 aR, 6S,7R, 8S, 8 aS)-3-Chloro-8,8a-dihydroxy-6-phenyl-7-(pyrrolidin-1-ylsulfonyl)-6,7,8,8a-tetrahydro-5aH-cyclopenta[4,5]furo[3,2-b]pyridin-5a-yl)benzonitrile (Cpd. No. 83F),
Rac-(5 aR, 6S,7R, 8S, 8 aS)-5a-(4-bromophenyl)-3-chloro-7-(methylsulfonyl)-6-phenyl-5a,6,7,8-tetrahydro-8 aH-cyclopenta[4,5]furo[3,2-b]pyridine-8,8a-diol (Cpd. No. 84F),
Rac-4-((5 aR,6S,7R, 8S, 8 aS)-3-chloro-8,8a-dihydroxy-7-(methylsulfonyl)-6-phenyl-6,7,8,8a-tetrahydro-5aH-cyclopenta[4,5]furo[3,2-b]pyridin-5a-yl)benzonitrile (Cpd. No. 85F),
(5 aR,6S,7R, 8S, 8 aS)-5a-(4-Cyanophenyl)-8,8a-dihydroxy-7-(methylsulfonyl)-6-phenyl-5a,7,8,8a-tetrahydro-6H-cyclopenta[4,5]furo[3,2-b]pyridine-3-carbonitrile (Cpd. No. 86F),
Rac-((4bS,5R,6R,7 S,7 aR)-7a-(4-bromophenyl)-4b, 5-dihydroxy-4-methoxy-7-phenyl-4b, 6,7,7a-tetrahydro-5H-cyclopenta[4,5]furo[2,3-c]pyridin-6-yl)(morpholino)methanone (Cpd. No. 87F),
Rac-(4bS,5R, 6R,7 S,7 aR)-7a-(4-bromophenyl)-4b, 5-dihydroxy-4-methoxy-N-methyl-7-phenyl-N-(2,2,2-tri fluoro ethyl)-4b, 6,7,7a-tetrahydro-5H-cyclopenta[4,5]furo[2,3-c]pyridine-6-carboxamide (Cpd. No. 88F),
Rac-(4bS,5R, 6R,7 S,7aR)-7a-(4-bromophenyl)-N-cyclopropyl-4b,5-dihydroxy-4-methoxy-7-phenyl-4b,6,7,7a-tetrahydro-5H-cyclopenta[4,5]furo[2,3-c]pyridine-6-carboxamide (Cpd. No. 89F),
Rac-(4bS,5R, 6R,7 S,7aR)-7a-(4-bromophenyl)-4b,5-dihydroxy-4-methoxy-7-phenyl-N-(2,2,2-trifluoroethyl)-4b,6,7,7a-tetrahydro-5H-cyclopenta[4,5]furo[2,3-c]pyridine-6-carboxamide (Cpd. No. 90F),
Rac-(5 aR,6S,8S,8aS)-5a-(4-bromophenyl)-3-chloro-7,7-difluoro-6-phenyl-5a,6,7,8-tetrahydro-8aH-cyclopenta[4,5]furo[3,2-b]pyridine-8,8a-diol (Cpd. No. 91F),
Rac-(5aR,6R,8R,8aS)-5a-(4-bromophenyl)-3-chloro-6-phenyl-5a,6-dihydrospiro[cyclopenta[4,5]furo[3,2-b]pyridine-7,1′-cyclopropane]-8,8a(8H)-diol (Cpd. No. 92F),
Rac-(5 aR, 6S,7R, 8S,8aS)-7-(benzylsulfonyl)-5a-(4-bromophenyl)-3-chloro-6-phenyl-5a,6,7,8-tetrahydro-8aH-cyclopenta[4,5]furo[3,2-b]pyridine-8,8a-diol (Cpd. No. 93F),
Rac-4-((5aR,6S,7R,8S,8aS)-7-(benzylsulfonyl)-3-chloro-8,8a-dihydroxy-6-phenyl-6,7,8,8a-tetrahydro-5aH-cyclopenta[4,5]furo[3,2-b]pyridin-5a-yl)benzonitrile (Cpd. No. 94F),
(4bS,5R,6R,7 S,7 aR)-7a-(4-Cyanophenyl)-4b,5-dihydroxy-N,N-dimethyl-7-phenyl-4b,6,7,7a-tetrahydro-5H-cyclopenta[4,5]furo[2,3-c]pyridine-6-carboxamide (Cpd. No. 95F),
Rac-(4bS,5R, 6R,7 S, 7aR)-4-cyano-7a-(4-cyanophenyl)-4b,5-dihydroxy-N,N-dimethyl-7-phenyl-4b,6,7,7a-tetrahydro-5H-cyclopenta[4,5]furo[2,3-c]pyridine-6-carboxamide (Cpd. No. 96F),
Rac-(4bS,5R, 6R,7 S,7aR)-7a-(4-bromophenyl)-4-chloro-4b,5-dihydroxy-N,N-dimethyl-7-phenyl-4b,6,7,7a-tetrahydro-5H-cyclopenta[4,5]furo[2,3-c]pyridine-6-carboxamide (Cpd. No. 97F),
(4bS,5R,6R,7 S, 7aR)-4-Chloro-7a-(4-cyanophenyl)-4b,5-dihydroxy-N,N-dimethyl-7-phenyl-4b,6,7,7a-tetrahydro-5H-cyclopenta[4,5]furo[2,3-c]pyridine-6-carboxamide (Cpd. No. 98F),
Rac-(4aR,5 S,6R,7R,7 aS)-4a-(4-cyanophenyl)-7,7a-dihydroxy-2-(4-methoxybenzyl)-N,N-dimethyl-5-phenyl-2,4a,5,6,7,7a-hexahydrocyclopenta[4,5]furo[3,2-c]pyrazole-6-carboxamide (Cpd. No. 99F),
Rac-(4aR,5S,6R,7R,7aS)-4a-(4-cyanophenyl)-7,7a-dihydroxy-N,N-dimethyl-5-phenyl-2,4a,5,6,7,7a-hexahydrocyclopenta[4,5]furo[3,2-c]pyrazole-6-carboxamide (Cpd. No. 100F),
Rac-(4bS,5S,6R,7 S,7 aR)-7a-(4-bromophenyl)-4-methoxy-6-(methylsulfonyl)-7-phenyl-5,6,7,7a-tetrahydro-4bH-cyclopenta[4,5]furo[2,3-c]pyridine-4b,5-diol (Cpd. No. 101F),
4-((4bS,5S,6R,7 S, 7aR)-4b,5-dihydroxy-4-methoxy-6-(methylsulfonyl)-7-phenyl-4b,5,6,7-tetrahydro-7aH-cyclopenta[4,5]furo[2,3-c]pyridin-7a-yl)benzonitrile (Cpd. No. 102F),
4-((4bS,5R,6S,7S,7aR)-6-((dimethylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 103F),
(5 aR, 6S, 7R, 8R, 8aS)-3-chloro-5a-(4-cyanophenyl)-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 104F),
(5aR,6S,7 S, 8R, 8aS)-5a-(4-cyanophenyl)-7-((dimethylamino)methyl)-8,8a-dihydroxy-6-phenyl-5a,7,8,8a-tetrahydro-6H-cyclopenta[4,5]furo[3,2-b]pyridine-3-carbonitrile (Cpd. No. 105F),
(4bS,5R, 6S, 7S, 7aR)-7a-(4-(difluoromethyl)phenyl)-6-((dimethylamino)methyl)-4-methoxy-7-phenyl-5,6,7,7a-tetrahydro-4bH-cyclopenta[4,5]furo[2,3-c]pyridine-4b,5-diol (Cpd. No. 106F),
(4bS,5R,6S,7S,7aR)-6-((dimethylamino)methyl)-4-methoxy-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 107F),
(5aR,6S, 7R, 8R, 8aS)-3-chloro-5a-(4-(difluoromethyl)phenyl)-7-((dimethylamino)methyl)-6-phenyl-5a,6,7,8-tetrahydro-8aH-cyclopenta[4,5]furo[3,2-b]pyridine-8,8a-diol (Cpd. No. 108F),
(5 aR,6S,7 S,8R,8aS)-3-chloro-7-((dimethylamino)methyl)-6-phenyl-5a-(4-(trifluoromethyl)phenyl)-5a,6,7,8-tetrahydro-8aH-cyclopenta[4,5]furo[3,2-b]pyridine-8,8a-diol (Cpd. No. 109F),
4-((4bS,5R, 6S,7 S, 7aR)-4 b,5-dihydroxy-4-methoxy-6-(morpholinomethyl)-7-phenyl-4 b, 5,6,7-tetrahydro-7aH-cyclopenta[4,5]furo[2,3-c]pyridin-7a-yl)benzonitrile (Cpd. No. 110F),
Rac-(4bS,5R, 6R,7 S,7aR)-7a-(4-bromophenyl)-N-(2,2-difluoroethyl)-4b,5-dihydroxy-4-methoxy-N-methyl-7-phenyl-4b,6,7,7a-tetrahydro-5H-cyclopenta[4,5]furo[2,3-c]pyridine-6-carboxamide (Cpd. No. 111F),
4-((4bS,5R, 6S,7 S,7aR)-6-((2,2-difluoroethyl)(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 112F),
4-((4bS,5R,6S,7S,7aR)-6-(4,4-difluoropiperidin-1-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 113F),
Rac-((1R,5S)-8-azabicyclo[3.2.1]octan-8-yl)((4bS,5R,6R,7S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)methanone (Cpd. No. 114F),
4-((4bS,5R, 6S,7 S,7aR)-6-((2,2-difluoroethyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 115F),
Rac-(4bS,5R, 6R,7 S,7aR)-7a-(4-bromophenyl)-N-(2,2-difluoroethyl)-4b,5-dihydroxy-4-methoxy-7-phenyl-4b,6,7,7a-tetrahydro-5H-cyclopenta[4,5]furo[2,3-c]pyridine-6-carboxamide (Cpd. No. 116F),
Rac-(4bS,5 S,6R,7 S,7 aR)-7a-(4-bromophenyl)-4-methoxy-7-phenyl-6-(2,2,2-trifluoroethyl)sulfonyl)-5,6,7,7a-tetrahydro-4bH-cyclopenta[4,5]furo[2,3-c]pyridine-4b,5-diol (Cpd. No. 117F),
Rac-4-((4bS,5S,6R,7S,7aR)-4b,5-dihydroxy-4-methoxy-7-phenyl-6-(phenylsulfonyl)-4b,5,6,7-tetrahydro-7aH-cyclopenta[4,5]furo[2,3-c]pyridin-7a-yl)benzonitrile (Cpd. No. 118F),

Rac-4-((4bS,5S,6R,7S,7aR)-4b,5-dihydroxy-4-methoxy-7-phenyl-6-(pyridin-2-ylsulfonyl)-4b,5,6,7-tetrahydro-7aH-cyclopenta[4,5]furo[2,3-c]pyridin-7a-yl)benzonitrile (Cpd. No. 119F),
4-((4bR,5R,7 S,7aR)-4b-hydroxy-5-(hydroxymethyl)-4-methoxy-7-phenyl-4b,5,6,7-tetrahydro-7aH-cyclopenta[4,5]furo[2,3-c]pyridin-7a-yl)benzonitrile (Cpd. No. 120F),
Rac-((4bS,5R,6R,7 S,7 aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)(3,3-difluoroazetidin-1-yl)methanone (Cpd. No. 121F),
4-((4bS,5R, 6S,7 S,7aR)-6-((3,3-difluoroazetidin-1-yl)methyl)-4b,5-dihydroxy-4-methoxy-7-phenyl-4b,5,6,7-tetrahydro-7aH-cyclopenta[4,5]furo[2,3-c]pyridin-7a-yl) benzonitrile (Cpd. No. 122F),
Rac-(5aR,6S,7R,8S,8aS)-3-chloro-5a-(4-cyanophenyl)-8,8a-dihydroxy-N-methyl-6-phenyl-5a,7,8,8a-tetrahydro-6H-cyclopenta[4,5]furo[3,2-b]pyridine-7-sulfonamide (Cpd. No. 123F),
Rac-(5aR,6S,7R,8S,8aS)-5a-(4-bromophenyl)-3-chloro-8,8a-dihydroxy-N-methyl-6-phenyl-5a,7,8,8a-tetrahydro-6H-cyclopenta[4,5]furo[3,2-b]pyridine-7-sulfonamide (Cpd. No. 124F),
Rac-(5aR,6S,7R,8S,8aS)-3-chloro-5a-(4-cyanophenyl)-8,8a-dihydroxy-N-methyl-6-phenyl-5a,7,8,8a-tetrahydro-6H-cyclopenta[4,5]furo[3,2-b]pyridine-7-sulfonamide (Cpd No. 125F),
4-((4bS,5S,6R,7S,7aR)-4b,5-dihydroxy-4-methoxy-6-(morpholinosulfonyl)-7-phenyl-4b,5,6,7-tetrahydro-7aH-cyclopenta[4,5]furo[2,3-c]pyridin-7a-yl)benzonitrile (Cpd. No. 126F),
Rac-(5aR,6S,8R,8aS)-5a-(4-bromophenyl)-3-chloro-6-phenyl-5a,6,7,8-tetrahydro-8aH-cyclopenta[4,5]furo[3,2-b]pyridine-8,8a-diol (Cpd. No. 127F),
Rac-4-((5aR,6S,8R,8aS)-3-chloro-8,8a-dihydroxy-6-phenyl-6,7,8,8a-tetrahydro-5aH-cyclopenta[4,5]furo[3,2-b]pyridin-5a-yl)benzonitrile (Cpd. No. 128F),
Rac-(5aR,6S,8aR)-5a-(4-bromophenyl)-3-chloro-8a-hydroxy-6-phenyl-5a,6,7,8a-tetrahydro-8H-cyclopenta[4,5]furo[3,2-b]pyridin-8-one (Cpd. No. 129F),
Rac-(5aR,6S,8S,8aS)-5a-(4-bromophenyl)-3-chloro-6-phenyl-5a,6,7,8-tetrahydro-8aH-cyclopenta[4,5]furo[3,2-b]pyridine-8,8a-diol (Cpd. No. 130F),
Rac-4-((5aR,6S,8S,8aS)-3-chloro-8,8a-dihydroxy-6-phenyl-6,7,8,8a-tetrahydro-5aH-cyclopenta[4,5]furo[3,2-b]pyridin-5a-yl)benzonitrile (Cpd. No. 131F),
Rac-N-((5 aR,6S,8aS)-5a-(4-bromophenyl)-3-chloro-8a-hydroxy-6-phenyl-5a,6,7,8a-tetrahydro-8H-cyclopenta[4,5]furo[3,2-b]pyridin-8-ylidene)-4-methylb enzenesulfonohydrazide (Cpd. No. 132F),
Rac-(5aR,6S,8aR)-5a-(4-bromophenyl)-3-chloro-6-phenyl-5a,6-dihydro-8aH-cyclopenta[4,5]furo[3,2-b]pyridin-8a-ol (Cpd. No. 133F),
Rac-methyl (4bS,5R,6R,7 S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridine-6-carboxylate (Cpd. No. 134F),
Rac-((4bS,5R,6R,7 S,7 aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)(4,4-difluoropiperidin-1-yl)methanone (Cpd. No. 135F),
Rac-methyl (5aR,6S,7R, 8R,8aS)-5a-(4-bromophenyl)-3-chloro-8,8a-dihydroxy-1-methoxy-6-phenyl-5a,7,8,8a-tetrahydro-6H-cyclopenta[4,5]furo[3,2-c]pyridine-7-carboxylate (Cpd. No. 136F),
Rac-(5 aR, 6S,7R, 8R,8aS)-5a-(4-bromophenyl)-3-chloro-8,8a-dihydroxy-1-methoxy-N,N-dimethyl-6-phenyl-5a,7,8,8a-tetrahydro-6H-cyclopenta[4,5]furo[3,2-c]pyridine-7-carboxamide (Cpd. No. 137F),
Rac-(5 aR, 6S,7 S, 8R, 8 aS)-5a-(4-bromophenyl)-3-chloro-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 138F),
(5aR,6S,7 S,8R,8aS)-5a-(4-cyanophenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 139F),
4-((5 aR,6 S,7 S, 8R, 8a S)-3-chloro-7-((dimethylamino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 140F),
Rac-4-((5 aR,6S,7 S, 8R,8aS)-7-((dimethylamino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 141F),
Rac-(5 aR, 6S,7 S,8R,8aS)-3-chloro-5a-(4-chlorophenyl)-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 142F),
Rac-(5 aR, 6S,7 S,8R,8aS)-3-chloro-5a-(4-(difluoromethyl)phenyl)-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 143F),
Rac-(5 aR, 6S,7 S,8R,8aS)-3-chloro-7-((dimethylamino)methyl)-1-methoxy-6-phenyl-5a-(4-(trifluoromethyl)phenyl)-5a,6,7,8-tetrahydro-8aH-cyclopenta[4,5]furo[3,2-c]pyridine-8,8a-diol (Cpd. No. 144F),
(5 aR,6S,7 S,8R,8aS)-5a-(4-chlorophenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 145F),
Rac-(5 aR, 6S,7 S,8R,8aS)-5a-(4-(difluoromethyl)phenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 146F),
(5 aR,6S,7 S,8R,8aS)-7-((dimethylamino)methyl)-8,8a-dihydroxy-1-methoxy-6-phenyl-5a-(4-(trifluoromethyl)phenyl)-5a,7,8,8a-tetrahydro-6H-cyclopenta[4,5]furo[3,2-c]pyridine-3-carbonitrile (Cpd. No. 147F),
Rac-(5 aR, 6S,7 S,8R,8aS)-5a-(4-(difluoromethyl)phenyl)-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 148F),
Rac-(5 aR, 6S,7 S,8R,8aS)-7-((dimethylamino)methyl)-1-methoxy-6-phenyl-5a-(4-(trifluoromethyl)phenyl)-5a,6,7,8-tetrahydro-8aH-cyclopenta[4,5]furo[3,2-c]pyridine-8,8a-diol (Cpd. No. 149F),
Rac-4-((5aR,6S,7 S,8R,8aS)-7-((dimethylamino)methyl)-8,8a-dihydroxy-1-methoxy-3-(methylamino)-6-phenyl-6,7,8,8a-tetrahydro-5aH-cyclopenta[4,5]furo[3,2-c]pyridin-5a-yl)benzonitrile (Cpd. No. 150F),
(5aR,6S,7S,8R,8aS)-5a-(4-Cyanophenyl)-8,8a-dihydroxy-1-methoxy-7-(morpholinomethyl)-6-phenyl-5a,7,8,8a-tetrahydro-6H-cyclopenta[4,5]furo[3,2-c]pyridine-3-carbonitrile (Cpd. No. 151F),
Rac-4-((5aR,6S,7 S,8R,8aS)-3-chloro-8,8a-dihydroxy-1-methoxy-7-(morpholinomethyl)-6-phenyl-6,7,8,8a-tetrahydro-5aH-cyclopenta[4,5]furo[3,2-c]pyridin-5a-yl)benzonitrile (Cpd. No. 152F),
Rac-(5aR,6S,7S,8R,8aS)-5a-(4-cyanophenyl)-7-((3,3-difluoropyrrolidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 153F),
Rac-(5aR,6S,7S,8R,8aS)-5a-(4-cyanophenyl)-7-((3,3-difluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 154F),
Rac-(5aR,6S,7S,8R,8aS)-7-((tert-butylamino)methyl)-5a-(4-cyanophenyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 155F),
Rac-(5aR,6S,7S,8R,8aS)-5a-(4-bromophenyl)-7-((4-fluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 156aF),
Rac-4-((5aR,6S,7S,8R,8aS)-3-chloro-74(4-fluoropiperidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 156bF),
Rac-(5aR,6S,7S,8R,8aS)-5a-(4-cyanophenyl)-74(4-fluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 156cF),
Rac-(5aR,6S,7S,8R,8aS)-5a-(4-bromophenyl)-7-((4,4-difluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 157aF),
Rac-4-((5aR,6S,7S,8R,8aS)-3-chloro-7-((4,4-difluoropiperidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 157bF),
Rac-(5aR,6S,7S,8R,8aS)-5a-(4-cyanophenyl)-7-((4,4-difluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 157cF),
Rac-(5aR,6S,7S,8R,8aS)-5a-(4-bromophenyl)-8,8a-dihydroxy-1-methoxy-6-phenyl-7-(pyrrolidin-1-ylmethyl)-5a,7,8,8a-tetrahydro-6H-cyclopenta[4,5]furo[3,2-c]pyridine-3-carbonitrile (Cpd. No. 158aF),
Rac-4-((5aR,6S,7 S,8R,8aS)-3-chloro-8,8a-dihydroxy-1-methoxy-6-phenyl-7-(pyrrolidin-1-ylmethyl)-6,7,8,8a-tetrahydro-5aH-cyclopenta[4,5]furo[3,2-c]pyridin-5a-yl)benzonitrile (Cpd. No. 158bF),
Rac-(5aR,6S,7S,8R,8aS)-5a-(4-cyanophenyl)-8,8a-dihydroxy-1-methoxy-6-phenyl-7-(pyrrolidin-1-ylmethyl)-5a,7,8,8a-tetrahydro-6H-cyclopenta[4,5]furo[3,2-c]pyridine-3-carbonitrile (Cpd. No. 158cF),
Rac-(5aR,6S,7S,8R,8aS)-5a-(4-bromophenyl)-7-((diethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 159aF),
Rac-4-((5aR,6S,7S,8R,8aS)-3-chloro-7-((diethylamino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 159bF),
Rac-(5aR,6S,7S,8R,8aS)-5a-(4-cyanophenyl)-7-((diethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 159cF),
Rac-(5aR,6S,7R,8S,8aS)-5a-(4-bromophenyl)-3-chloro-6-phenyl-7-(pyridin-2-ylthio)-5a,6,7,8-tetrahydro-8aH-cyclopenta[4,5]furo[3,2-b]pyridine-8,8a-diol (Cpd. No. 160aF),
Rac-(5aR,6S,7R,8R,8aS)-5a-(4-bromophenyl)-3-chloro-6-phenyl-7-(pyridin-2-ylthio)-5a,6,7,8-tetrahydro-8aH-cyclopenta[4,5]furo[3,2-b]pyridine-8,8a-diol (Cpd. No. 160bF),
Rac-methyl (1aS,2R,3S,3aR,8bS)-3a-(4-bromophenyl)-6-chloro-3-phenyl-1a,2,3,3a-tetrahydro-oxireno[2″,3″:1′,5′]cyclopenta[1′,2′:4,5]furo[3,2-b]pyridine-2-carboxylate (Cpd. No. 161F),
Rac-(5aR,6S,8R,8aR)-5a-(4-bromophenyl)-3-chloro-8-(hydroxymethyl)-6-phenyl-5a,6,7,8-tetrahydro-8aH-cyclopenta[4,5]furo[3,2-b]pyridin-8a-ol (Cpd. No. 162F),
Rac-(5aR,6S,7R,8S,8aS)-5a-(4-bromophenyl)-3-chloro-6-phenyl-7-(pyridin-2-ylsulfonyl)-5a,6,7,8-tetrahydro-8aH-cyclopenta[4,5]furo[3,2-b]pyridine-8,8a-diol (Cpd. No. 163F),
Rac-4-((5aR,6S,7R,8S,8aS)-3-chloro-8,8a-dihydroxy-6-phenyl-7-(pyridin-2-ylsulfonyl)-6,7,8,8a-tetrahydro-5aH-cyclopenta[4,5]furo[3,2-b]pyridin-5a-yl)benzonitrile (Cpd. No. 164F),
Rac-(5aR,6S,8S,8aR)-8-(aminomethyl)-5a-(4-bromophenyl)-3-chloro-6-phenyl-5a,6,7,8-tetra-hydro-8aH-cyclopenta[4,5]furo[3,2-b]pyridin-8a-ol (Cpd. No. 165),
Rac-(5 aR, 6S, 8S,8aR)-5a-(4-bromophenyl)-3-chloro-8-(hydroxymethyl)-6-phenyl-5a,6,7,8-tetrahydro-8aH-cyclopenta[4,5]furo[3,2-b]pyridin-8a-ol (Cpd. No. 166F),
Rac-4-((5aR,6S,8R,8aR)-3-chloro-8a-hydroxy-8-(hydroxymethyl)-6-phenyl-6,7,8,8a-tetrahydro-5aH-cyclopenta[4,5]furo[3,2-b]pyridin-5a-yl)benzonitrile (Cpd. No. 167F),
Rac-4-((5aR,6S,8S,8aR)-3-chloro-8a-hydroxy-8-(hydroxymethyl)-6-phenyl-6,7,8,8a-tetrahydro-5aH-cyclopenta[4,5]furo[3,2-b]pyridin-5a-yl)benzonitrile (Cpd. No. 168F),
Rac-(2aR,3S,3aR,8bS,8cR)-3a-(4-bromophenyl)-6-chloro-8b-hydroxy-3-phenyl-3,3a,8b,8c-tetrahydrooxeto[3″,2″:4′,5′]cyclopenta[1′,2′:4,5]furo[3,2-b]pyridin-2(2aH)-one (Cpd. No. 169F),
Rac-(4bR,5R,6R,7 S,7aR)-5-(aminomethyl)-7a-(4-bromophenyl)-6-(hydroxymethyl)-4-methoxy-7-phenyl-5,6,7,7a-tetrahydro-4bH-cyclopenta[4,5]furo[2,3-c]pyridin-4b-ol (Cpd. No. 170F),
Rac-4-((4bR,5R,6R,7 S,7aR)-5-(aminomethyl)-4b-hydroxy-6-(hydroxymethyl)-4-methoxy-7-phenyl-4b,5,6,7-tetrahydro-7aH-cyclopenta[4,5]furo[2,3-c]pyridin-7a-yl)benzonitrile (Cpd. No. 171F),
Rac-(4bR,5R,7 S,7aR)-5-(aminomethyl)-7a-(4-bromophenyl)-4-methoxy-7-phenyl-5,6,7,7a-tetrahydro-4bH-cyclopenta[4,5]furo[2,3-c]pyridin-4b-ol (Cpd. No. 172F),
Rac-4-((4bR,5R,7 S,7 aR)-5-(aminomethyl)-4b-hydroxy-4-methoxy-7-phenyl-4b,5,6,7-tetrahydro-7aH-cyclopenta[4,5]furo[2,3-c]pyridin-7a-yl)benzonitrile (Cpd. No. 173F),
Rac-(5aR,6S,8R,8aR)-8-(aminomethyl)-5a-(4-bromophenyl)-3-chloro-6-phenyl-5a,6,7,8-tetrahydro-8aH-cyclopenta[4,5]furo[3,2-b]pyridin-8a-ol (Cpd. No. 174F),
Rac-4-((5aR,6S,8R,8aR)-8-(aminomethyl)-3-chloro-8a-hydroxy-6-phenyl-6,7,8,8a-tetrahydro-5aH-cyclopenta[4,5]furo[3,2-b]pyridin-5a-yl)benzonitrile (Cpd. No. 175F),
Rac-(5aR,6S,8R,8aR)-8-(aminomethyl)-5a-(4-cyanophenyl)-8a-hydroxy-6-phenyl-5a,7,8,8a-tetrahydro-6H-cyclopenta[4,5]furo[3,2-b]pyridine-3-carbonitrile (Cpd. No. 176F),
Rac-(5aR,6S,8R,8aR)-5a-(4-bromophenyl)-3-chloro-8-(morpholinomethyl)-6-phenyl-5a,6,7,8-tetrahydro-8aH-cyclopenta[4,5]furo[3,2-b]pyridin-8a-ol (Cpd. No. 177F),
Rac-4-((5aR,6S,8R,8aR)-3-chloro-8a-hydroxy-8-(morpholinomethyl)-6-phenyl-6,7,8,8a-tetrahydro-5aH-cyclopenta[4,5]furo[3,2-b]pyridin-5a-yl)benzonitrile (Cpd. No. 178F),
Rac-(4bR,5R,6R,7 S,7aR)-7a-(4-bromophenyl)-6-(hydroxymethyl)-4-methoxy-5-(morpho-linomethyl)-7-phenyl-5,6,7,7a-tetrahydro-4bH-cyclopenta[4,5]furo[2,3-c]pyridin-4b-ol (Cpd. No. 179F),
Rac-4-((4bR,5R,6R,7 S,7aR)-4b-hydroxy-6-(hydroxymethyl)-4-methoxy-5-(morpholino-methyl)-7-phenyl-4b,5,6,7-tetrahydro-7aH-cyclopenta[4,5]furo[2,3-c]pyridin-7a-yl)benzonitrile (Cpd. No. 180F),
Rac-(4bR,5R,6R,7 S,7aR)-7a-(4-bromophenyl)-5-(((2,2-difluoroethyl)amino)methyl)-6-(hydroxymethyl)-4-methoxy-7-phenyl-5,6,7,7a-tetrahydro-4bH-cyclopenta[4,5]furo[2,3-c]pyridin-4b-ol (Cpd. No. 181F),
Rac-4-((4bR,5R,6R,7 S,7 aR)-5-(((2,2-difluoroethyl)amino)methyl)-4b-hydroxy-6-(hydroxyl-methyl)-4-methoxy-7-phenyl-4b,5,6,7-tetrahydro-7a11-cyclopenta[4,5]furo[2,3-c]pyridin-7a-yl)benzonitrile (Cpd. No. 182F),
Rac-(4bR,5R,6R,7 S,7aR)-7a-(4-bromophenyl)-6-(hydroxymethyl)-4-methoxy-5-(4-methylpiperazin-1-yl)methyl)-7-phenyl-5,6,7,7a-tetrahydro-4bH-cyclopenta[4,5]furo[2,3-c]pyridin-4b-ol (Cpd. No. 183F),
Rac-4-((4bR,5R,6R,7 S,7aR)-4b-hydroxy-6-(hydroxymethyl)-4-methoxy-54(4-methyl-piperazin-1-yl)methyl)-7-phenyl-4b,5,6,7-tetrahydro-7aH-cyclopenta[4,5]furo[2,3-c]pyridin-7a-yl)benzonitrile (Cpd. No. 184F),
Rac-(4bR,5R,6R,7 S,7aR)-7a-(4-bromophenyl)-6-(hydroxymethyl)-4-methoxy-5-((oxetan-3-ylamino)methyl)-7-phenyl-5,6,7,7a-tetrahydro-4bH-cyclopenta[4,5]furo[2,3-c]pyridin-4b-01 (Cpd. No. 185F),
Rac-4-((4bR,5R,6R,7 S,7 aR)-4b-hydroxy-6-(hydroxymethyl)-4-methoxy-5-((oxetan-3-ylamino)-methyl)-7-phenyl-4b,5,6,7-tetrahydro-7aH-cyclopenta[4,5]furo[2,3-c]pyridin-7a-yl)-benzonitrile (Cpd. No. 186F),
Rac-(4bR,5R,6R,7 S,7aR)-7a-(4-bromophenyl)-6-(hydroxymethyl)-4-methoxy-7-phenyl-5-(((pyridin-4-ylmethyl)amino)methyl)-5,6,7,7a-tetrahydro-4bH-cyclopenta[4,5]furo[2,3-c]pyridin-4b-ol (Cpd. No. 187F),
Rac-4-((4bR,5R,6R,7 S,7aR)-4b-hydroxy-6-(hydroxymethyl)-4-methoxy-7-phenyl-5-(((pyridin-4-ylmethyl)amino)methyl)-4b,5,6,7-tetrahydro-7aH-cyclopenta[4,5]furo[2,3-c]pyridin-7a-yl)benzonitrile (Cpd. No. 188F),
Rac-4-((5aR,6S,7R,8S,8aS)-3-chloro-8,8a-dihydroxy-6-phenyl-7-(pyridin-2-ylthio)-6,7,8,8a-tetrahydro-5aH-cyclopenta[4,5]furo[3,2-b]pyridin-5a-yl)benzonitrile (Cpd. No. 189F),
Rac-(5 aR, 6S,8aS)-5a-(4-bromophenyl)-3-chloro-8-ethynyl-8,8a-dihydroxy-N,N-dimethyl-6-phenyl-5a,7,8,8a-tetrahydro-6H-cyclopenta[4,5]furo[3,2-b]pyridine-7-carboxamide (Cpd. No. 190F),
Rac-(5 aR, 6S,8aS)-5a-(4-bromophenyl)-3-chloro-8,8a-dihydroxy-N,N-dimethyl-6-phenyl-8-(prop-1-yn-1-yl)-5a, 7,8,8a-tetrahydro-6H-cyclopenta[4,5]furo[3,2-b]pyridine-7-carboxamide (Cpd. No. 191F),
Rac-(4bR,7 S,7 aR)-7a-(4-bromophenyl)-4b-hydroxy-4-methoxy-N,N-dimethyl-5-oxo-7-phenyl-4b,6,7,7a-tetrahydro-5H-cyclopenta[4,5]furo[2,3-c]pyridine-6-carboxamide (Cpd. No. 192F),
Rac-methyl (4bS,5R,6R,7aR)-4b,5-dihydroxy-7a-(4-iodophenyl)-4-methoxy-7-phenyl-4b,6,7,7a-tetrahydro-5H-cyclopenta[4,5]furo[2,3-c]pyridine-6-carboxylate (Cpd. No. 193F),
Rac-4-((4bS,5R,6S,7S,7aR)-6-((4-acetylpiperazin-1-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 194F),
Rac-(5 aR, 6S,7 S, 8R, 8aS)-5a-(4-bromophenyl)-3-chloro-7-(((2,2-difluoroethyl)amino)methyl)-1-methoxy-6-phenyl-5a,6,7,8-tetrahydro-8aH-cyclopenta[4,5]furo[3,2-c]pyridine-8,8a-diol (Cpd. No. 195F),
Rac-4-((5aR,6S,7 S, 8R,8aS)-3-chloro-7-(((2,2-difluoroethyl)amino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 196F),
Rac-(5 aR, 6S,7 S, 8R,8aS)-5a-(4-cyanophenyl)-7-(((2,2-difluoroethyl)amino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 197F), 4-((5 aR, 6S,7 S,8R,8aS)-3-chloro-8,8a-dihydroxy-1-methoxy-7-((4-methylpiperazin-1-yl)methyl)-6-phenyl-6,7,8,8a-tetrahydro-5aH-cyclopenta[4,5]furo[3,2-c]pyridin-5a-yl)benzonitrile (Cpd. No. 198aF),
Rac-(5 aR, 6S,7 S,8R,8aS)-5a-(4-cyanophenyl)-8,8a-dihydroxy-1-methoxy-7-((4-methylpiperazin-1-yl)methyl)-6-phenyl-5a,7,8,8a-tetrahydro-6H-cyclopenta[4,5]furo[3,2-c]pyridine-3-carbonitrile (Cpd. No. 198bF),
Rac-(5aR,6S,7R,8R,8aR)-5a-(4-bromophenyl)-3-chloro-7-(hydroxymethyl)-1-methoxy-8-(morpholinomethyl)-6-phenyl-5a,6,7,8-tetrahydro-8aH-cyclopenta[4,5]furo[3,2-c]pyridin-8a-ol (Cpd. No. 199F),
Rac-(5aR,6S,7R,8R,8aR)-5a-(4-cyanophenyl)-8a-hydroxy-7-(hydroxymethyl)-1-methoxy-8-(morpholinomethyl)-6-phenyl-5a,7,8,8a-tetrahydro-6H-cyclopenta[4,5]furo[3,2-c]pyridine-3-carbonitrile (Cpd. No. 200F),
Rac-(4bR,5R,6R,7 S,7aR)-5-((2-oxa-6-azaspiro[3.3]heptan-6-yl)methyl)-7a-(4-bromophenyl)-6-(hydroxymethyl)-4-methoxy-7-phenyl-5,6,7,7a-tetrahydro-4bH-cyclopenta[4,5]furo[2,3-c]pyridin-4b-ol (Cpd. No. 201F),
Rac-4-((4bR,5R,6R,7 S,7aR)-5-(2-oxa-6-azaspiro[3.3]heptan-6-yl)methyl)-4b-hydroxy-6-(hydroxymethyl)-4-methoxy-7-phenyl-4b,5,6,7-tetrahydro-7aH-cyclopenta[4,5]furo[2,3-c]pyridin-7a-yl)benzonitrile (Cpd. No. 202F),
Rac-(4bR,5R,6R,7 S,7aR)-7a-(4-bromophenyl)-6-(hydroxymethyl)-4-methoxy-5-((((1-methyl-1H-pyrazol-5-yl)methyl)amino)methyl)-7-phenyl-5,6,7,7a-tetrahydro-4bH-cyclopenta[4,5]furo[2,3-c]pyridin-4b-ol (Cpd. No. 203F),
Rac-4-(4bR,5R,6R,7 S,7aR)-4b-hydroxy-6-(hydroxymethyl)-4-methoxy-5-((((1-methyl-1H-pyrazol-5-yl)methyl)amino)methyl)-7-phenyl-4b,5,6,7-tetrahydro-7aH-cyclopenta[4,5]furo[2,3-c]pyridin-7a-yl)benzonitrile (Cpd. No. 204F),
Rac-(4bR,5R,6R,7 S,7aR)-7a-(4-bromophenyl)-5-((dimethylamino)methyl)-6-(hydroxymethyl)-4-methoxy-7-phenyl-5,6,7,7a-tetrahydro-4bH-cyclopenta[4,5]furo[2,3-c]pyridin-4b-ol (Cpd. No. 205F),
Rac-4-((4bR,5R,6R,7 S,7aR)-5-((dimethylamino)methyl)-4b-hydroxy-6-(hydroxymethyl)-4-methoxy-7-phenyl-4b,5,6,7-tetrahydro-7aH-cyclopenta[4,5]furo[2,3-c]pyridin-7a-yl)benzonitrile (Cpd. No. 206F),
Rac-(5 aR, 6S,7 S, 8R,8aS)-5a-(4-cyanophenyl)-7-((3,3-difluoroazetidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 207aF),
Rac-4-((5 aR,6S,7S,8R,8aS)-3-chloro-7-((3,3-difluoroazetidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 207bF),
Rac-(5 aR, 6S,7 S,8R,8aS)-5a-(4-cyanophenyl)-7-((2,2-difluoroethyl)(methyl)amino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 208aF),
Rac-4-((5aR,6S,7 S, 8R,8aS)-3-chloro-7-((2,2-difluoroethyl)(methyl)amino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 208bF),
Rac-(5aR,6S,7R,8R,8aR)-5a-(4-bromophenyl)-3-chloro-8-((dimethylamino)methyl)-7-(hydroxymethyl)-1-methoxy-6-phenyl-5a,6,7,8-tetrahydro-8aH-cyclopenta[4,5]furo[3,2-c]pyridin-8a-ol (Cpd. No. 209F),
Rac-(5aR,6S,7R,8R,8aR)-5a-(4-cyanophenyl)-8-((dimethylamino)methyl)-8a-hydroxy-7-(hydroxymethyl)-1-methoxy-6-phenyl-5a,7,8,8a-tetrahydro-6H-cyclopenta[4,5]furo[3,2-c]pyridine-3-carbonitrile (Cpd. No. 210F),
Rac-(5aR,6S,7S,8R,8aS)-5a-(4-cyanophenyl)-74(3-fluoroazetidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 211aF),
Rac-4-((5aR,6S,7S,8R,8aS)-3-chloro-7-((3-fluoroazetidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 211bF),
(5 aR,6S,7 S,8R,8aS)-7-(Azetidin-1-ylmethyl)-5a-(4-cyanophenyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 212F),
Rac-(5aR,6S,7R,8R,8aR)-5a-(4-cyanophenyl)-8-((4,4-difluoropiperidin-1-yl)methyl)-8a-hydroxy-7-(hydroxymethyl)-1-methoxy-6-phenyl-5a,7,8,8a-tetrahydro-6H-cyclopenta[4,5]furo[3,2-c]pyridine-3-carbonitrile (Cpd. No. 213F),
Rac-(5aR,6S,7S,8R,8aS)-5a-(4-bromophenyl)-3-chloro-7-((3,3-dimethylmorpholino)methyl)-6-phenyl-5a,6,7,8-tetrahydro-8aH-cyclopenta[4,5]furo[3,2-b]pyridine-8,8a-diol (Cpd. No. 214F),
Rac-((5aR,6S,7R,8R,8aS)-5a-(4-bromophenyl)-3-chloro-8,8a-dihydroxy-1-methoxy-6-phenyl-5a,7,8,8a-tetrahydro-6H-cyclopenta[4,5]furo[3,2-c]pyridin-7-yl)(3-(difluoromethyl)azetidin-1-yl)methanone (Cpd. No. 215F),
Rac-((5aR,6S,7R,8R,8aS)-5a-(4-bromophenyl)-3-chloro-8,8a-dihydroxy-1-methoxy-6-phenyl-5a,7,8,8a-tetrahydro-6H-cyclopenta[4,5]furo[3,2-c]pyridin-7-yl)(3-(difluoromethyl)azetidin-1-yl)methanone (Cpd. No. 216F),
Rac-4-((5aR,6S,7 S,8R,8aS)-3-chloro-7-((3-(difluoromethyl)azetidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 217F),
Rac-(5aR,6S,7S,8R,8aS)-5a-(4-cyanophenyl)-74(3-(difluoromethyl)azetidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 218F),
Rac-((5aR,6S,7R,8R,8aS)-5a-(4-bromophenyl)-3-chloro-8,8a-dihydroxy-1-methoxy-6-phenyl-5a,7,8,8a-tetrahydro-6H-cyclopenta[4,5]furo[3,2-c]pyridin-7-yl)(1,1-difluoro-4-azaspiro[2.3]hexan-4-yl)methanone (Cpd. No. 219F),
Rac-(5 aR, 6S,7 S,8R,8aS)-5a-(4-bromophenyl)-3-chloro-7-((1,1-difluoro-4-azaspiro[2.3]hexan-4-yl)methyl)-1-methoxy-6-phenyl-5a,6,7,8-tetrahydro-8aH-cyclopenta[4,5]furo[3,2-c]pyridine-8,8a-diol (Cpd. No. 220F),
Rac-(5aR,6S,7S,8R,8aS)-5a-(4-cyanophenyl)-7-((1,1-difluoro-4-azaspiro[2.3]hexan-4-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 221F),
Rac-4-((5aR,6S,7S,8R,8aS)-3-chloro-7-((1,1-difluoro-4-azaspiro[2.3]hexan-4-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 222F),
Rac-(5aR,6S,7S,8R,8aS)-5a-(4-bromophenyl)-3-chloro-1-methoxy-7-((methylamino)methyl)-6-phenyl-5a,6,7,8-tetrahydro-8aH-cyclopenta[4,5]furo[3,2-c]pyridine-8,8a-diol (Cpd. No. 223F),
Rac-4-((5aR,6S,7S,8R,8aS)-3-chloro-8,8a-dihydroxy-1-methoxy-7-((methylamino)methyl)-6-phenyl-6,7,8,8a-tetrahydro-5aH-cyclopenta[4,5]furo[3,2-c]pyridin-5a-yl)benzonitrile (Cpd. No. 224F),
Rac-(5aR,6S,7S,8R,8aS)-5a-(4-cyanophenyl)-8,8a-dihydroxy-1-methoxy-7-((methylamino)methyl)-6-phenyl-5a,7,8,8a-tetrahydro-6H-cyclopenta[4,5]furo[3,2-c]pyridine-3-carbonitrile (Cpd. No. 225F),
Rac-(4bR,5R,6R,7 S,7 aR)-7a-(4-bromophenyl)-5-((tert-butylamino)methyl)-6-(hydroxymethyl)-4-methoxy-7-phenyl-5,6,7,7a-tetrahydro-4bH-cyclopenta[4,5]furo[2,3-c]pyridin-4b-ol (Cpd. No. 226F),
Rac-(5aR,6S,7R,8S,8aR)-5a-(4-bromophenyl)-3-chloro-7-((dimethylamino)methyl)-8a-hydroxy-6-phenyl-5a,7,8,8a-tetrahydro-6H-cyclopenta[4,5]furo[3,2-b]pyridine-8-carbonitrile (Cpd. No. 227F),
Rac-(5aR,6S,7R,8S,8aR)-3-chloro-5a-(4-cyanophenyl)-7-((dimethylamino)methyl)-8a-hydroxy-6-phenyl-5a,7,8,8a-tetrahydro-6H-cyclopenta[4,5]furo[3,2-b]pyridine-8-carbonitrile (Cpd. No. 228F),
Rac-(5 aR, 6S,7R,8R,8aS)-5a-(4-bromophenyl)-8,8a-dihydroxy-1,3-dimethoxy-N,N-dimethyl-6-phenyl-5a,7,8,8a-tetrahydro-6H-cyclopenta[4,5]furo[3,2-c]pyridine-7-carboxamide (Cpd. No. 229F),
Rac-(5 aR, 6S,7 S,8R,8aS)-5a-(4-bromophenyl)-7-((dimethylamino)methyl)-1,3-dimethoxy-6-phenyl-5a,6,7,8-tetrahydro-8aH-cyclopenta[4,5]furo[3,2-c]pyridine-8,8a-diol (Cpd. No. 230F),
4-((5 aR, 6S,7 S,8R,8aS)-7-((dimethylamino)methyl)-8,8a-dihydroxy-1,3-dimethoxy-6-phenyl-6,7,8,8a-tetrahydro-5aH-cyclopenta[4,5]furo[3,2-c]pyridin-5a-yl)benzonitrile (Cpd. No. 231F),
Rac-4-((5aR,6S,7 S,8R,8aS)-7-((diethylamino)methyl)-8,8a-dihydroxy-1,3-dimethoxy-6-phenyl-6,7,8,8a-tetrahydro-5aH-cyclopenta[4,5]furo[3,2-c]pyridin-5a-yl)benzonitrile (Cpd. No. 232F),
4-((4bS,5R,6S,7S,7aR)-4b,5-dihydroxy-4-methoxy-6-((methyl(2,2,2-trifluoroethyl)amino)methyl)-7-phenyl-4b,5,6,7-tetrahydro-7aH-cyclopenta[4,5]furo[2,3-c]pyridin-7a-yl)benzonitrile (Cpd. No. 233F),
Rac-(4bR,5R,7 S,7aR)-7a-(4-(aminomethyl)phenyl)-5-(hydroxymethyl)-4-methoxy-7-phenyl-5,6,7,7a-tetrahydro-4bH-cyclopenta[4,5]furo[2,3-c]pyridin-4b-ol (Cpd. No. 234F),
Rac-((4bS,5R,6R,7 S,7 aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)(2-oxa-6-azaspiro[3.3]heptan-6-yl)methanone (Cpd. No. 235F),
Rac-4-((4bS,5R,6S,7S,7aR)-6-((2-oxa-6-azaspiro[3.3]heptan-6-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 236F),
Rac-(4bS,5R, 6R,7 S,7aR)-7a-(4-bromophenyl)-4b,5-dihydroxy-4-methoxy-N-methyl-N-(oxetan-3-yl)-7-phenyl-4b,6,7,7a-tetrahydro-5H-cyclopenta[4,5]furo[2,3-c]pyridine-6-carboxamide (Cpd. No. 237F),
Rac-4-((4bS,5R,6S,7 S,7 aR)-4b,5-dihydroxy-4-methoxy-6-((methyl(oxetan-3-yl)amino)methyl)-7-phenyl-4b,5,6,7-tetrahydro-7aH-cyclopenta[4,5]furo[2,3-c]pyridin-7a-yl)benzonitrile (Cpd. No. 238F),
Rac-(4bS,5R, 6R,7 S,7aR)-7a-(4-bromophenyl)-4b,5-dihydroxy-4-methoxy-N-(oxetan-3-yl)-7-phenyl-4b,6,7,7a-tetrahydro-5H-cyclopenta[4,5]furo[2,3-c]pyridine-6-carboxamide (Cpd. No. 239F),
Rac-4-((4bS,5R,6S,7 S,7 aR)-4b,5-dihydroxy-4-methoxy-6-((oxetan-3-ylamino)methyl)-7-phenyl-4b,5,6,7-tetrahydro-7aH-cyclopenta[4,5]furo[2,3-c]pyridin-7a-yl)benzonitrile (Cpd. No. 240F),
Rac-4-((4bS,5R,6S,7 S,7 aR)-4b,5-dihydroxy-4-methoxy-6-(4(1-methyl-1H-pyrazol-5-yl)methyl)amino)methyl)-7-phenyl-4b,5,6,7-tetrahydro-7aH-cyclopenta[4,5]furo[2,3-c]pyridin-7a-yl)benzonitrile (Cpd. No. 241F),
Rac-4-((4bS,5R,6S,7 S,7 aR)-4b,5-dihydroxy-4-methoxy-6-(4(1-methyl-1H-pyrazol-5-yl)methyl)amino)methyl)-7-phenyl-4b,5,6,7-tetrahydro-7aH-cyclopenta[4,5]furo[2,3-c]pyridin-7a-yl)benzonitrile (Cpd. No. 242F),
4-((4bS,5R, 6S,7 S, 7aR)-6-((tert-butyl(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 243F),
Rac-4-((4bS,5R,6S,7 S,7 aR)-6-((cyclopropylamino)methyl)-4b,5-dihydroxy-4-methoxy-7-phenyl-4b,5,6,7-tetrahydro-7a11-cyclopenta[4,5]furo[2,3-c]pyridin-7a-yl)benzonitrile (Cpd. No. 244F),
Rac-(4bS,5R, 6R,7 S,7aR)-7a-(4-bromophenyl)-N-cyclopropyl-4b,5-dihydroxy-4-methoxy-N-methyl-7-phenyl-4b,6,7,7a-tetrahydro-5H-cyclopenta[4,5]furo[2,3-c]pyridine-6-carboxamide (Cpd. No. 245F),
Rac-4-((4bS,5R,6S,7S,7aR)-6-((cyclopropyl(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 246F),
Rac-(4bS,5R, 6R,7 S,7aR)-7a-(4-bromophenyl)-N-(2-fluoroethyl)-4b,5-dihydroxy-4-methoxy-N-methyl-7-phenyl-4b,6,7,7a-tetrahydro-5H-cyclopenta[4,5]furo[2,3-c]pyridine-6-carboxamide (Cpd. No. 247F),
4-((4bS,5R,6S,7S,7aR)-6-(((2-fluoroethyl)(methyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 248F),
Rac-(4bS,5R, 6R,7 S,7aR)-7a-(4-bromophenyl)-4b,5-dihydroxy-4-methoxy-N-methyl-N-((1-methyl-1H-pyrazol-5-yl)methyl)-7-phenyl-4b,6,7,7a-tetrahydro-5H-cyclopenta[4,5]furo[2,3-c]pyridine-6-carboxamide (Cpd. No. 249F),
4-((4bS,5R, 6S,7 S,7aR)-4b,5-dihydroxy-4-methoxy-6-((methyl((1-methyl-1H-pyrazol-5-yl)methyl)amino)methyl)-7-phenyl-4b,5,6,7-tetrahydro-7aH-cyclopenta[4,5]furo[2,3-c]pyridin-7a-yl)benzonitrile (Cpd. No. 250F),
Rac-(4bS,5R, 6R,7 S,7aR)-7a-(4-bromophenyl)-4b,5-dihydroxy-N-(2-hydroxy-2-methylpropyl)-4-methoxy-N-methyl-7-phenyl-4b,6,7,7a-tetrahydro-5H-cyclopenta[4,5]furo[2,3-c]pyridine-6-carboxamide (Cpd. No. 251F),
4-((4bS,5R,6S,7S,7aR)-4b,5-dihydroxy-6-(((2-hydroxy-2-methylpropyl)(methyl)amino)methyl)-4-methoxy-7-phenyl-4b,5,6,7-tetrahydro-7aH-cyclopenta[4,5]furo[2,3-c]pyridin-7a-yl)benzonitrile (Cpd. No. 252F),
Rac-(4bS,5R, 6R,7 S,7aR)-7a-(4-bromophenyl)-4b,5-dihydroxy-N-(2-hydroxy-2-methylpropyl)-4-methoxy-7-phenyl-4b,6,7,7a-tetrahydro-5H-cyclopenta[4,5]furo[2,3-c]pyridine-6-carboxamide (Cpd. No. 253F),
4-((4bS,5R,6S,7S,7aR)-4b,5-dihydroxy-6-((2-hydroxy-2-methylpropyl)amino)methyl)-4-methoxy-7-phenyl-4b,5,6,7-tetrahydro-7aH-cyclopenta[4,5]furo[2,3-c]pyridin-7a-yl)benzonitrile (Cpd. No. 254F),
Rac-(4bS,5R,6S,7S,7aR)-6-((dimethylamino)methyl)-4-methoxy-7-phenyl-7a-(p-tolyl)-5,6,7,7a-tetrahydro-4bH-cyclopenta[4,5]furo[2,3-c]pyridine-4b,5-diol (Cpd. No. 255F),
(4bS,5R,6S,7S,7aR)-6-((dimethylamino)methyl)-4-methoxy-7-phenyl-7a-(p-tolyl)-5,6,7,7a-tetrahydro-4bH-cyclopenta[4,5]furo[2,3-c]pyridine-4b,5-diol (Cpd. No. 256F),
Rac-((4bS,5R,6R,7S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)(piperidin-1-yl)methanone (Cpd. No. 257F),
4-((4bS,5R,6S,7S,7aR)-4b,5-dihydroxy-4-methoxy-7-phenyl-6-(piperidin-1-ylmethyl)-4b,5,6,7-tetrahydro-7aH-cyclopenta[4,5]furo[2,3-c]pyridin-7a-yl)benzonitrile (Cpd. No. 258F),
Rac-44(4bS,5R,6S,7S,7aR)-6-((2-fluoroethyl)amino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 259F),
Rac-((4bS,5R,6R,7S,7aR)-7a-(4-bromophenyl)-4b,5-dihydroxy-4-methoxy-N-(2-methoxyethyl)-N-methyl-7-phenyl-4b,6,7,7a-tetrahydro-5H-cyclopenta[4,5]furo[2,3-c]pyridine-6-carboxamide (Cpd. No. 260F),
4-((4bS,5R,6S,7S,7aR)-4b,5-dihydroxy-4-methoxy-6-((2-methoxyethyl)(methyl)amino)methyl)-7-phenyl-4b,5,6,7-tetrahydro-7aH-cyclopenta[4,5]furo[2,3-c]pyridin-7a-yl)benzonitrile (Cpd. No. 261F),
Rac-(4bS,5R,6R,7S,7aR)-7a-(4-bromophenyl)-4b,5-dihydroxy-4-methoxy-N-(2-methoxyethyl)-7-phenyl-4b,6,7,7a-tetrahydro-5H-cyclopenta[4,5]furo[2,3-c]pyridine-6-carboxamide (Cpd. No. 262F),
4-((4bS,5R,6S,7S,7aR)-4b,5-dihydroxy-4-methoxy-6-((2-methoxyethyl)amino)methyl)-7-phenyl-4b,5,6,7-tetrahydro-7aH-cyclopenta[4,5]furo[2,3-c]pyridin-7a-yl)benzonitrile hydrochloride (Cpd. No. 263F),
Rac-((1R,5S)-3-oxa-8-azabicyclo[3.2.1]octan-8-yl)((4bS,5R,6R,7S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)methanone (Cpd. No. 264F),
4-((4bS,5R,6S,7S,7aR)-6-(((1R,5S)-3-oxa-8-azabicyclo[3.2.1]octan-8-yl) methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 265F),
Rac-((1R,5S)-3-oxa-6-azabicyclo[3.1.1]heptan-6-yl)((4bS,5R,6R,7S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)methanone (Cpd. No. 266F),
Rac-4-((4bS,5R,6S,7S,7aR)-6-((1R,5S)-3-oxa-6-azabicyclo[3.1.1]heptan-6-yl)methyl)-4 b, 5-dihydroxy-4-methoxy-7-phenyl-4b,5,6,7-tetrahydro-7aH-cyclopenta[4,5]furo[2,3-c]pyridin-7a-yl)benzonitrile (Cpd. No. 267F),
4-((4bS,5R,6S,7S,7aR)-6-(((1R,5S)-8-azabicyclo[3.2.1]octan-8-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 268F),
Rac-(3aR,4R,4aR,9bS,9cR)-4a-(4-(difluoromethyl)phenyl)-9b-hydroxy-9-methoxy-4-phenyl-3,3a,4,4a,9b,9c-hexahydro-2H-oxazolo[4″,5″:4′,5′]cyclopenta[1′,2′:4,5]furo[2,3-c]pyridin-2-one (Cpd. No. 269F),
Rac-(4bS,5R, 6R,7R,7aR)-6-amino-7a-(4-(difluoromethyl)phenyl)-4-methoxy-7-phenyl-5,6,7,7a-tetrahydro-4bH-cyclopenta[4,5]furo[2,3-c]pyridine-4b,5-diol (Cpd. No. 270F),
Rac-((4bS,5R,6R,7 S,7 aR)-7a-(4-chlorophenyl)-4b,5-dihydroxy-4-methoxy-N,N-dimethyl-7-phenyl-4b,6,7,7a-tetrahydro-5H-cyclopenta[4,5]furo[2,3-c]pyridine-6-carboxamide (Cpd. No. 271F),
(4bS,5R,6S,7S,7aR)-7a-(4-Chlorophenyl)-6-((dimethylamino)methyl)-4-methoxy-7-phenyl-5,6,7,7a-tetrahydro-4bH-cyclopenta[4,5]furo[2,3-c]pyridine-4b,5-diol (Cpd. No. 272F),
Rac-(6-oxa-3-azabicyclo[3.1.1]heptan-3-yl)((4bS,5R,6R,7S,7aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)methanone (Cpd. No. 273F),
4-((4bS,5R,6S,7S,7aR)-6-(6-oxa-3-azabicyclo[3.1.1]heptan-3-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 274F),
Rac-(3aR,4S,4aR,9bS,9cR)-4a-(4-bromophenyl)-9b-hydroxy-9-methoxy-4-phenyl-3,3a,4,4a,9b,9c-hexahydro-2H-furo[3″,2″:4′,5′]cyclopenta[1′,2′:4,5]furo[2,3-c]pyridin-2-one (Cpd. No. 275F),
Rac-4-((4bS,5R,6R,7 S,7aR)-6-(2-(dimethylamino)ethyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 276F),
Rac-(4bS,5R, 6R,7 S,7 aR)-7a-(4-bromophenyl)-4b,5-dihydroxy-4-methoxy-N-methyl-7-phenyl-N-(pyridin-3-ylmethyl)-4b,6,7,7a-tetrahydro-5H-cyclopenta[4,5]furo[2,3-c]pyridine-6-carboxamide (Cpd. No. 277F),
4-((4bS,5R, 6S,7 S,7aR)-4b,5-dihydroxy-4-methoxy-6-((methyl(pyridin-3-ylmethyl)amino)methyl)-7-phenyl-4b,5,6,7-tetrahydro-7aH-cyclopenta[4,5]furo[2,3-c]pyridin-7a-yl)benzonitrile (Cpd. No. 278F),
Rac-((4bS,5R,6R,7 S,7 aR)-7a-(4-bromophenyl)-4b,5-dihydroxy-4-methoxy-7-phenyl-4b,6,7,7a-tetrahydro-5H-cyclopenta[4,5]furo[2,3-c]pyridin-6-yl)(3,3-difluoropyrrolidin-1-yl)methanone (Cpd. No. 279F),
4-((4bS,5R, 6S,7 S,7aR)-6-((3,3-difluoropyrrolidin-1-yl)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 280F),
Rac-((4bS,5R,6R,7 S,7 aR)-4b,5-dihydroxy-4-methoxy-7-phenyl-7a-(4-(trifluoromethyl)phenyl)-4b,6,7,7a-tetrahydro-5H-cyclopenta[4,5]furo[2,3-c]pyridin-6-yl)(morpholino)methanone (Cpd. No. 281F),
(4bS,5R,6S,7S,7aR)-4-Methoxy-6-(morpholinomethyl)-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 282F),
Rac-(4bS,5R,6S,7S,7aR)-4-methoxy-6-((4-methylpiperazin-1-yl)methyl)-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 283F),
(4bS,5R,6S,7S,7aR)-4-Methoxy-6-((4-methylpiperazin-1-yl)methyl)-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 284F),
Rac-(4bS,5R,6R,7S,7aR)—N-(2,2-difluoroethyl)-4b,5-dihydroxy-4-methoxy-7-phenyl-7a-(4-(trifluoromethyl)phenyl)-4b,6,7,7a-tetrahydro-5H-cyclopenta[4,5]furo[2,3-c]pyridine-6-carboxamide (Cpd. No. 285F),
(4bS,5R,6S,7S,7aR)-6-(((2,2-Difluoroethyl)amino)methyl)-4-methoxy-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 286F),
Rac-4-((4bS,5R,7S,7aR)-4b,5-dihydroxy-4-methoxy-5-(morpholinomethyl)-7-phenyl-4b,5,6,7-tetrahydro-7aH-cyclopenta[4,5]furo[2,3-c]pyridin-7a-yl)benzonitrile (Cpd. No. 287F),
(5aR,6S,7S,8R,8aS)-5a-(4-Cyanophenyl)-7-((3,3-difluoroazetidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 288F),
Rac-(4bR,7S,7aR)-4-methoxy-5-(morpholinomethyl)-7-phenyl-7a-(4-(trifluoromethyl)phenyl)-5,6,7,7a-tetrahydro-4bH-cyclopenta[4,5]furo[2,3-c]pyridin-4b-ol (Cpd. No. 289F),
4-((4bS,5R,6S,7S,7aR)-6-((tert-butylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 290F),
4-((4bS,5R,6S,7S,7aR)-4b,5-dihydroxy-4-methoxy-7-phenyl-6-(((2,2,2-trifluoroethyl)amino)methyl)-4b,5,6,7-tetrahydro-7aH-cyclopenta[4,5]furo[2,3-c]pyridin-7a-yl)benzonitrile (Cpd. No. 291F),
Rac-(5aR,6S,7S,8R,8aS)-7-(aminomethyl)-5a-(4-bromophenyl)-3-chloro-1-methoxy-6-phenyl-5a,6,7,8-tetrahydro-8aH-cyclopenta[4,5]furo[3,2-c]pyridine-8,8a-diol (Cpd. No. 292F),
Rac-(5aR,6S,7S,8R,8aS)-7-(aminomethyl)-5a-(4-cyanophenyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 293F),
Rac-4-((5aR,6S,7S,8R,8aS)-7-(aminomethyl)-3-chloro-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 294F), and
Rac-(5aR,6S,7R,8R,8aS)-5a-(4-bromophenyl)-3-chloro-8,8a-dihydroxy-1-methoxy-6-phenyl-5a,7,8,8a-tetrahydro-6H-cyclopenta[4,5]furo[3,2-c]pyridine-7-carboxylic acid (Cpd. No. 295F).

In another embodiment, the compounds according to Formula I are selected from
(5aR,6S,7S,8R,8aS)-7-((Dimethylamino)methyl)-8,8a-dihydroxy-1-methoxy-6-phenyl-5a-(4-(trifluoromethyl)phenyl)-5a,7,8,8a-tetrahydro-6H-cyclopenta[4,5]furo[3,2-c]pyridine-3-carbonitrile (Cpd. No. 147F),
4-((5aR,6S,7S,8R,8aS)-3-Chloro-8,8a-dihydroxy-1-methoxy-7-((4-methylpiperazin-1-yl)methyl)-6-phenyl-6,7,8,8a-tetrahydro-5aH-cyclopenta[4,5]furo[3,2-c]pyridin-5a-yl)benzonitrile (Cpd. No. 198aF),
(5aR,6S,7S,8R,8aS)-7-(Azetidin-1-ylmethyl)-5a-(4-cyanophenyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 212F),
(5aR,6S,7S,8R,8aS)-5a-(4-Chlorophenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 145F),
Rac-(5aR,6S,7S,8R,8aS)-3-chloro-7-((dimethylamino)methyl)-1-methoxy-6-phenyl-5a-(4-(trifluoromethyl)phenyl)-5a,6,7,8-tetrahydro-8aH-cyclopenta[4,5]furo[3,2-c]pyridine-8,8a-diol (Cpd. No. 144F),
Rac-(5aR,6S,7S,8R,8aS)-3-chloro-5a-(4-(difluoromethyl)phenyl)-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 143F),
Rac-(5 aR, 6S,7 S,8R,8aS)-3-chloro-5a-(4-chlorophenyl)-7-((dimethylamino)methyl)-1-methoxy-6-phenyl-5a,6,7,8-tetrahydro-8aH-cyclopenta[4,5]furo[3,2-c]pyridine-8,8a-diol (Cpd. No. 142F),
Rac-4-((5aR,6S,7 S, 8R,8aS)-3-chloro-7-(((2,2-difluoroethyl)amino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 196F),
(5 aR,6S,7 S,8R,8aS)-5a-(4-Cyanophenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 139F),
Rac-4-((5 aR,6S,7 S, 8R,8aS)-3-chloro-7-((3,3-difluoroazetidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 207bF),
Rac-4-((5aR,6S,7 S, 8R,8aS)-3-chloro-8,8a-dihydroxy-1-methoxy-7-(morpholinomethyl)-6-phenyl-6,7,8,8a-tetrahydro-5aH-cyclopenta[4,5]furo[3,2-c]pyridin-5a-yl)benzonitrile (Cpd. No. 152F),
Rac-4-((5 aR,6S,7S,8R,8aS)-3-chloro-7-((4,4-difluoropiperidin-1-yl)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 157bF),
Rac-4-((5aR,6S,7 S, 8R,8aS)-3-chloro-8,8a-dihydroxy-1-methoxy-6-phenyl-7-(pyrrolidin-1-ylmethyl)-6,7,8,8a-tetrahydro-5aH-cyclopenta[4,5]furo[3,2-c]pyridin-5a-yl)benzonitrile (Cpd. No. 158bF),
4-((5 aR, 6S,7 S,8R,8aS)-7-((Dimethylamino)methyl)-8,8a-dihydroxy-1,3-dimethoxy-6-phenyl-6,7,8,8a-tetrahydro-5aH-cyclopenta[4,5]furo[3,2-c]pyridin-5a-yl)benzonitrile (Cpd. No. 231F),
Rac-4-((5aR,6S,7 S,8R,8aS)-3-chloro-7-((diethylamino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 159bF),
4-((5 aR, 6S,7 S,8R,8aS)-3-Chloro-7-((dimethylamino)methyl)-8,8a-dihydroxy-1-methoxy-6-phenyl-6,7,8,8a-tetrahydro-5aH-cyclopenta[4,5]furo[3,2-c]pyridin-5a-yl)benzonitrile (Cpd. No. 140F),
Rac-(5 aR, 6S,7 S,8R,8aS)-5a-(4-(difluoromethyl)phenyl)-7-((dimethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 146F),
(5 aR,6S,7 S,8R,8aS)-5a-(4-Cyanophenyl)-8,8a-dihydroxy-1-methoxy-7-(morpholinomethyl)-6-phenyl-5a,7,8,8a-tetrahydro-6H-cyclopenta[4,5]furo[3,2-c]pyridine-3-carbonitrile (Cpd. No. 151F),
Rac-(5 aR, 6S,7 S,8R,8aS)-5a-(4-cyanophenyl)-7-(((2,2-difluoroethyl)amino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 197F),
Rac-(5aR,6S,7S,8R,8aS)-5a-(4-cyanophenyl)-7-((3,3-difluoroazetidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 207aF),
Rac-(5aR,6S,7S,8R,8aS)-5a-(4-cyanophenyl)-7-((4,4-difluoropiperidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 157cF),
Rac-(5aR,6S,7S,8R,8aS)-5a-(4-cyanophenyl)-7-((3,3-difluoropyrrolidin-1-yl)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 153F),
Rac-(5aR,6S,7S,8R,8aS)-5a-(4-cyanophenyl)-7-((diethylamino)methyl)-8,8a-dihydroxy-1-methoxy-6-phenyl-5a,7,8,8a-tetrahydro-6H-cyclopenta[4,5]furo[3,2-c]pyridine-3-carbonitrile (Cpd. No. 159cF),
Rac-(5aR,6S,7S,8R,8aS)-5a-(4-cyanophenyl)-8,8a-dihydroxy-1-methoxy-6-phenyl-7-(pyrrolidin-1-ylmethyl)-5a,7,8,8a-tetrahydro-6H-cyclopenta[4,5]furo[3,2-c]pyridine-3-carbonitrile (Cpd. No. 158cF),
Rac-4-((4bR,5R,6R,7S,7aR)-4b-hydroxy-6-(hydroxymethyl)-4-methoxy-5-(morpholino-methyl)-7-phenyl-4b,5,6,7-tetrahydro-7aH-cyclopenta[4,5]furo[2,3-c]pyridin-7a-yl)benzonitrile (Cpd. No. 180F),
Rac-4-((4bR,5R,6R,7S,7aR)-5-((dimethylamino)methyl)-4b-hydroxy-6-(hydroxymethyl)-4-methoxy-7-phenyl-4b,5,6,7-tetrahydro-7aH-cyclopenta[4,5]furo[2,3-c]pyridin-7a-yl)benzonitrile (Cpd. No. 206F),
4-((4bS,5R,6S,7S,7aR)-6-((Dimethylamino)methyl)-4b,5-dihydroxy-4-methoxy-7-phenyl-4b,5,6,7-tetrahydro-7aH-cyclopenta[4,5]furo[2,3-c]pyridin-7a-yl)benzonitrile (Cpd. No. 66F),
(4bS,5R,6S,7S,7aR)-7a-(4-Chlorophenyl)-6-((dimethylamino)methyl)-4-methoxy-7-phenyl-5,6,7,7a-tetrahydro-4bH-cyclopenta[4,5]furo[2,3-c]pyridine-4b,5-diol (Cpd. No. 272F),
(4bS,5R,6S,7S,7aR)-7a-(4-(Difluoromethyl)phenyl)-6-((dimethylamino)methyl)-4-methoxy-7-phenyl-5,6,7,7a-tetrahydro-4bH-cyclopenta[4,5]furo[2,3-c]pyridine-4b,5-diol (Cpd. No. 106F),
(4bS,5R,6S,7S,7aR)-6-((Dimethylamino)methyl)-4-methoxy-7-phenyl-7a-(4-(trifluoromethyl)phenyl)-5,6,7,7a-tetrahydro-4bH-cyclopenta[4,5]furo[2,3-c]pyridine-4b,5-diol (Cpd. No. 107F),

or any combination of two to four compounds thereof.

In particular embodiments, a site-directed eIF4A inhibitor is a compound according to the following formula:

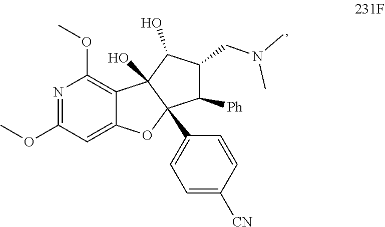

or is a stereoisomer, tautomer, or pharmaceutically acceptable salt thereof.

Methods of testing eIF4A activity are known in the art and include ATPase assays (Pause and Sonenberg, EMBO J. 11:2643-54, 1992; Abramson et al., J. Biol. Chem. 262:3826-3832, 1987; each assay of which is incorporated herein by reference in its entirety), helicase assays (Rogers et al., J. Biol. Chem. 274:12236-44, 1999; Pause and Sonenberg, 1992; each assay of which is incorporated herein by reference in its entirety), and dual luciferase reporter assays (Wolfe et al., Nature 513:65-70, 2014, each assay of which is incorporated herein by reference in its entirety).

(iii) mTOR Inhibitors

An “mTOR inhibitor” refers to an agent or compound that directly interacts with mTOR and may block, inactivate, reduce or minimize mTOR activity (e.g., kinase activity or translational effects), or reduce activity by promoting degradation of mTOR, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated mTOR.

In certain embodiments, a mTOR inhibitor is an allosteric inhibitor. An “allosteric mTOR inhibitor” binds to mTOR at a site other than the active site, wherein its binding induces a conformational change in mTOR so that a substrate can no longer bind mTOR or mTOR activity is reduced. Allosteric mTOR inhibitors include rapamycin (sirolimus), rapamycin-related compounds, that is compounds having structural and functional similarity to rapamycin including, e.g., rapamycin derivatives, rapamycin analogs (also referred to as rapalogs) and other macrolide compounds that inhibit mTOR activity. Examples of allosteric mTOR inhibitors include rapamycin, everolimus, emsirolimus, temsirolimus, umirolimus, ridaforolimus (deforolimus), farnesylthiosalicylic acid, curcumin, and zotarolimus. Further examples of rapamycin analogs include 40-O-benzyl-rapamycin, 40-O-(4′-hydroxymethyl)benzyl-rapamycin, 40-O-[4′-(1,2-dihydroxyethyl)]benzyl-rapamycin, 40-O-allyl-rapamycin, 40-O-[3′-(2,2-dimethyl-1,3-dioxolan-4(S)-yl)-prop-2′-en-yl]-rapamycin, (2′E,4'S)-40-O-(4′,5′-dihydroxypent-2′-en-1′-yl)-rapamycin, 40-O-(2-hydroxy)ethoxycarbonylmethyl-rapamycin, 40-O-(2-hydroxy)ethyl-rapamycin, 40-O-(3-hydroxy)propyl-rapamycin, 40-O-(6-hydroxy)hexyl-rapamycin, 40-O-[2-(2-hydroxy)ethoxy]ethyl-rapamycin, 40-O-[(3S)-2,2-dimethyldioxolan-3-yl]methyl-rapamycin, 40-O-[(2S)-2,3-dihydroxyprop-1-yl]-rapamycin, 40-O-(2-acetoxy)ethyl-rapamycin, 40-O-(2-nicotinoyloxy)ethyl-rapamycin, 40-O-[2-(N-morpholino)acetoxy]ethyl-rapamycin, 40-O-(2-N-imidazolylacetoxy)ethyl-rapamycin, 40-O-[2-(N-methyl-N′-piperazinyl)acetoxy]ethyl-rapamycin, 39-O-desmethyl-39,40-O,O-ethylene-rapamycin, (26R)-26-dihydro-40-O-(2-hydroxy)ethyl-rapamycin, 40-O-(2-aminoethyl)-rapamycin, 40-O-(2-acetaminoethyl)-rapamycin, 40-O-(2-nicotinamidoethyl)-rapamycin, 40-O-(2-(N-methyl-imidazo-2′-ylcarbethoxamido)ethyl)-rapamycin, 40-O-(2-ethoxycarbonylaminoethyl)-rapamycin, 40-O -(2-tolylsulfonamidoethyl)-rapamycin and 40-O-[2-(4′,5′-dicarboethoxy-1′,2′,3′-triazol-F-yl)-ethyl]-rapamycin as disclosed in U.S. Pat. No. 5,665,772 (incorporated by reference in its entirety) and 16-demethoxy-16-(pent-2-ynyl)oxy-rapamycin, 16-demethoxy-16-(but-2-ynyl)oxy-rapamycin, 16-demethoxy-16-(propargyl)oxy-rapamycin, 16-demethoxy-16-(4-hydroxy-but-2-ynyl)oxy-rapamycin, 16-demethoxy-16-benzyloxy-40-O-(2-hydroxyethyl)-rapamycin, 16-demethoxy-16-benzyloxy-rapamycin, 16-demethoxy-16-ortho-methoxybenzyl-rapamycin, 16-demethoxy-40-O-(2-methoxyethyl)-16-pent-2-ynyl)oxy-rapamycin, 39-demethoxy-40-desoxy-39-formyl-42-nor-rapamycin, 39-demethoxy-40-desoxy-39-hydroxymethyl-42-nor-rapamycin, 39-demethoxy-40-desoxy-39-carboxy-42-nor-rapamycin, 39-demethoxy-40-desoxy-39-(4-methyl-piperazin-1-yl)carbonyl-42-nor-rapamycin, 39-demethoxy-40-desoxy-39-(morpholin-4-yl)carbonyl-42-nor-rapamycin, 39-demethoxy-40-desoxy-39-[N-methyl, N-(2-pyridin-2-yl-ethyl)]carbamoyl-42-nor-rapamycin and 39-demethoxy-40-desoxy-39-(p-toluenesulfonylhydrazonomethyl)-42-nor-rapamycin as disclosed in PCT Publication No. WO95/16691 (which compounds are incorporated herein by reference in their entirety), and 32-deoxo-rapamycin, 16-O-pent-2-ynyl-32-deoxo-rapamycin, 16-O-pent-2-ynyl-32-deoxo-40-O-(2-hydroxy-ethyl)-rapamycin, 16-O-pent-2-ynyl-32-(S)-dihydro-40-O-(2-hydroxyethyl)-rapamycin, 32(S)-dihydro-40-O-(2-methoxy)ethyl-rapamycin and 32(S)-dihydro-40-O-(2-hydroxyethyl)-rapamycin as disclosed in PCT Publication No. WO 96/41807 (which compounds are incorporated herein by reference in their entirety).

mTORC1 is sensitive to allosteric mTOR inhibitors such as rapamycin and its derivatives and analogs due to rapamycin's mechanism of action. Rapamycin forms an intracellular complex with intracellular receptor FKBP12. FKBP12-rapamycin complex binds directly to the FKBP12-rapamycin binding domain of mTOR, which is amino terminal to the kinase catalytic domain. This results in a conformational change in mTORC1, which causes the scaffold protein raptor to dissociate from mTOR, in turn blocking its substrates P70 S6 kinase and to a lesser extent 4E-BP1 from accessing mTOR and being phosphorylated. Thus, allosteric mTOR inhibitors inhibit mTOR signaling without altering mTOR's intrinsic catalytic activity. While rapamycin-FKBP12 does not bind to mTORC2, prolonged treatment with rapamycin may inhibit mTORC2 activity indirectly by interfering with assembly of mTORC2 (Sarbassov et al., 2006, Mol. Cell. 22:159-168).

In further embodiments, a mTOR inhibitor is a catalytic inhibitor. A catalytic mTOR inhibitor, also referred to as ATP-competitive mTOR inhibitor, is an agent that directly inhibits the kinase activity of mTORC1, mTORC2, or both, i.e., the agent inhibits phosphorylation activity of mTORC1, mTORC2, or both. Examples of catalytic mTOR inhibitors include BEZ235 (2-methyl-2-[4-(3-methyl-2-oxo-8-quinolin-3-yl-2,3-dihydro-imidazo[4,5-c]quinolin-1-yl)-phenyl]-propionitrile) (described in PCT Publication No. WO2006/122806, which compound is incorporated herein by reference in its entirety), CCG168 (also known as AZD8055, {5-[2,4-bis-((S)-3-methyl-morpholin-4-yl)-pyrido[2,3d]pyrimidin-7-yl]-2-methoxy-phenyl}—methanol) (described in Chresta et al., Cancer Res. 70:288-298, 2010, which compound is incorporated herein by reference in its entirety), PKI-587 (1-[4-[4-(dimethylamino)piperidine-1-carbonyl]phenyl]-3-[4-(4,6-dimorpholino-1,3,5-triazin-2-yl)phenyl]urea) (described in Venkatesan et al., J. Med. Chem. 53:2636-2645, 2010, which compound is incorporated herein by reference in its entirety), GSK-2126458 (2,4-difluoro-N-{2-methoxy-5-[4-(4-pyridazinyl)-6-quinolinyl]-3-pyridinyl}benzenesulfonamide) (Knight et al., ACS Med. Chem. Lett., 2010, 1:39-43, which compound is incorporated herein by reference in its entirety), WYE-354 (described in Yu et al., Cancer Res. 69:6232-6240, 2009, which compound is incorporated herein by reference in its entirety), Ku-0063794 (described in Garcia-Martinez et al., Biochem. J. 421:29-42, 2009, which compound is incorporated herein by reference in its entirety), Ku-0068650 (Malagu et al., Bioorg. Med. Chem. Lett. 19:5950-3, 2009; which compound is incorporated herein by reference in its entirety); torkinib (PP242), sapanisertib (INK128), Torin 1 (1-[4-[4-(1-Oxopropyl)-1-piperazinyl]-3-(trifluoromethyl)phenyl]-9-(3-quinolinyl)-benzo[h]-1,6-naphthyridin-2(1H)-one) (described in Thoreen et al., 2009, J. Biol. Chem. 285:8023-32, which compound is incorporated herein by reference in its entirety), and Torin 2 (9-(6-Amino-3-pyridinyl)-1-[3-(trifluoromethyl)phenyl]-benzo[h]-1,6-naphthyridin-2(1H)-one) (described in Liu et al., Cancer Res. 73:2574-86, 2013, which compound is incorporated herein by reference in its entirety), and AZD2014 (described in Pike et al., 2013, Bioorg. Med. Chem. Lett. 23:1212-6, which compound is incorporated herein by reference in its entirety).

### (iv) RAF Inhibitors

As used herein, a “RAF inhibitor” may block, inactivate, reduce or minimize RAF activity (e.g., kinase activity or translational effects), or reduce activity by promoting degradation of RAF, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated RAF. A RAF inhibitor may inhibit the activity of A-RAF, B-RAF, C-RAF, or any combination thereof. In certain embodiments a RAF inhibitor is a BRAF inhibitor. In certain embodiments, a RAF inhibitor blocks, inactivates, reduces or minimizes the ability of RAF to phosphorylate MEK1/2. Non-limiting examples of RAF inhibitors include TAK-632, HMC95573, TAK-580 (formerly called MLN2480), INU-152, LY3009120, AZ628, LSN3074753, SB590885, CCT196969, CCT241161, DP-4978, (R)-2-(1-(6-amino-5-chloropyrimidine-4-carboxamide)ethyl)-N-(5-chloro-4-(Mfluoromemyl)pyridin-2-yl)thiazole-5-carboxamide, sorafenib, sorafenib tosylate, and lifirafenib.

As used herein, a “BRAF inhibitor” may block, inactivate, reduce or minimize BRAF activity (e.g., kinase activity or translational effects), or reduce activity by promoting degradation of BRAF, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated BRAF. A BRAF inhibitor may be selective for BRAF or may be a pan-RAF inhibitor. In certain embodiments, a BRAF inhibitor blocks, inactivates, reduces or minimizes the ability of BRAF to phosphorylate MEK1/2. In certain embodiments, a BRAF inhibitor targets a V600 mutated BRAF. Non-limiting examples of BRAF inhibitors include encorafenib, vemurafenib, dabrafenib, PLX7904, PLX8394, CEP-32496, GDC-0879, PLX-4720, ZM 336372, GW5074, NVP-BHG712, and RAF265.

### (v) MEK Inhibitors

As used herein, a “MEK inhibitor” may block, inactivate, reduce or minimize MEK1 and/or MEK2 activity (e.g., kinase activity or translational effects), or reduce activity by promoting degradation of MEK1 and/or MEK2, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated MEK1 and/or MEK2. In certain embodiments, a MEK inhibitor blocks, inactivates, reduces or minimizes the ability of MEK to phosphorylate ERK1/2.

Non-limiting examples of MEK inhibitors include trametinib, selumetinib, binimetinib, PD-325901, cobimetinib, CI-1040, MEK162, AZD8330, TAK-733, GDC-0623, refametinib, pimasertib, R04987655, WX-544, HL-085, GDC0973, GSK1 120212, AZD6244, and PD035901.

### (vi) Inhibitors of Immunosuppression Components

In certain embodiments, an additional therapeutic agent that may be used in combination with an eIF4E inhibitor is an inhibitor of an immunosuppression component, which may be an inhibitor of an immune checkpoint molecule or gene, a metabolic enzyme, an immunosuppressive cytokine, T_{reg} cells, or any combination thereof. As used herein, the term “immunosuppression component” refers to one or more cells, proteins, molecules, compounds or complexes providing inhibitory signals to assist in controlling or suppressing an immune response. For example, immunosuppression components include those molecules that partially or totally block immune stimulation; decrease, prevent or delay immune activation; or increase, activate, or up regulate immune suppression. “Controlling or suppressing an immune response,” as used herein, means reducing any one or more of antigen presentation, T cell activation, T cell proliferation, T cell effector function, cytokine secretion or production, and target cell lysis. Such modulation, control or suppression can promote or permit the persistence of a hyperproliferative disease or disorder (e.g., cancer, chronic infections).

Immune checkpoint molecules include immune checkpoint ligands such as, PD-L1, PD-L2, CD80, CD86, B7-H3, B7-H4, HVEM, adenosine, GALS, VISTA, CEACAM-1, CEACAM-3, CEACAM-5, PVRL2, and immune checkpoint receptors such as, PD-1, CTLA-4, BTLA, KIR, LAG3, TIM3, A2aR, CD244/2B4, CD160, TIGIT, LAIR-1, and PVRIG/CD112R). Metabolic enzymes include arginase and indoleamine 2,3-dioxygenase (IDO)), and immunosuppressive cytokines include IL-10, IL-4, IL-1RA, and IL-35. In certain embodiments, an inhibitor of immunosuppression component is a compound, an antisense molecule, a ribozyme, an RNAi molecule (e.g., siRNA), an antibody or antigen binding fragment thereof, or fusion polypeptide (e.g., Fc fusion protein).

An antibody specific for PD-1 may be pidilizumab, nivolumab, pembrolizumab, MEDI0680 (formerly AMP-514), AMP-224, or BMS-936558.

An antibody specific for PD-L1 may be MDX-1105 (BMS-936559), durvalumab (formerly MEDI4736), atezolizumab (formerly MPDL3280A), or avelumab (formerly MSB0010718C). A compound specific for PD-L1 may be BMS-1001 or BMS-1166.

A CTLA4 inhibitor may be a CTLA4 specific antibody, such as tremelimumab or ipilimumab, or a CTLA4-Ig fusion protein (e.g., abatacept, belatacept).

An antibody specific for LAG3 may be relatlimab (BMS-986016), LAG525IMP701, or A9H12. In certain embodiments, a LAG3 inhibitor is a LAG3-Ig fusion protein, such as IMP321.

An IDO inhibitor may be levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, navoximod, GDC-0919, BMS-986205, NLG802, HTI-1090, PF-06840003, 0M2983 (Merck/I0-Met), RG-70099, or any combination thereof.

### D. Methods of Treatment

A wide variety of cancers, including solid tumors and leukemias, are amenable to the methods of treatment disclosed herein. As used, herein the term “cancer” includes solid tumors and hematological malignancies (e.g., leukemias). Exemplary cancers that may be treated include leukemias, lymphomas, myelomas, carcinomas, metastatic carcinomas, sarcomas, adenomas, nervous system cancers and genitourinary cancers. In exemplary embodiments, the methods provided herein are useful in treating adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, and Wilms tumor.

Exemplary hematological malignancies include acute lymphoblastic leukemia (ALL), acute myeloid leukemia (AML), chronic myelogenous leukemia (CML), chronic eosinophilic leukemia (CEL), myelodysplastic syndrome (MDS), Hodgkin's lymphoma, non-Hodgkin's lymphoma (NHL) (e.g., follicular lymphoma, diffuse large B-cell lymphoma, or chronic lymphocytic leukemia), or multiple myeloma (MM).

In a specific embodiment, the BRAF-mutated cancer cells are from a melanoma, lung cancer, small cell lung cancer, non-small-cell lung cancer, head and neck squamous cell carcinoma, sarcoma, thyroid cancer, thyroid carcinoma, colon carcinoma, colorectal cancer, pancreatic cancer, gastric cancer, esophageal cancer, prostate cancer, breast cancer, ovarian cancer, laryngeal cancer, cervical cancer, lymphatic system cancer, genitourinary tract cancer, bone cancer, biliary tract cancer, endometrial cancer, liver cancer, brain cancer, glioblastoma, astrocytoma, ganglioglioma, craniopharyngioma, Langerhans cell histiocytosis, multiple myleoma, leukemia, hairy cell leukemia, or non-Hodgkin's lymphoma cell.

As used herein, a “combination” refers to a combination comprising an eIF4E inhibitor and one or more inhibitors selected from an inhibitor of an immunosuppression component, radiation therapy, surgery, a chemotherapeutic agent, an immunotherapeutic agent targeting an cancer antigen expressed by the tumor (e.g., antibody or adoptive immunotherapeutic agent), a cytokine, an RNA interference agent, or any combination thereof.

In certain embodiments, a chemotherapeutic agent used in combination with an eIF4E inhibitor is an eIF4A inhibitor, a MNK-specific inhibitor, a mTOR inhibitor, a RAF inhibitor, a MEK inhibitor, or any combination thereof. In certain embodiments, the inhibitor of an immunosuppression component is an inhibitor of a PD-L1, PD-L2, CD80, CD86, B7-H3, B7-H4, HVEM, adenosine, GALS, VISTA, CEACAM-1, CEACAM-3, CEACAM-5, and PVRL2), PD-1, CTLA-4, BTLA, KIR, LAG3, TIN/13, A2aR, CD244/2B4, CD160, TIGIT, LAIR-1, PVRIG/CD112R, DO, arginase, TGFβ, IL-10, IL-35, or any combination thereof. Each component of a combination may be administered serially (sequentially), concurrently or simultaneously, with an eIF4E inhibitor as described herein. Such combination therapies may further comprise one or more additional therapeutic agents.

For example, a combination may comprise: (1) an eIF4E inhibitor and a MNK inhibitor; (2) an eIF4E inhibitor and an eIF4A inhibitor; (3) an eIF4E inhibitor and a mTOR inhibitor; (4) an eIF4E inhibitor and a MEK inhibitor; or (5) an eIF4E inhibitor and a RAF inhibitor; and may be optionally combined with: (a) an antibody specific for PD-1, such as pidilizumab, nivolumab, or pembrolizumab; (b) an antibody specific for PD-L1, such as MDX-1105, BMS-936559, MEDI4736, MPDL3280A, or MSB0010718C; or (c) an antibody specific for CTLA4, such as tremelimumab or ipilimumab; each of which may be administered serially (sequentially), concurrently or simultaneously, as described herein. In another example, a combination may comprise: (1) an eIF4E inhibitor and an antibody specific for PD-1, such as pidilizumab, nivolumab, or pembrolizumab; (2) an eIF4E inhibitor and an antibody specific for PD-L1, such as MDX-1105, BMS-936559, MEDI4736, MPDL3280A, or MSB0010718C; or (3) an eIF4E inhibitor and an antibody specific for CTLA4, such as tremelimumab or ipilimumab; and may optionally be combined with (a) a RAF inhibitor; (b) a MEK inhibitor; (c) a mTOR inhibitor; or (d) an eIF4A inhibitor; each of which may be administered serially (sequentially), concurrently or simultaneously, as described herein.

Such combination therapy includes administration of a single pharmaceutical dosage formulation that contains two or more inhibitors, as well as administration of each active agent in its own separate pharmaceutical dosage formulation. For example, a RAF-specific inhibitor, an eIF4A inhibitor, and an eIF4E inhibitor of this disclosure can be administered to the patient together in a single oral dosage composition, such as a tablet or capsule or liquid, or each agent may be administered in separate oral dosage formulations, or each agent may administered by different routes of administration (e.g., oral and parenteral). When separate dosage formulations are used, each of the inhibitors of this disclosure can be administered at essentially the same time, i.e., concurrently, or at separately staggered times, i.e., sequentially; combination therapy is understood to include all these regimens. Administration of an inhibitor(s) of this disclosure may be as a single dose, or administration may occur several times wherein a plurality of doses is given to a subject in need thereof.

Exemplary antibodies that may be used in combination with an eIF4E inhibitor include monoclonal antibodies, antigen binding fragments thereof, or antibody fusion proteins that bind to a cancer antigen (e.g., tumor associated antigen). Exemplary cancer antigens include CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A, mesothelin, NY-ESO-1, PSMA, RANK, ROR1, TNFRSF4, CD40, CD137, TWEAK-R, LTβR, LIFRβ, LRP5, MUC1, OSMRβ, TCRα, TCRβ, CD19, CD20, CD22, CD25, CD28, CD30, CD33, CD52, CD56, CD80, CD81, CD86, CD123, CD171, CD276, B7H4, TLR7, TLR9, PTCH1, PTCH1, Robo1, α-fetoprotein (AFP), Frizzled, OX40 (also referred to as CD134), and CD79b.

Methods of treatment of the present disclosure may further comprise the use of the an adoptive immunotherapeutic agent, such as a T cell containing a chimeric antigen receptor (CAR) or T cell receptor (TCR) specific for a cancer antigen (e.g., a tumor-associated antigen (TAA)), wherein the adoptive immunotherapeutic agent may be administered serially (sequentially), concurrently or simultaneously with the eIF4E inhibitor, as described herein. For example, the eIF4E inhibitor may be administered with a T cell containing a CAR or TCR specific for a cancer antigen (e.g., a tumor-associated antigen (TAA)), such as CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A, mesothelin, NY-ESO-1, PSMA, RANK, ROR1, TNFRSF4, CD40, CD137, TWEAK-R, LTβR, LIFRβ, LRP5, MUC1, OSMRβ, TCRα, TCRβ, CD19, CD20, CD22, CD25, CD28, CD30, CD33, CD52, CD56, CD80, CD81, CD86, CD123, CD171, CD276, B7H4, TLR7, TLR9, PTCH1, PTCH1, Robo1, α-fetoprotein (AFP), Frizzled, OX40 (also referred to as CD134), or CD79b.

In certain embodiments, a combination therapy method comprises administering an eIF4E inhibitor and further administering a radiation treatment or a surgery to a subject. Radiation therapy includes X-ray therapies, such as gamma-irradiation, and radiopharmaceutical therapies. Surgeries and surgical techniques appropriate to treating a given cancer or non-inflamed solid tumor may be used in a subject in combination with an eIF4E inhibitor of this disclosure.

Cytokines can be used to manipulate host immune response towards anticancer activity. See, e.g., Floros and Tarhini, Semin. Oncol. 42:539, 2015. Cytokines useful for promoting anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination.

Another cancer therapy approach involves reducing expression of oncogenes and other genes needed for growth, maintenance, proliferation, and immune evasion by cancer cells. RNA interference agents, such as microRNAs (miRNAs) and small inhibitory RNAs (siRNAs), provide an approach for knocking down expression of cancer genes. See, e.g., Larsson et al., Cancer Treat. Rev. 16:128, 2017.

In certain embodiments, eIF4E inhibitors are formulated as pharmaceutically acceptable compositions that contain an eIF4E inhibitor in an amount effective to inhibit BRAF-mutated cancer cells in a subject. Pharmaceutical compositions for use in methods of the present disclosure can comprise an eIF4E inhibitor in combination with a pharmaceutically acceptable carrier, diluent or excipient.

In this regard, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions disclosed herein can be prepared by combining an eIF4E inhibitor with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, intratumoral, rectal, vaginal, and intranasal. The term “parenteral” as used herein includes subcutaneous injections, intravenous, intramuscular, intrasternal injection or infusion techniques. Pharmaceutical compositions for use in the presently disclosed methods are formulated so as to allow the active ingredients contained therein to be bioavailable upon administration of the composition to a subject. Pharmaceutical compositions that will be administered to a subject may take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an eIF4A inhibitor as described herein, or a pharmaceutically acceptable salt thereof, for use in a method as described herein.

A pharmaceutical composition for use in the present methods may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration. When intended for oral administration, a pharmaceutical composition is preferably in either solid or liquid form, where semi-solid, semi-liquid, suspension and gel forms are included within the forms considered herein as either solid or liquid.

As a solid composition for oral administration, a pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

A pharmaceutical composition may be in the form of a liquid, for example, an elixir, syrup, solution, emulsion or suspension. The liquid may be for oral administration or for delivery by injection, as two examples. When intended for oral administration, a composition may contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration in a method of this disclosure should contain an amount of a compound of the invention such that a suitable dosage will be obtained.

A pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

A pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A pharmaceutical composition may include various materials that modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

A pharmaceutical composition in solid or liquid form may include an agent that binds to the compound of the invention and thereby assists in the delivery of the compound. Suitable agents that may act in this capacity include a monoclonal or polyclonal antibody, a protein or a liposome.

A pharmaceutical composition may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation, may determine preferred aerosols.

Pharmaceutical compositions may be prepared by any methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

eIF4E inhibitors and pharmaceutical compositions of this disclosure may also be useful for the manufacture of a medicament for treating a BRAF-mutated cancer cells or a related cancer, e.g., by promoting apoptosis of BRAF-mutated cancer cells.

In any of the aforementioned embodiments, a pharmaceutical composition as disclosed herein is administered to a subject in an amount sufficient to inhibit activity of each target protein, and preferably with acceptable toxicity to the same. Appropriate concentrations and dosages can be readily determined by one skilled in the art.

The inhibitor(s) described herein is administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

“Effective amount” or “therapeutically effective amount” refers to that amount of an inhibitor described herein which, when administered to a mammal (e.g., human), is sufficient to treat a disease in the mammal, such as a human. The amount of an inhibitor disclosed herein that constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially, concurrently or simultaneously.

The inhibitor(s) and pharmaceutical compositions thereof provided herein are administered to a subject who has or is at risk of developing a cancer at a therapeutically effective amount or dose. Such a dose may be determined or adjusted depending on various factors including the specific therapeutic agents or pharmaceutical compositions, the routes of administration, the subject's condition, that is, stage of the disease, severity of symptoms caused by the disease, general health status, as well as age, gender, and weight, and other factors apparent to a person skilled in the medical art. Similarly, the dose of the therapeutic for treating a disease or disorder may be determined according to parameters understood by a person skilled in the medical art.

Generally, a therapeutic agent is administered at a therapeutically effective amount or dose. A therapeutically effective amount or dose will vary according to several factors, including the chosen route of administration, formulation of the composition, patient response, severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. In addition, a patient may be given a plurality of doses over a determined period of time and in particular time increments (such as daily, weekly, biweekly, monthly, quarterly, biannually, annually or the like). Determination of an effective amount or dosing regimen is well within the capability of those skilled in the art.

When referring to a combination, a therapeutically effective dose refers to combined amounts of the active ingredients that result in the therapeutic effect, whether administered serially or simultaneously (in the same formulation or concurrently in separate formulations). The most effective doses may generally be determined using experimental models and/or clinical trials. Design and execution of pre-clinical and clinical studies for a therapeutic agent (including when administered for prophylactic benefit) described herein are well within the skill of a person skilled in the relevant art.

The route of administration of a therapeutic agent can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

### EXAMPLES

### Example 1

Cell Line Profiling

In order to assess cellular phenotypes arising from eIF4E inhibition, Compound X was profiled in a panel of 100 tumor cell lines. Compound X was profiled using the OncoPanel Cell-Based Profiling Service (Eurofins Pharma Discovery Services, St. Charles, Mo.). Briefly, cell lines used in the panel were grown in RPMI 1640, 10% FBS, 2 mM L-alanyl-L-glutamine, 1 mM Na pyruvate, or a special medium. Cells were seeded into 384-well plates and incubated in a humidified atmosphere of 5% CO₂ at 37° C. Compounds were added the day following cell seeding. At the same time, a time zero untreated cell plate was generated. After a 3-day incubation period, cells were fixed and stained with fluorescently-labeled antibodies and nuclear dye to allow imaging of nuclei, apoptotic cells and mitotic cells.

Compound X having the following structure

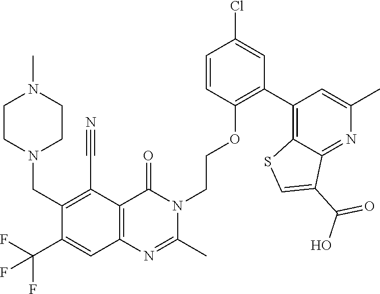 [SMILES: Cc1cc(-c2cc(Cl)ccc2OCCn2c(C)nc3cc(C(F)(F)F)c(CN4CCN(C)CC4)c(C#N)c3c2=O)c2scc(C(=O)O)c2n1]

was serially diluted in half-log steps from 10 μM and assayed over 10 concentrations with a maximum assay concentration of 0.1% DMSO. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

Cell proliferation was measured by the fluorescence intensity of an incorporated nuclear dye. The output is referred to as the relative cell count, where the measured nuclear intensity is transformed to percent of control (POC) using the following formula:

$$POC = \frac{{I}_{x}}{{I}_{0}} \times 100$$

where Iₓ the nuclear intensity at concentration x, and I₀ is the average nuclear intensity of the untreated vehicle wells.

Cellular response parameters were calculated using nonlinear regression to a sigmoidal single-site dose response model:

$$y = A + \frac{B - A}{1 + {( C / x )}^{D}}$$

where y is a response measured at concentration x, A and B are the lower and upper limits of the response, C is the concentration at the response midpoint (EC₅₀), and D is the Hill Slope (Fallahi-Sichani et al., 2013, Nat. Chem. Biol. 9:708-714).

Time zero non-treated plates were used to determine the number of doublings during the assay period, using the formula:

$$Doublings = {log}_{2} ( \frac{N}{{N}_{T 0}} )$$

where N is the cell number in untreated wells at the assay end point and N_{T0} is the cell number at the time of compound addition.

Cell count IC₅₀ is the test compound concentration at 50% of maximal possible response.

An antibody to activated caspase-3 was used to label cells from early to late stage apoptosis (Thornberry et al., 1998, Science 281:1312-1316). The output is shown as a fold increase of apoptotic signal over vehicle background normalized to the relative cell count in each well. A 5-fold induction in the caspase-3 signal was scored as a significant apoptosis induction.

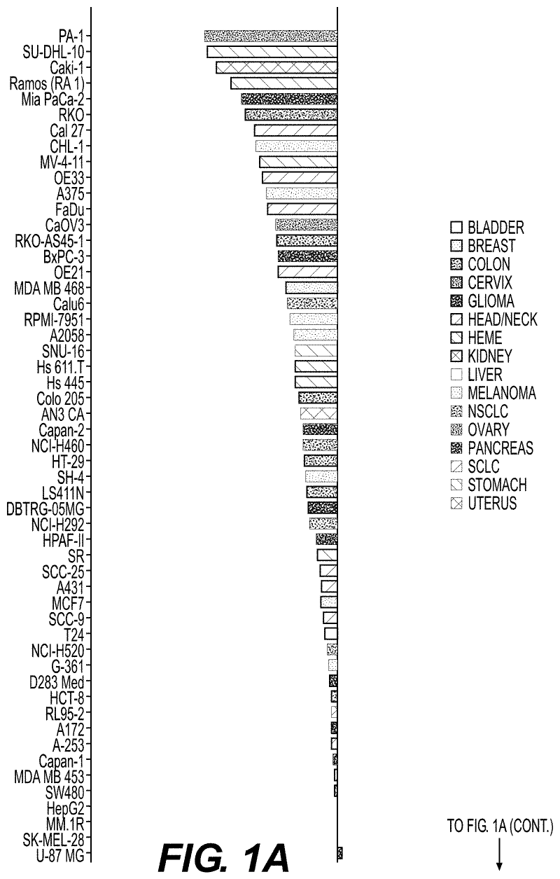

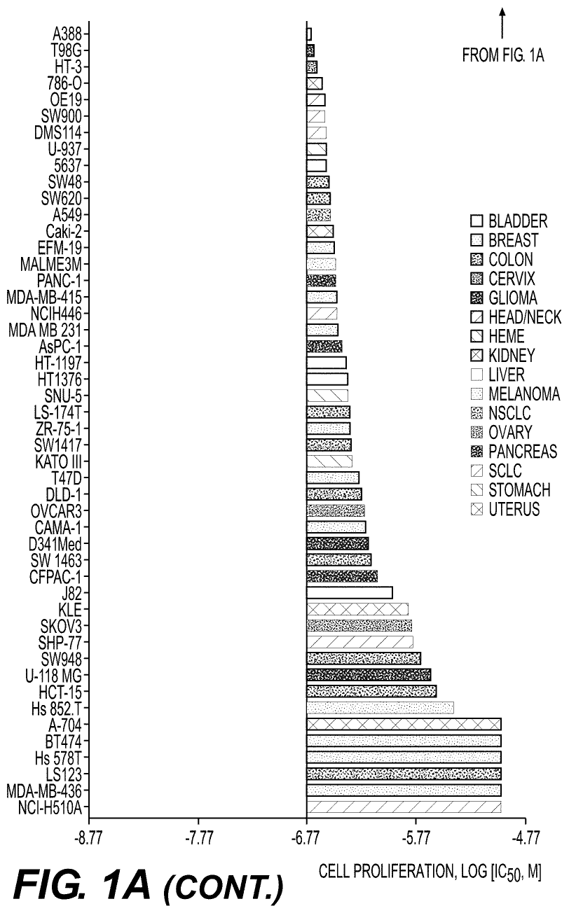

Across the panel of 100 cell lines, the median IC₅₀ value for cell proliferation was 116 nM, with a distribution of IC₅₀ values ranging from 12 nM to >10,000 nM (FIG. 1A). Several trends in Compound X sensitivity were observed when cells were stratified by tumor type. Hematological, head and neck, NSCLC, melanoma, uterine, and pancreatic tumor cell lines were more sensitive to Compound X, where >74% of the cell lines tested in these tumor types had IC₅₀ values below the median (FIG. 1B). Analysis of the mutation status of key cancer-related genes in the tumor cell line panel revealed an additional pattern of interest in the proliferation data. In the melanoma cell lines that had IC₅₀ values below the median, 5 out of the 6 cell lines harbored a BRAF^{V600E} mutation. Furthermore, in colorectal or glioma cell lines which did not show increased sensitivity from a tissue-of-origin standpoint, sensitivity to compound X was enriched in those that were mutant for BRAF^{V600E}.

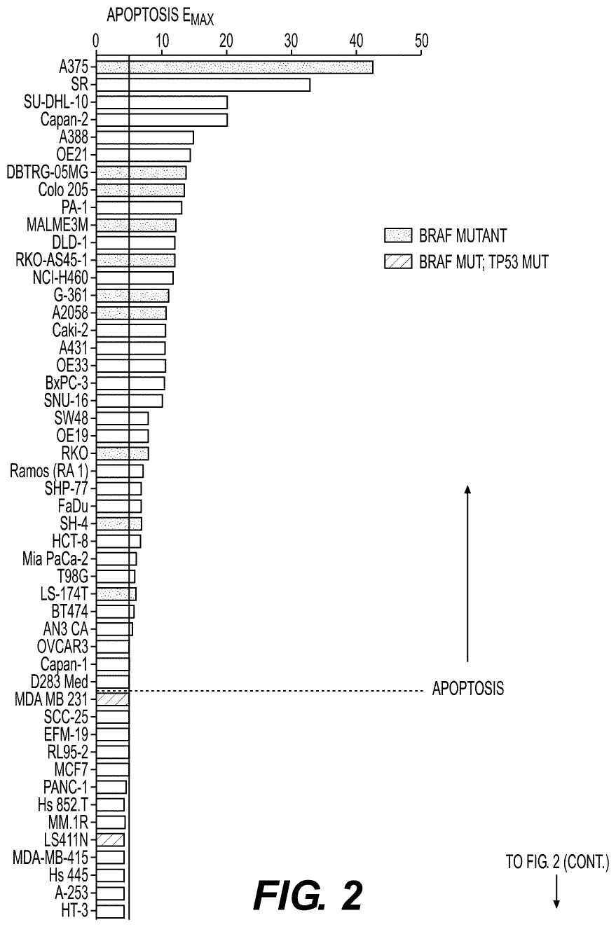

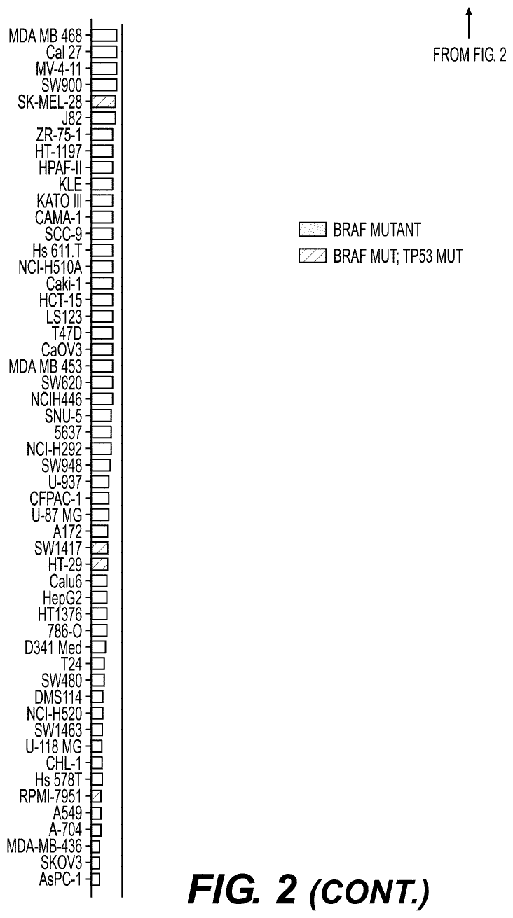

This enrichment of sensitivity in BRAF mutant cell lines was even more apparent when cell apoptosis was examined (FIG. 2). Of the 36 cell lines that showed significant induction of apoptosis (>5-fold induction of activated caspase-3), 10 were BRAF mutant, representing the majority of the BRAF mutant cell lines present in the cell line panel. Of the remaining cell lines (64) that did not show significant apoptosis, 6 were BRAF mutant as well as TP53 mutant.

Profiling of an eIF4E inhibitor across a panel of tumor cell lines in vitro revealed several patterns of proliferation and/or apoptosis sensitivity with respect to tumor type and mutational status. In particular, Hematological, head and neck, NSCLC, melanoma, uterine, and pancreatic tumor cell lines, were sensitive to eIF4E inhibition, consistent with the dysregulation of eIF4E in these tumor types. Importantly, the analysis also revealed that BRAF mutant cell lines were enriched in the population of cell lines showing heightened sensitivity to eIF4E inhibition, independent of tumor type.

### Example 2

In Vivo Tumor Growth Inhibition

To test whether eIF4E inhibition affected the growth of BRAF mutant tumors in vivo, COLO 205 cells were grown as tumor xenografts in athymic nude mice and animals were dosed daily with vehicle or Compound Y, having the following structure

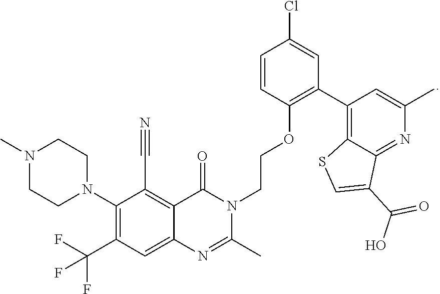

Athymic nude mice were purchased from Simonsen Laboratories (Gilroy, Calif.). C.B-17 SCID mice were purchased from Charles River (Hollister, Calif.). All animal studies were carried out in accordance with the guidelines established by the Institutional Animal Care and Use Committee at Explora BioLabs (San Diego, Calif.). COLO 205 and RKO cells were purchased from ATCC (Manassas, Va.). COLO 205 cells were maintained in RPMI-1640 supplemented with 10% fetal bovine serum. RKO cells were maintained in DMEM supplemented with 10% fetal bovine serum. For COLO 205 xenograft studies, COLO 205 (2×10⁶) cells were implanted subcutaneously into athymic nude mice. On day 8 post-implant, tumors were measured (˜103 mm³) and tumor-bearing animals were randomized into treatment groups. For RKO xenograft studies, RKO cells (5×10⁶) were implanted subcutaneously into C.B-17 SCID mice. On day 11 post-implant, tumors were measured (˜153 mm³) and tumor-bearing animals were randomized into treatment groups. Animals were dosed orally with vehicle or Compound Y daily. Tumor size and body weight were monitored during the course of the study. Tumor size was measured in length and width with a caliper 2× per week. The tumor volume was calculated by the formula L×W×W/2 according to NCI standards.

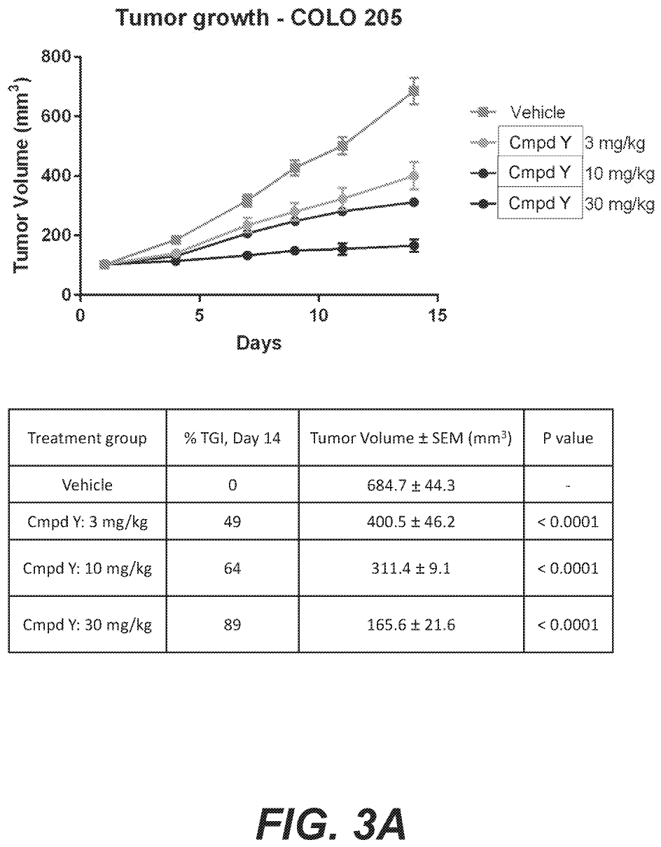

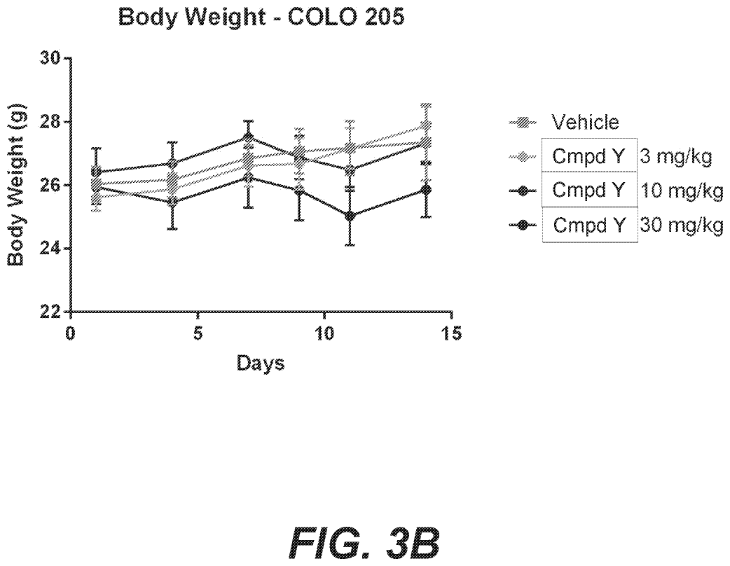

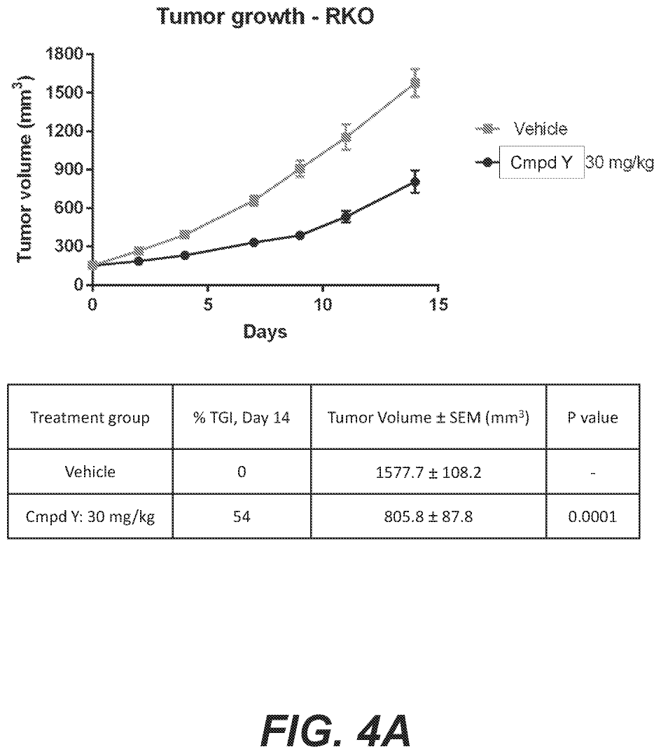

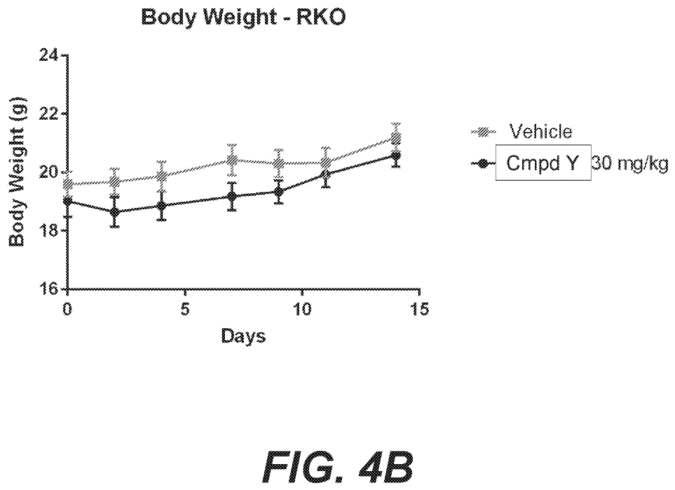

In animals treated with Compound Y, significant dose-dependent tumor growth inhibition (TGI) was observed, ranging from 49% at 3 mg/kg to 89% at 30 mg/kg (FIG. 3A). In RKO xenografts, another BRAF mutant tumor model, Compound Y treatment resulted in a TGI of 54% at 30 mg/kg (FIG. 4A). In both studies, Compound Y was well tolerated in the animals, as evidenced by no significant changes in body weight (FIGS. 3B and 4B). Taken together, these studies demonstrate that Compound Y has activity against BRAF mutant tumors in vivo at well-tolerated doses. These results demonstrate that BRAF mutational status could be used as a selection criterion for identifying a patient population that may clinically benefit from eIF4E inhibitors.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

SEQUENCES
[wild type human BRAF polypeptide]
SEQ ID NO: 1
MAALSGGGGGGAEPGQALFNGDMEPEAGAGAGAAASSAADPAIPEEVWN
IKQMIKLTQEHIEALLDKFGGEHNPPSIYLEAYEEYTSKLDALQQREQQ
LLESLGNGTDFSVSSSASMDTVTSSSSSSLSVLPSSLSVFQNPTDVARS
NPKSPQKPIVRVFLPNKQRTVVPARCGVTVRDSLKKALMMRGLIPECCA
VYRIQDGEKKPIGWDTDISWLTGEELHVEVLENVPLTTHNFVRKTFFTL
AFCDFCRKLLFQGFRCQTCGYKFHQRCSTEVPLMCVNYDQLDLLFVSKF
FEHHPIPQEEASLAETALTSGSSPSAPASDSIGPQILTSPSPSKSIPIP
QPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTL
GRRDSSDDWEIPDGQITVGQRIGSGSFGTVYKGKWHGDVAVKMLNVTAP
TPQQLQAFKNEVGVLRKTRHVNILLFMGYSTKPQLAIVTQWCEGSSLYH
HLHIIETKFEMIKLIDIARQTAQGMDYLHAKSIIHRDLKSNNIFLHEDL
TVKIGDFGLATVKSRWSGSHQFEQLSGSILWMAPEVIRMQDKNPYSFQS
DVYAFGIVLYELMTGQLPYSNINNRDQIIFMVGRGYLSPDLSKVRSNCP
KAMKRLMAECLKKKRDERPLFPQILASIELLARSLPKIHRSASEPSLNR
AGFQTEDFSLYACASPKTPIQAGGYGAFPVH
[human BRAF V600 substituted polypeptide, wherein
underlined V600 is substituted with any amino
acid other than V]
SEQ ID NO: 2
MAALSGGGGGGAEPGQALFNGDMEPEAGAGAGAAASSAADPAIPEEVWN
IKQMIKLTQEHIEALLDKFGGEHNPPSIYLEAYEEYTSKLDALQQREQQ
LLESLGNGTDFSVSSSASMDTVTSSSSSSLSVLPSSLSVFQNPTDVARS
NPKSPQKPIVRVFLPNKQRTVVPARCGVTVRDSLKKALMMRGLIPECCA
VYRIQDGEKKPIGWDTDISWLTGEELHVEVLENVPLTTHNFVRKTFFTL
AFCDFCRKLLFQGFRCQTCGYKFHQRCSTEVPLMCVNYDQLDLLFVSKF
FEHHPIPQEEASLAETALTSGSSPSAPASDSIGPQILTSPSPSKSIPIP
QPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTL
GRRDSSDDWEIPDGQITVGQRIGSGSFGTVYKGKWHGDVAVKMLNVTAP
TPQQLQAFKNEVGVLRKTRHVNILLFMGYSTKPQLAIVTQWCEGSSLYH
HLHIIETKFEMIKLIDIARQTAQGMDYLHAKSIIHRDLKSNNIFLHEDL
TVKIGDFGLATVKSRWSGSHQFEQLSGSILWMAPEVIRMQDKNPYSFQS
DVYAFGIVLYELMTGQLPYSNINNRDQIIFMVGRGYLSPDLSKVRSNCP
KAMKRLMAECLKKKRDERPLFPQILASIELLARSLPKIHRSASEPSLNR
AGFQTEDFSLYACASPKTPIQAGGYGAFPVH
[human BRAF K601 substituted polypeptide, wherein
underlined K601 is substituted with any amino
acid other than K]
SEQ ID NO: 3
MAALSGGGGGGAEPGQALFNGDMEPEAGAGAGAAASSAADPAIPEEVWN
IKQMIKLTQEHIEALLDKFGGEHNPPSIYLEAYEEYTSKLDALQQREQQ
LLESLGNGTDFSVSSSASMDTVTSSSSSSLSVLPSSLSVFQNPTDVARS
NPKSPQKPIVRVFLPNKQRTVVPARCGVTVRDSLKKALMMRGLIPECCA
VYRIQDGEKKPIGWDTDISWLTGEELHVEVLENVPLTTHNFVRKTFFTL
AFCDFCRKLLFQGFRCQTCGYKFHQRCSTEVPLMCVNYDQLDLLFVSKF
FEHHPIPQEEASLAETALTSGSSPSAPASDSIGPQILTSPSPSKSIPIP
QPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTL
GRRDSSDDWEIPDGQITVGQRIGSGSFGTVYKGKWHGDVAVKMLNVTAP
TPQQLQAFKNEVGVLRKTRHVNILLFMGYSTKPQLAIVTQWCEGSSLYH
HLHIIETKFEMIKLIDIARQTAQGMDYLHAKSIIHRDLKSNNIFLHEDL
TVKIGDFGLATVKSRWSGSHQFEQLSGSILWMAPEVIRMQDKNPYSFQS
DVYAFGIVLYELMTGQLPYSNINNRDQIIFMVGRGYLSPDLSKVRSNCP
KAMKRLMAECLKKKRDERPLFPQILASIELLARSLPKIHRSASEPSLNR
AGFQTEDFSLYACASPKTPIQAGGYGAFPVH
[BRAF L597 substituted polypeptide, wherein
underlined L597is substituted withany amino acid
other than L]
SEQ ID NO: 4
MAALSGGGGGGAEPGQALFNGDMEPEAGAGAGAAASSAADPAIPEEVWN
IKQMIKLTQEHIEALLDKFGGEHNPPSIYLEAYEEYTSKLDALQQREQQ
LLESLGNGTDFSVSSSASMDTVTSSSSSSLSVLPSSLSVFQNPTDVARS
NPKSPQKPIVRVFLPNKQRTVVPARCGVTVRDSLKKALMMRGLIPECCA
VYRIQDGEKKPIGWDTDISWLTGEELHVEVLENVPLTTHNFVRKTFFTL
AFCDFCRKLLFQGFRCQTCGYKFHQRCSTEVPLMCVNYDQLDLLFVSKF
FEHHPIPQEEASLAETALTSGSSPSAPASDSIGPQILTSPSPSKSIPIP
QPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTL
GRRDSSDDWEIPDGQITVGQRIGSGSFGTVYKGKWHGDVAVKMLNVTAP
TPQQLQAFKNEVGVLRKTRHVNILLFMGYSTKPQLAIVTQWCEGSSLYH
HLHIIETKFEMIKLIDIARQTAQGMDYLHAKSIIHRDLKSNNIFLHEDL
TVKIGDFGLATVKSRWSGSHQFEQLSGSILWMAPEVIRMQDKNPYSFQS
DVYAFGIVLYELMTGQLPYSNINNRDQIIFMVGRGYLSPDLSKVRSNCP
KAMKRLMAECLKKKRDERPLFPQILASIELLARSLPKIHRSASEPSLNR
AGFQTEDFSLYACASPKTPIQAGGYGAFPVH

What is claimed is:

1. A method of treating cancer, the method comprising administering to a subject having BRAF-mutated cancer cells an effective amount of an eIF4E inhibitor.

2. The method of claim 1, wherein the BRAF-mutated cancer cell comprises a mutation that activates BRAF.

3. The method of claim 2, wherein the mutation that activates BRAF comprises an amino acid substitution at V600, K601, L597, or any combination thereof.

4. The method of claim 3, wherein the V600 substitution is a V600E, a V600K, a V600D, a V600R, a V600M, or a V600G substitution.

5. The method of claim 3, wherein the K601 substitution is a K601E substitution.

6. The method of claim 3, wherein the L597 substitution is a L597Q, a L597R, a L597S, or a L597V substitution.

7. The method of any one of claims 1-6, wherein the BRAF-mutated cancer cell comprises one or more substitutions occurring at BRAF polypeptide positions selected from the group consisting of A29, H72, S113, S124, P162, C₁₉₄, L227, P231, C₂₅₁, V291, Q329, V483, L485, T521, V528, D587, P655, S657, S683, P686, C₆₉₆, L697, P722, F738, and C₇₄₈; wherein the BRAF polypeptide is a wild-type BRAF polypeptide (SEQ ID NO:1), a BRAF V600 polypeptide (SEQ ID NO:2), a BRAF K601 polypeptide (SEQ ID NO:3), or a BRAF L597 polypeptide (SEQ ID NO:4).

8. The method of claim 7, wherein the BRAF polypeptide having one or more amino acid substitutions are selected from the group consisting of A29V, H72N, S113I, S124F, P162H, C₁₉₄*, L227F, P231T, C₂₅₁F, V291F, Q329K, V483E, L485F, T521K, V528F, D587E, P655T, S657*, S683R, P686Q, P686T, C₆₉₆*, L697I, P722T, F738L, and C748F.

9. The method of claim 7 or 8, wherein the mutant BRAF polypeptide is a BRAF polypeptide comprising a substitution at one or more of amino acid positions T521, V528, and P686.

10. The method of claim 9, wherein the mutant BRAF polypeptide is a BRAF polypeptide comprising a substitution at one or more of amino acid positions T521K, V528F, and P686Q.

11. The method of any one of claims 1-10, wherein mutated BRAF is resistant to a RAF inhibitor.

12. The method of any one of claims 1-11, wherein the BRAF-mutated cancer cell is a melanoma, lung cancer, small cell lung cancer, non-small-cell lung cancer, head and neck squamous cell carcinoma, sarcoma, thyroid cancer, thyroid carcinoma, colon carcinoma, colorectal cancer, pancreatic cancer, gastric cancer, esophageal cancer, prostate cancer, breast cancer, ovarian cancer, laryngeal cancer, cervical cancer, lymphatic system cancer, genitourinary tract cancer, bone cancer, biliary tract cancer, endometrial cancer, liver cancer, brain cancer, glioblastoma, astrocytoma, ganglioglioma, craniopharyngioma, Langerhans cell histiocytosis, multiple myeloma, leukemia, hairy cell leukemia, or non-Hodgkin's lymphoma cell.

13. The method of any one of claims 1-12, wherein the subject does not have an activating K-ras mutation.

14. The method of claim 13, wherein the subject does not have a K-ras mutation of at least one of G12C, G12A, G12D, G12R, G12S, G12V, G13C, G13R, G13S, G13A, G13D, Q61K, Q61L, Q61R, and Q61H.

15. The method of any one of claims 1-14, further comprising administering to the subject an inhibitor of an immunosuppression component, a chemotherapeutic agent, or any combination thereof.

16. The method of claim 15, wherein the chemotherapeutic agent is a RAF inhibitor, MEK inhibitor, mTOR inhibitor, MNK specific inhibitor, eIF4A inhibitor, or any combination thereof.

17. The method of claim 16, wherein the RAF inhibitor is vemurafenib, dabrafenib, encorafenib, or RAF265.

18. The method of claim 16, wherein the MEK inhibitor is trametinib, cobimetinib, selumetinib, binimetinib, PD-325901, CI-1040, or PD035901.

19. The method of claim 16, wherein the mTOR inhibitor is rapamycin, sirolimus, temsirolimus, everolimus, ridaforolimus, zotarolimus, farnesylthiosalicylic acid, BEZ235, CCG168, PP242, INK128, Torin 1, Torin 2, or curcumin.

20. The method of any one of claims 1-19, wherein the eIF4E inhibitor is an eIF4G1 peptide, a modified eIF4G1 peptide, a cross-linked eIF4G1 peptide, or briciclib.

21. The method of any one of claims 1-19, wherein the eIF4E inhibitor is a compound according to Formula (I): 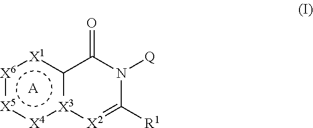
or stereoisomers, tautomers, or pharmaceutically acceptable salts thereof, wherein:
X¹ is CR², —C-L¹-Y or N;
X², X⁵ and X⁶ are independently CR² or N,
wherein X⁵ and X⁶ together with 3 or 4 carbon or nitrogen atoms combine to form a 5- or 6-membered cycloalkyl or heterocyclyl,
or when X² is CR², R¹ and R² together with the atoms they attached to form a 6-membered aryl or heteroaryl;
X³ is C, or X³ is C or N when X⁴ is a bond;
X⁴ is a bond, CR² or N,
wherein X⁴ and X⁵ together with 3 or 4 carbon or nitrogen atoms combine to form a 5- or 6-membered heteroaryl;
Q is H or -L¹-Y;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂CC— or —CH₂(cyclopropyl)—;
Y is 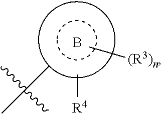
wherein
Ring B is a six-membered aryl, heteroaryl or heterocyclyl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O, ═S, SR⁷, SO₂R⁷, NH(CO)R⁷ or NR⁷(CO)R⁷;
R⁴ is H, OH, halo, CN, (C₁-C₃)alkyl, (C₁-C₃)haloalkyl, (C₁-C₃)alkoxy, SR⁷ or Z, wherein Z is 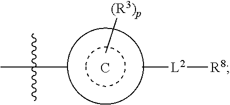
Ring C is cycloalkyl, heterocyclyl, aryl or heteroaryl;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
or wherein two R⁵ substituents together with a nitrogen atom form a 4-, 5-, 6- or 7-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈) alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl,
wherein when X⁴ is a bond ring A forms a 5-membered heteroaryl wherein X⁵ and X⁶ can in addition to the above defined substituents be NR², and X¹ can in addition be —N-L¹-Y; and
wherein either Q is -L¹-Y, or X¹ is —C-L¹-Y or —N-L¹-Y.

22. The method of any one of claims 1-19, wherein the eIF4E inhibitor is a compound according to Formula II: 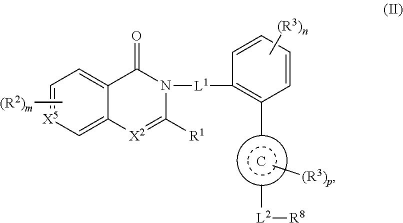
or stereoisomers, tautomers, or pharmaceutically acceptable salts thereof, wherein:
X² and X⁵ are independently CR² or N,
or when X² is CR², R¹ and R² together with the atoms they attached to form a 6-membered aryl or heteroaryl;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, OC(R⁶)(R⁶)—, —C(═O)—, —C(═O)N(R⁵)C(R⁶)(R⁶)— or a bond;
Ring C is cycloalkyl, heterocyclyl, aryl or heteroaryl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
or wherein two R⁵ substituents together with a nitrogen atom form a 4-, 5-, 6-, or 7-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
m is 0, 1, 2 or 3;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈) alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

23. The method of any one of claims 1-19, wherein the eIF4E inhibitor is a compound according to formula III: 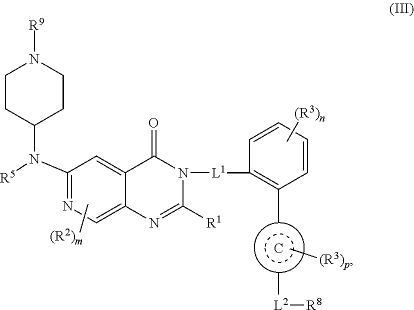
or stereoisomers, tautomers, or pharmaceutically acceptable salts thereof, wherein:
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
Ring C is a heteroaryl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁵ is independently H, (C₁-C₃)alkyl, (C₁-C₃)haloalkyl, (C₃-C₅)cycloalkyl or heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
R⁹ is H, (C₁-C₈)alkyl, (C₁-C₈)haloalkyl, cycloalkyl or heterocyclyl;
m is 0, 1, or 2;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl.

24. The method of claim 15, wherein the inhibitor of an immunosuppression component comprises an inhibitor of a PD-L1, PD-L2, CD80, CD86, B7-H3, B7-H4, HVEM, adenosine, GALS, VISTA, CEACAM-1, CEACAM-3, CEACAM-5, and PVRL2), PD-1, CTLA-4, BTLA, KIR, LAG3, TIM3, A2aR, CD244/2B4, CD160, TIGIT, LAIR-1, PVRIG/CD112R, IDO, arginase, TGFβ, IL-10, IL-35, or any combination thereof.

25. The method of claim 24, wherein the PD-1 inhibitor is pidilizumab, nivolumab, pembrolizumab, or any combination thereof.

26. The method of claim 24, wherein the PD-L1 inhibitor is avelumab, atezolizumab, durvalumab, MDX-1105 (BMS-936559), or any combination thereof.

27. The method of claim 24, wherein the CTLA4 inhibitor is tremelimumab, ipilimumab, or both.

28. The method of claim 16, wherein the MNK specific inhibitor is a compound according to the following formula: 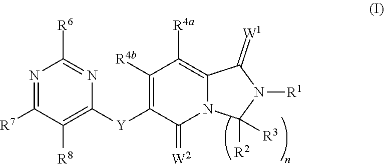
or is a stereoisomer, tautomer or pharmaceutically acceptable salt thereof, wherein:
W¹ and W² are independently O, S or N—OR′, where R′ is lower alkyl;
Y is —N(R⁵)—, —O—, —S—, —C(O)—, —S═O, —S(O)₂—, or —CHR⁹—;
R¹ is hydrogen, lower alkyl, cycloalkyl or heterocyclyl wherein any lower alkyl, cycloalkyl or heterocyclyl is optionally substituted with 1, 2 or 3 J groups;
n is 1, 2 or 3;
R² and R³ are each independently hydrogen, alkyl, alkenyl, alkynyl, aryl, araalkylene, heteroaryl, heteroarylalkylene, cycloalkyl, cycloalkylalkylene, heterocyclyl, or heterocyclylalkylene, wherein any alkyl, aryl, araalkylene, heteroaryl, heteroarylalkylene, cycloalkyl, cycloalkylalkylene, heterocyclyl, or heterocyclylalkylene, is optionally substituted with 1, 2 or 3 J groups;
or R² and R³ taken together with the carbon atom to which they are attached form a cycloalkyl or heterocyclyl, wherein any cycloalkyl or heterocyclyl is optionally substituted with 1, 2 or 3 J groups;
R^{4a} and R^{4b} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;
R⁵ is hydrogen, cyano, or lower alkyl;
or R⁵ and R⁸ taken together with the atoms to which they are attached form a fused heterocyclyl optionally substituted with 1, 2 or 3 J groups;
R⁶, R⁷ and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkylaminyl, heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;
or R⁷ and R⁸ taken together with the atoms to which they are attached form a fused heterocyclyl or heteroaryl optionally substituted with 1, 2 or 3 J groups;
J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)— cycloalkyl, —CHR⁹—C(O)-aryl, —CHR⁹-aryl, —C(O)-aryl, —CHR⁹—C(O)-heterocycloalkyl, —C(O)— heterocycloalkyl, heterocyclylaminyl, or heterocyclyl; or any two J groups bound to the same carbon or hetero atom may be taken together to form oxo; and
R⁹ is hydrogen, lower alkyl or —OH.

29. The method of claim 16, wherein the MNK-specific inhibitor is a compound according to the following formula: 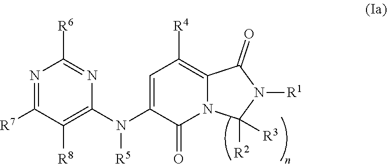
or a stereoisomer, tautomer or pharmaceutically acceptable salt thereof, wherein:
R¹ is hydrogen or lower alkyl;
n is 1, 2 or 3;
R² and R³ are independently and at each occurrence hydrogen, alkyl, carbocycle, carbocyclealkyl, heterocycle or heterocyclealkyl, wherein such alkyl, carbocycle, carbocyclealkyl, heterocycle or heterocyclealkyl is unsubstituted or substituted with 1, 2 or 3 J groups;
or R² and R³ taken together with the carbon atom to which they are attached form a carbocycle or heterocycle, wherein such carbocyclyl or heterocyclyl is unsubstituted or substituted with 1, 2 or 3 J groups;
R⁴ is hydrogen, halogen, alkyl, alkoxy, thioalkyl, alkenyl or cycloalkyl;
R⁵ is hydrogen or lower alkyl;
or R⁵ and R⁸ taken together with the atoms to which they are attached form a fused heterocycle unsubstituted or substituted with 1, 2 or 3 J groups;
R⁶, R⁷ and R⁸ are independently and at each occurrence hydrogen, halogen, alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl, each of which alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl is unsubstituted or substituted with 1, 2 or 3 J groups;
or R⁷ and R⁸ taken together with the atoms to which they are attached form a fused heterocycle unsubstituted or substituted with 1, 2 or 3 J groups; and
J is halogen, amino, alkyl, haloalkyl, cycloalkyl, amino or aminoalkyl, or when any two J groups are bound to the same carbon or hetero atom may be taken together to form oxo.

30. The method of claim 16, wherein the MNK-specific inhibitor is a compound according to the following formula: 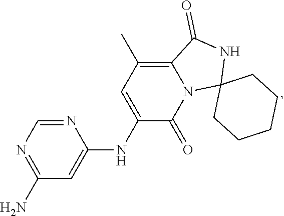 [SMILES: Cc1cc(Nc2cc(N)ncn2)c(=O)n2c1C(=O)NC21CCCCC1]
or is a stereoisomer, tautomer, or pharmaceutically acceptable salt thereof.

31. The method of claim 16, wherein the eIF4A inhibitor is inhibitor is a compound according to Formula (I): 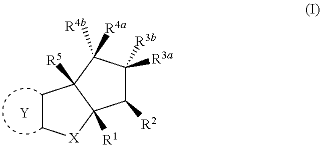
or is a stereoisomer, tautomer, or pharmaceutically acceptable salt thereof, wherein:
X is CR⁶R⁷, O, S, NH, N(C₁-C₈)alkyl, C(O), C═CR⁶R⁷, N(CO)R⁸, S(O) or S(O)₂;
Y is a 5-membered heteroaryl or a 6-membered aryl or heteroaryl;
R¹ and R² independently are aryl, heterocyclyl, heteroaryl or cycloalkyl;
R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹, NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹) (OR⁹), aryl, heteroaryl, cycloalkyl or heterocyclyl;
R^{3a} and R^{3b}, and R^{4a} and R^{4b} independently combine to form oxo or alkenyl, or a cycloalkyl or heterocyclyl ring; or
R^{3a} and R^{4a}, R^{3b} and R^{4b} or R^{4a} and R⁵ together with the carbon atom to which they are attached form a cycloalkyl or heterocyclyl ring; or
R² and R^{3a} together with the carbon atom to which they are attached form a bicyclic ring system;
R⁵ is H, halogen, OH, CN, N₃, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;
R⁶ and R⁷ independently are H, CN, halogen, OR⁹, SR⁹, (C₁-C₈)alkyl, NH(R⁹) or NR⁹R⁹;
R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);
R⁹ is H, (C₁-C₈)alkyl, (C₁-C₈)haloalkyl, cycloalkyl, heterocyclyl, [(C₁-C₈)alkylene]heterocyclyl, aryl, [(C₁-C₈)alkylene]aryl or heteroaryl;
wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;
wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂—[CH(OH)]ₘ—(CH₂)ₚ—OH, CH₂—[CH(OH)]ₘ-(CH₂)ₚ—NH₂ or CH₂-aryl-alkoxy;
or wherein any alkyl, cycloalkyl or heterocyclyl is optionally substituted with oxo;
“m” and “p” are 1, 2, 3, 4, 5 or 6; and
wherein when Y is a 6-membered aryl then X is not O.

32. The composition of claim 31, wherein the eIF4A inhibitor is a compound according to the following formula: 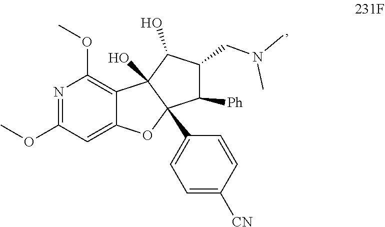
or is a stereoisomer, tautomer, or pharmaceutically acceptable salt thereof.

33. The method of any one of claims 15-32, wherein the inhibitor of an immunosuppression component or a chemotherapeutic agent is administered simultaneously, concurrently, or sequentially with the eIF4E inhibitor.

34. The method of any one of claims 1-33, wherein the subject is human.
